(19) United States
(12) Patent Application Publication
(10) Pub. No.: US 2025/0161501 A1
(43) Pub. Date: May 22, 2025

(54) Radiolabeled Compounds for Imaging of Fibroblast Activation Protein (FAP) and Treatment of FAP-Related Disorders

(71) Applicants: Provincial Health Services Authority, Vancouver (CA); The University of British Columbia, Vancouver (CA)

(72) Inventors: Kuo-Shyan Lin, Richmond (CA); François Bénard, Vancouver (CA); Zhengxing Zhang, Langley (CA); Shreya Bendre, Vancouver (CA)

(21) Appl. No.: 18/728,537

(22) PCT Filed: Jan. 13, 2023
(86) PCT No.: PCT/CA2023/050040

§ 371 Date: Jul. 12, 2024

Related U.S. Application Data
(60) Provisional application No. 63/299,429, filed on Jan. 14, 2022.

(51) Int. Cl. A61K 51/04; A61P 35/00; C07D 401/14; C07F 5/02

(52) U.S. Cl. CPC A61K 51/0497; A61P 35/00; C07D 401/14; C07F 5/025; A61K 2121/00

(57) ABSTRACT

Radiolabeled compounds that target fibroblast activation protein (FAP). The compounds have 1-6 radiolabeling groups and 1-6 FAP-targeting groups, connected by 1-11 linkers. FAP-targeting groups have the structure of Formula (I). R^{1a} and R^{1b} are each —H, —OH, halogen, C₁₋₆ alkyl, —O—C₁₋₆ alkyl, or —S—C₁₋₆ alkyl. R² is —NH—, —N(CH₃)—, —CH₂—, —CH(OH)—, —CHF—, —CF₂—, —S—, or —O—. R³ is —CO₂H, —C(O)NH₂, —CN or —B(OH)₂. R⁴ is —H, methyl, or ethyl. R⁵ is ═O. R⁶ is —C(O)—, —O—C(O)—, or —NH—C(O)—. n4 is 0, 1, 2, or 3. R⁷ is an 11 to 15-membered aromatic, partially aromatic, or non-aromatic fused tricyclic system, wherein the tricyclic system is optionally contains 1-6 heteroatoms selected from N, O, and/or S, and is optionally substituted. There is also provided the use of such compounds as imaging agents or therapeutic agents. Formula (I).

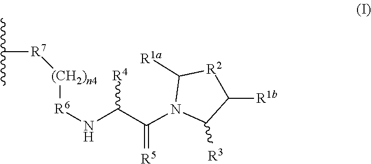

### INCORPORATION BY REFERENCE

This application claims priority to U.S. 63/299,429, which is incorporated by reference in its entirety.

### FIELD OF INVENTION

The present invention relates to radiolabelled compounds for in vivo imaging or treatment of diseases or conditions characterized by expression of the fibroblast activation protein.

### BACKGROUND OF THE INVENTION

The human fibroblast activation protein (FAP) is a transmembrane, non-classical serine protease belonging to the dipeptidyl peptidase (DPP) IV family which includes DPP IV, DPP6, DPP8 and DPP9 [1-2]. FAP is selectively expressed in reactive stromal fibroblasts or cancer associated fibroblasts (CAFs) of more than 90% epithelial carcinomas, malignant cells of some bone and soft tissue sarcomas, a subset of melanomas and most astrocytomas, but not in normal healthy tissues [3-6]. CAFs constitute a dominant component of the stroma and have been shown to promote and support tumor-cell survival through increased angiogenesis [7], T-cell mediated immunosuppression [8] and invasive and metastatic capabilities via extracellular matrix remodeling [9-10]. Due to its restricted expression pattern and the biological role of CAFs for promoting proliferation and invasion, FAP has long been recognized as a potential cancer imaging marker and therapeutic target [11-18].

An ¹³¹I-labeled mouse monoclonal antibody (mAb) F19 has been reported for detecting FAP-expressing hepatic metastases of colorectal cancer with SPECT [19]. However, the human anti-mouse IgG antibody response was observed in all cases 2-6 weeks after ¹³¹I-F19 administration. ¹⁷⁷Lu-labeled humanized mAbs (ESC11 and ESC14) were reported to successfully target FAP-expressing melanoma xenografts by SPECT imaging and shown to significantly delay tumor growth [20].

To overcome the slow pharmacokinetics of mAbs, the Haberkorn group designed and evaluated various ⁶⁸Ga-labeled FAP small-molecule inhibitors for PET imaging [21-24]. In clinical studies the top candidates, ⁶⁸Ga-FAPI-2 and ⁶⁸Ga-FAPI-4 (chemical structures shown below), successfully visualized 28 types of cancers in PET images [21-24]. However, the tumor uptake of ⁶⁸Ga-FAPI-2 and ⁶⁸Ga-FAPI-4 reduced quickly over time [25], which precludes radiotherapeutic application of their analogs labeled with cytotoxic radionuclides (α- and β-emitters). Therefore, novel FAP-targeting radioligands with higher tumor uptake and potentially longer tumor retention to improve detection sensitivity and treatment efficacy are urgently needed.

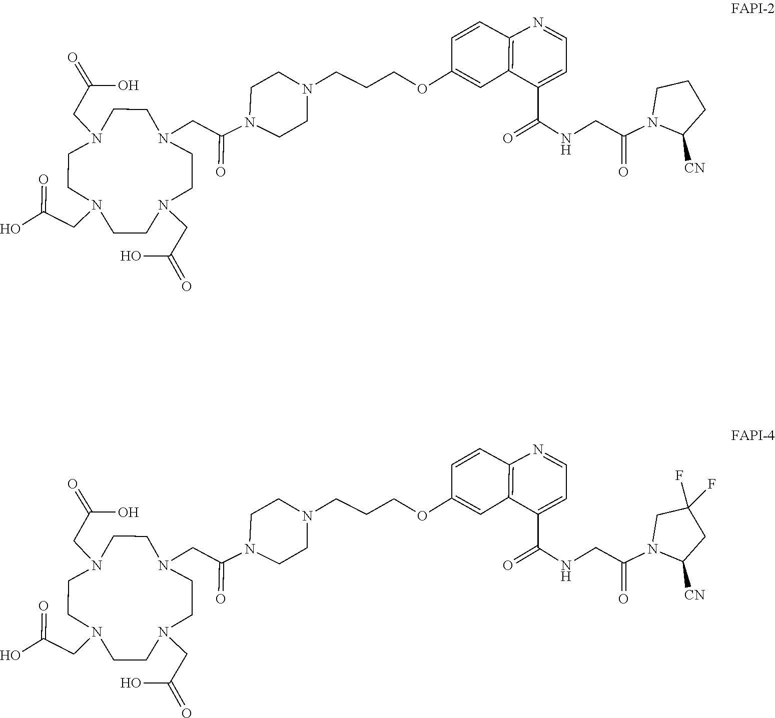 [SMILES: N#C[C@@H]1CCCN1C(=O)CNC(=O)c1ccnc2ccc(OCCCN3CCN(C(=O)CN4CCN(CC(=O)O)CCN(CC(=O)O)CCN(CC(=O)O)CC4)CC3)cc12 ; N#C[C@@H]1CC(F)(F)CN1C(=O)CNC(=O)c1ccnc2ccc(OCCCN3CCN(C(=O)CN4CCN(CC(=O)O)CCN(CC(=O)O)CCN(CC(=O)O)CC4)CC3)cc12]

There remains an unmet need in the field for improved tracers for the in-vivo imaging of the FAP, e.g. for detecting FAP-expressing diseases/disorders. There also remains an unmet need for improved radiotherapeutic agents for treatment of FAP-expressing diseases/disorders. In particular, there is a need for FAP-targeting agents that provide higher sensitivity for detection in imaging applications and increased efficacy in radiotherapeutic applications.

No admission is necessarily intended, nor should it be construed, that any of the preceding information constitutes prior art against the present invention.

### SUMMARY

This disclosure relates to radiolabelled fibroblast activation protein (FAP)-targeting compounds. Such compounds may be used for treatment or imaging of FAP-expressing diseases or conditions.

In one aspect, the compound comprises n1 radiolabeling groups and n2 FAP-targeting groups, wherein the radiolabeling groups and the FAP-targeting groups are connected through n3 linkers; n1 and n2 are each independently 1-6; n3 is 1-11; each of the FAP-targeting groups independently has the structure of Formula (I) or a pharmaceutically acceptable salt of Formula (I):

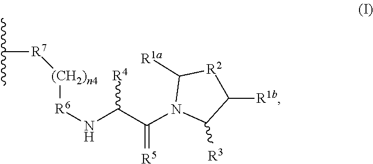

wherein: R^{1a} is —H, —OH, halogen, C₁₋₆ alkyl, —O—C₁₋₆ alkyl, or —S—C₁₋₆ alkyl; R^{1b} is —H, —OH, halogen, C₁₋₆ alkyl, —O—C₁₋₆ alkyl, or —S—C₁₋₆ alkyl; R² is —NH—, —N(CH₃)—, —CH₂—, —CH(OH)—, —CHF—, —CF₂—, —S—, or —O—; R³ is —CN or —B(OH)₂; R⁴ is —H, methyl, or ethyl; R⁶ is ═O; R⁶ is —C(O)—, —O—C(O)—, or —NH—C(O)—; n4 is 0, 1, 2, or 3; and R⁷ is an 11 to 15-membered aromatic, partially aromatic, or non-aromatic fused tricyclic system, wherein the tricyclic system is optionally contains 1-6 heteroatoms selected from N, O, and/or S, and is optionally substituted with 1-5 —OH, —NO₂, —CN, —NH₂, —NH(CH₃), —N(CH₃)₂, halogen, C₁₋₆ alkyl, —O—C₁₋₆ alkyl, or —S—C₁₋₆ alkyl; each of the radiolabeling groups is represented by R^{rad} wherein each R^{rad} is independently: a radiometal chelator; an aryl or heteroaryl substituted with a radiohalogen; a prosthetic group containing a trihaloborate; a prosthetic group containing a silicon-halogen-acceptor moiety; or a prosthetic group containing a halophosphate, halosulfate, or sulfonyl halide; each of the linkers is represented by R⁸, optionally wherein each R³ is a linear or branched chain of n5 units of R^{L1}, each unit of R^{L1} separated from each other by a unit of L¹, and each unit of R^{L1} optionally connected to an additional unit of L¹ to form a branching point, wherein each FAP-targeting group is connected to R³ through a unit of L³, and each R^{rad} is connected to R⁸ through a unit of L¹ or incorporates L¹ or a portion of L¹, wherein: n5 is 1-20; each R^{L1} is, independently, a linear, branched, and/or cyclic Cₙ₆ alkylenyl, alkenylenyl and/or alkynylenyl, wherein each n6 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid; L¹ bonds to carbon, wherein each L¹ is independently —O—, —S—, —N(R^{L2})—, —N(R^{L2})C(O)—, —N(R^{L2})—(S), —C(O)N(R^{L2}), —C(S)N(R^{L2})—, —NH—C(O)—NH—, —NH—C(S)—NH—,

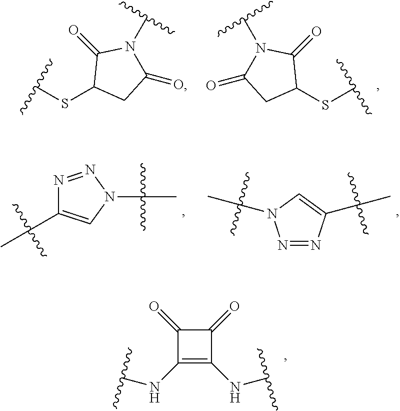

or L²; each R^{L2} is independently H, methyl, or ethyl; each L² is an N-containing heterocycle independently selected from the group consisting of:

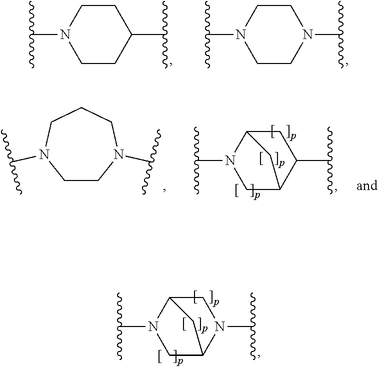

wherein each p is independently 1 or 2; and an albumin binder (R^{alb}) is optionally bonded to an L¹ of the linker, wherein each albumin binder is independently: —(CH₂)ₙ₇—CH₃ wherein n7 is 8-20; —(CH₂)ₙ₈—C(O)OH wherein n8 is 8-20;

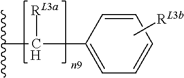

wherein n9 is 1-4 and R^{L3a} is H or methyl, and R^{L3b} is I, Br, F, Cl, H, OH, OCH₃, NH₂, NO₂ or C₁-C₆ alkyl; or

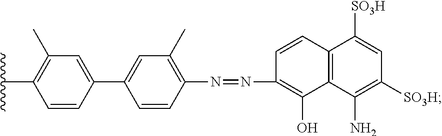 [SMILES: *c1ccc(-c2ccc(N=Nc3ccc4c(S(=O)(=O)O)cc(S(=O)(=O)O)c(N)c4c3O)c(C)c2)cc1C]

each L³ is independently: absent, —O—, —S—, —N(R^{L2})—, —N(R^{L2})C(O)—, —N(R^{L2})C(S)—, —C(O)N(R^{L2})—, —C(S)N(R^{L2})—, —NH—C(O)—NH—, —NH—C(S)—NH—,

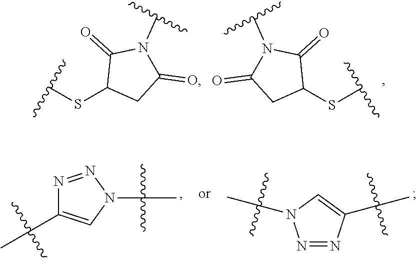

or L³ is absent, —C(O)—, —C(S)—, —N(R^{L2})C(O)—, —N(R^{L2})C(S)—, if bonded to nitrogen of the tricyclic system.

In another aspect, the compound has the structure of Formula (II) or is a pharmaceutically acceptable salt of Formula (II):

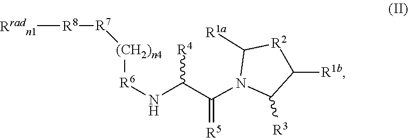

wherein n1, n4, R^{1a}, R^{1b}, R², R³, R⁴, R⁵, R⁶, R⁷, R⁸, and R^{rad} are as defined above.

In various embodiments, each FAP-targeting group has the structure of Formula (Ia) or a pharmaceutically acceptable salt of Formula (Ia):

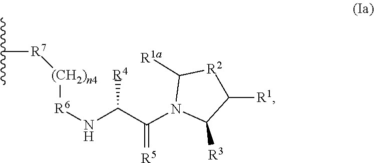

wherein n4, R^{1a}, R^{1b}, R², R³, R⁴, R⁵, R⁶, and R⁷ are as defined above.

In various embodiments, at least one R⁷ is:

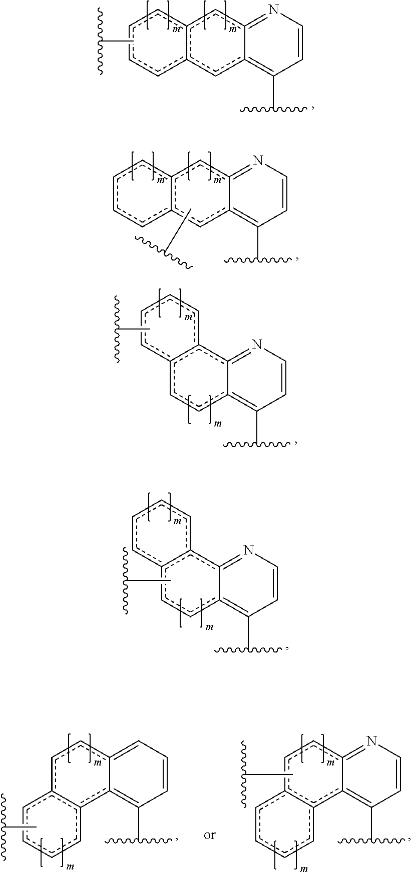

wherein the dashed bond indicates a single or double bond, wherein each m is independently 0 or 1, and the tricyclic system optionally contains 1-5 additional heteroatoms selected from N, O, and/or S, and is optionally substituted with 1-5 —OH, —NO₂, —CN, —NH₂, —NH(CH₃), NH(CH₃)₂, halogen, C₁₋₆ alkyl, —O—C₁₋₆ alkyl, or —S—C₁₋₆ alkyl. In certain embodiments, at least one R⁷ is:

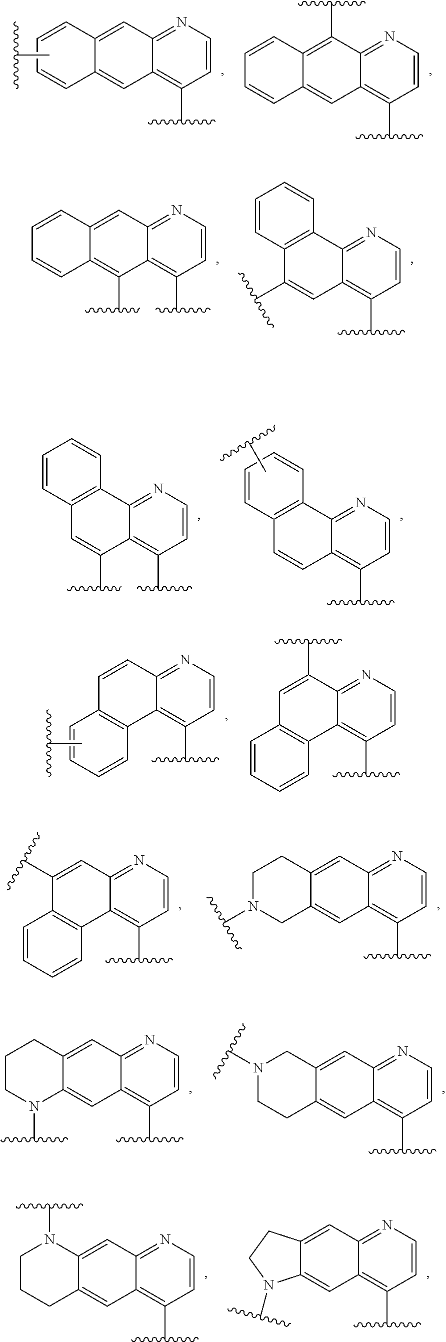

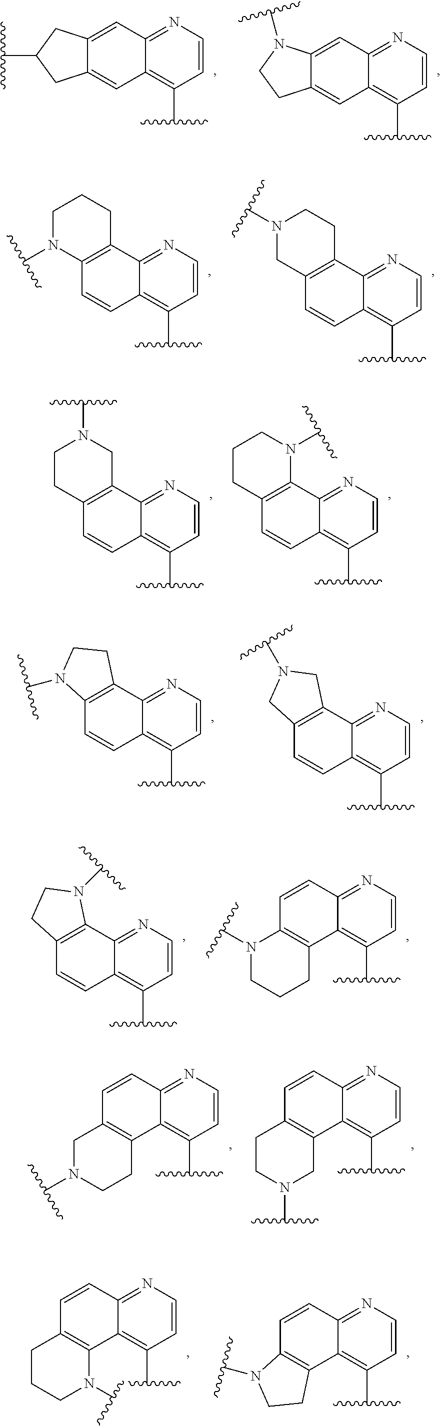

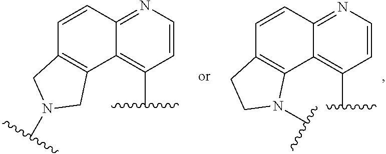 [SMILES: *c1ccnc2ccc3c(c12)CN(*)C3 ; *c1ccnc2ccc3c(c12)N(*)CC3]

wherein the tricyclic system optionally contains 1-3 additional heteroatoms selected from N, O, and/or S, and is optionally substituted with 1-5 —OH, —NO₂, —CN, —NH₂, —NH(CH₃), NH(CH₃)₂, halogen, C₁₋₆ alkyl, —O—C₁₋₆ alkyl, or —S—C₁₋₆ alkyl. In certain embodiments, at least one R⁷ is:

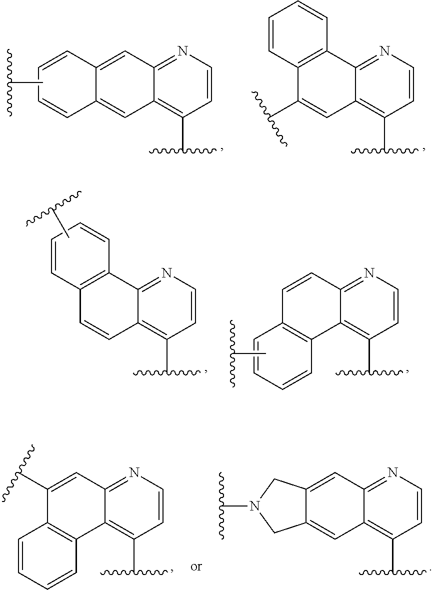

In another aspect, the compound is SB03178 or SB04033 or is a pharmaceutically acceptable salt thereof.

In some embodiments, the radiolabeling group(s) comprise a radionucleotide.

### BRIEF DESCRIPTION OF THE DRAWINGS

The features of the invention will become apparent from the following description in which reference is made to the appended drawings wherein:

FIG. 1 shows representative maximum-intensity-projection PET images of ⁶⁸Ga-FAPI-4 and ⁶⁸Ga-SB03178 acquired at 1 and 3 h post-injection from HEK293T:hFAP tumor-bearing mice.

FIG. 2 shows representative maximum-intensity-projection PET images of ⁶⁸Ga-SB03178 acquired at 1 and 3 h post-injection from U87 tumor-bearing mice.

FIG. 3 shows a representative maximum-intensity-projection PET image of ⁶⁸Ga-SB04033 acquired at 1 post-injection from HEK293T:hFAP tumor-bearing mice.

### DETAILED DESCRIPTION

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a compound, composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

The term “for example” or “e.g.” will be understood be refer to a non-limiting example(s) synonymous with the expression “for example, but without limitation”.

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, et cetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radionuclide” includes radionuclides that are suitable for use in imaging agents.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, solvates, salts or pharmaceutically acceptable salts thereof. Unless otherwise specified or indicated, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g. protonated) state or may be shown without specifying formal charges. As will be appreciated by the person of skill in the art, the ionization state of certain groups within a compound (e.g. without limitation, COOH, and the like) is dependent, inter alia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. As used herein, “pharmaceutically acceptable” means suitable for in vivo use in a subject, and is not necessarily restricted to therapeutic use, but also includes diagnostic use. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety.

As used herein, the expression “Cₙ” where n is an integer (e.g. 1, 2, 3, 4, 5, 6, 7, 8, 9, 10, 11, 12, 13, 14, 15, 16, 17, 18, 19, 20, and the like) or where n is defined as a range of integers (e.g. 1-20, 1-18, 2-15, 3-20, and the like) refers to the number of carbons in a compound, R-group, L-group, or substituent, or refers to the number of carbons plus heteroatoms in a compound, R-group, L-group, or substituent. A range of integers includes all integers in the range; e.g. the range 1-20 includes the integers 1, 2, 3, 4, 5, 6, 7, 8, 9, 10, 11, 12, 13, 14, 15, 16, 17, 18, 19, and 20. Unless otherwise defined, heteroatoms may include any, some or all possible heteroatoms. For example, in some embodiments, the heteroatoms may be selected from N, O, S, P and Se. In some embodiments, the heteroatoms are selected from N, S, or O. Such embodiments are non-limiting. The alternative expression “Cy-Cz”, where y and z are integers (e.g. C₃-C₁₅ and the like), is equivalent to “Cₙ” where n is a range of integers from y to z.

The terms “alkyl”, “alkylenyl”, “alkenylenyl”, and “alkynylenyl” have their usual meanings in organic chemistry. For example, an “alkyenylenyl” has at least one carbon-carbon double bond, and may have any number of carbon-carbon single bonds. Similarly, an “alkynylenyl” has at least one carbon-carbon triple bond, and may have any number of carbon-carbon single bonds. The expressions “alkylenyl, alkenylenyl and/or alkynylenyl” and “alkylenyl, alkenylenyl or alkynylenyl” are intended to be equivalent and each includes hydrocarbon chains that can have any reasonable number or combination of carbon-carbon single bonds, double bonds, and triple bonds. These hydrocarbon chains can be linear, branched, cyclic, or any combination of linear and branched, linear and cyclic, cyclic and branched, branched and cyclic, or linear, branched and cyclic. Cyclic hydrocarbons may be nonaromatic, partially aromatic, or aromatic. Unless otherwise specified, the term “cyclic” includes single rings, multiple non-fused rings, fused rings, bridged rings, and combinations thereof.

The expression “wherein any carbon . . . is optionally independently replaced by N, S, or O” and other similar expressions means that the defined hydrocarbon (e.g. “alkyl”, “alkylenyl”, “alkenylenyl”, or “alkynylenyl”) includes zero, one, more than one, or any reasonable combination of two or more heteroatoms selected from N, S, and O. The above expression therefore expands the defined hydrocarbon to additionally encompass heteroalkyls, heteroalkylenyls, heteroalkenylenyls, and heteroalkynylenyls, etc. The person of skill in the art would understand that various combinations of different heteroatoms may be used. The expression “wherein the tricyclic system optionally contains . . . additional heteroatoms selected from N, O, and/or S” and similar terms refer to replacing one or more carbons with one or more heteroatoms, including N heteroatom(s), O heteroatom(s), S heteroatom(s), or a combination of N and O heteroatoms, a combination of O and S heteroatoms, a combination N and S heteroatoms, or a combination of N, O, and S heteroatoms. The expression “wherein any carbon bonded to two other carbons is optionally independently replaced by N, S, or O” and other similar expressions means that any carbon in the defined hydrocarbon bonded to two other carbons (e.g. the underlined carbon in —C—C—C—), whether those bonds are single, double, or triple bonds, may be a heteroatom, but excludes heteroatoms bonded to other heteroatoms (e.g. excludes —C—N—S—, —S—S—N—, —N—S—C—, and the like).

Various R-groups (e.g. R¹, R², R³, etc.) and L-groups (e.g. L¹, L², etc.) are defined in this disclosure. L-groups generally refer to linkages (e.g. —O—, —S—, —NH—, —N(alkyl)-, —NH—C(O)—, —C(O)—NH—, —N(alkyl)-C(O)—, —C(O)—N(alkyl)-, —NH—C(O)—NH—, —NH—C(S)—NH—,

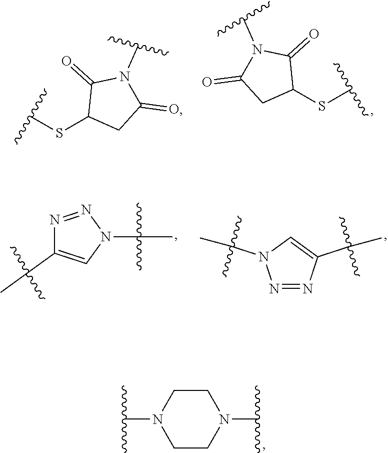

and the like).

If unspecified, the size of an R-group or L-group is what would be considered reasonable to the person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons in length, subject to the common general knowledge of the person of skill in the art. Further, but without limitation, if unspecified, the size of a heteroalkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons and heteroatoms in length, subject to the common general knowledge of the person of skill in the art. In the context of the expression “alkyl, alkenyl or alkynyl” and similar expressions, the “alkyl” would be understood to be a saturated alkyl, and the “alkenyl” and the “alkynyl” would be understood to be unsaturated.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

The term “alkylenyl” refers to a divalent analog of an alkyl group. In the context of the expression “alkylenyl, alkenylenyl and/or alkynylenyl”, and similar expressions, the “alkylenyl” would be understood to be a saturated alkylenyl, and the “alkenylenyl” and the “alkynylenyl” would be understood to be unsaturated. The term “heteroalkylenyl” refers to a divalent analog of a heteroalkyl group. The term “heteroalkenylenyl” refers to a divalent analog of a heteroalkenyl group. The term “heteroalkynylenyl” refers to a divalent analog of a heteroalkynyl group.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, l-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, l-propene-2-yl, 1-butene-l-yl, l-butene-2-yl, l-butene-3-yl, 2-butene-l-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. Unless otherwise specified, a C₁-C₂₀ alkynylenyl therefore encompasses, without limitation, all divalent analogs of the above-listed alkynyl groups.

Non-limiting examples of non-aromatic cyclic groups include cylcopropyl, cyclobutyl, cyclopentyl, cyclohexyl, and the like. Non-limiting examples of non-aromatic heterocyclic groups include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. non-limiting examples of C₃-C₂₀ aryl groups include phenyl (Ph), pentalenyl, indenyl, naphthyl and azulenyl. Non-limiting examples of aromatic heterocyclic groups of similar size include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like.

As used herein, the term “substituted” is used as it would normally be understood to a person of skill in the art and generally refers to a compound or chemical entity that has one chemical group replaced with a different chemical group. Unless otherwise specified, a substituted alkyl, alkylenyl, alkenylenyl, or alkynylenyl has one or more hydrogen atom(s) independently replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g. R-group or L-group) may be substituted with any chemical group reasonable to the person of skill in the art. For example, but without limitation, a hydrogen bonded to a carbon or heteroatom (e.g. N) may be substituted with halide (e.g. F, I, Br, CI), amine, amide, oxo, hydroxyl, thiol, phosphate, phosphonate, sulfate, SO₂H, SO₃H, alkyls, heteroalkyls, aryl, heteroaryl, ketones, carboxaldehyde, carboxylates, carboxamides, nitriles, monohalomethyl, dihalomethyl or trihalomethyl. In some embodiments, each carbon may be independently substituted or unsubstituted with oxo, hydroxyl, sulfhydryl, amine, amide, urea, halogen, guanidino, carboxylic acid, sulfonic acid, sulfinic acid, phosphoric acid, —NO₂, —CN, —NH(CH₃), —NH(CH₃)₂, C₁₋₆ alkyl, —O—C₁₋₆ alkyl, or —S—C₁₋₆ alkyl. In some embodiments, the amide substituent is —C(O)—NH₂.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”. The expression “optionally independently substituted” means that each location may be substituted or may not be substituted, and when substituted each substituent may be the same or different.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or alkyl (e.g. methyl) or R^{a} and R^{b} may form a cyclic structure. The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g. Asp, Glu, etc.) may be bonded to and the amine of another amino acid residue in the peptide (e.g. Dap, Dab, Orn, Lys). Further details are provided below. Unless otherwise indicated, “Xaa” may be any amino acid, including a proteinogenic or nonproteinogenic amino acid. Non-limiting examples of nonproteinogenic amino acids are shown in Table 1 and include: D-amino acids (including without limitation any D-form of the following amino acids), ornithine (Orn), 3-(1-naphtyl)alanine (Nal), 3-(2-naphtyl)alanine (2-Nal), α-aminobutyric acid, norvaline, norleucine (Nle), homonorleucine, beta-(1,2,3-triazol-4-yl)-L-alanine, 1,2,4-triazole-3-alanine, Phe(4-F), Phe(4-Cl), Phe(4-Br), Phe(4-I), Phe(4-NH₂), Phe(4-NO₂), homoarginine (hArg), 2-amino-4-guanidinobutyric acid (Agb), 2-amino-3-guanidinopropionic acid (Agp), B-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl) propanoic acid, 2-amino-3-(pyren-1-yl)propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), tranexamic acid, tert-leucine (Tle), 4-chlorophenylalanine (Cpa), thiazoline-4-carboxylic acid (Thz), aMe-Trp, p-aminomethylaniline-diglycolic acid (pABzA-DIG), 4-amino-1-carboxymethyl-piperidine (Pip), NH₂(CH₂)₂O(CH₂)₂C(O)OH, NH₂(CH₂)₂[O(CH₂)₂]₂C(O)OH (dPEG2), NH₂(CH₂)₂[O(CH₂)₂]₃C(O)OH, NH₂(CH₂)₂[O(CH₂)₂]₄C(O)OH, NH₂(CH₂)₂[O(CH₂)₂]₅C(O)OH, NH₂(CH₂)₂[O(CH₂)₂]₆C(O)OH, oxazolidine-4-carboxylic acid (4-oxa-L-Pro), β-(3-benzothienyl)alanine (Bta), citrulline (Cit), Trp(Me), Trp (7-Me), Trp(6-Me), Trp(5-Me), Trp(4-Me), Trp(2-Me), Trp(7-F), Trp(5-F), Trp(4-F) or cyclopentylglycine (Cpa). If not specified as an L- or D-amino acid, an amino acid shall be understood to be an L-amino acid.

TABLE 1
List of non-limiting examples of non-proteinogenic amino acids.

| | |
|---|---|
| p-aminomethylaniline-diglycolic acid (pABzA-DIG) | 2-amino-3-(anthracen-2-yl)propanoic acid |
| ornithine (Orn) | 2-amino-3-(anthracen-9-yl)propanoic acid |
| 3-(1-naphtyl)alanine (Nal) | 2-amino-3-(pyren-1-yl)propanoic acid |
| 3-(2-naphtyl)alanine (2-Nal) | Trp(5-Br), |
| α-aminobutyric acid | Trp(5-OCH₃), |
| norvaline | Trp(6-F) |
| norleucine (Nle) | Trp(5-OH) |
| homonorleucine | Trp(CHO) |
| beta-(1,2,3-triazol-4-yl)-L-alanine | N^{ε},N^{ε},N^{ε}-trimethyl-lysine |
| 1,2,4-triazole-3-alanine | cysteic acid |
| Phe(4-F), Phe(2-F), Phe(3-F), | 2-aminoadipic acid (2-Aad) |
| Phe(4-Cl), Phe(2-Cl), Phe(3-Cl), | 3-aminoadipic acid (3-Aad) |
| Phe(4-Br), Phe(2-Br), Phe(3-Br), | propargylglycine (Pra) |
| Phe(4-I), Phe(2-I), Phe(2-I), | homopropargylglycine (Hpg) |
| Phe(4-NH₂), Phe(2-NH₂), Phe(3-NH₂), | beta-homopropargylglycine (Bpg) |
| Phe(4-NO₂), Phe(2-NO₂), Phe(2-NO₂), | 2,3-diaminopropionic acid (Dap) |
| homoarginine (hArg) | 2,4-diaminobutyric acid (Dab) |
| 4-(2-aminoethyl)-1-carboxymethyl-piperazine (Acp) | azidolysine (Lys(N₃)) |
| 2-(5′-azidopentyl)alanine, 2-(6′-azidohexyl)alanine | azido-ornithine (Orn(N₃)) |
| 2-amino-4-guanidinobutyric acid (Agb) | amino-4-azidobutanoic acid Dab(N₃) |
| 2-amino-3-guanidinopropionic acid (Agp) | tranexamic acid |
| β-alanine | 4-amino-1-carboxymethyl-piperidine (Pip) |
| 4-aminobutyric acid | NH₂(CH₂)₂O(CH₂)₂C(O)OH |
| 5-aminovaleric acid | NH₂(CH₂)₂[O(CH₂)₂]₂C(O)OH (dPEG2) |
| 6-aminohexanoic acid | NH₂(CH₂)₂[O(CH₂)₂]₃C(O)OH |
| 7-aminoheptanoic acid | NH₂(CH₂)₂[O(CH₂)₂]₄C(O)OH |
| 8-aminooctanoic acid | NH₂(CH₂)₂[O(CH₂)₂]₅C(O)OH |
| 9-aminononanoic acid | NH₂(CH₂)₂[O(CH₂)₂]₆C(O)OH |
| 10-aminodecanoic acid | citrulline (Cit) |
| 2-aminooctanoic acid | β-(3-benzothienyl)alanine (Bta) |
| tert-leucine (Tle) | oxazolidine-4-carboxylic acid (4-oxa-L-Pro) |
| 4-chlorophenylalanine (Cpa) | cyclopentylglycine (Cpg) |
| thiazoline-4-carboxylic acid (Thz) | 5,5-dimethyl-1,3-thiazolidine-4-carboxylic |
| | acid (Me₂Thz) |
| αMe-Trp | any D-amino acid of a proteinogenic amino |
| | acid or any D-amino acid of a |
| | non-proteinogrenic amino acid in this Table |
| Trp(Me) | Trp(7-Me) |
| Trp(6-Me) | Trp(5-Me) |
| Trp(4-Me) | Trp(2-Me) |
| Trp(7-F) | Trp(5-F) |
| Trp(4-F) | any N-methylated version of a proteinogenic |
| | amino acid or non-proteinogenic amino acid |
| | in this Table |

The wavy line “ [symbol] ” symbol shown through or at the end of a bond in a chemical formula (e.g. left of R⁷ in Formula I) is intended to define the group on one side of the wavy line, without modifying the definition of the structure on the opposite side of the wavy line. Where an R-group or L-group is bonded on two or more sides, any atoms shown outside the wavy lines are intended to clarify orientation of the defined group. As such, only the atoms between the two wavy lines constitute the definition of the R-group or L-group. When atoms are not shown outside the wavy lines (e.g. L¹), or for a chemical group shown without wavy lines but does have bonds on multiple sides (e.g. —C(O)NH—, and the like), the chemical group should be read from left to right matching the orientation in the formula that the group relates to; e.g. for formula —R^{a}—R^{b}—R^{c}—, the definition of R^{b} as —C(O)NH— would be incorporated into the formula as —R^{a}—C(O)NH—R^{c}— not as —R^{a}—NHC(O)—R^{c}—. A wavy single bond (e.g. see R³ and R⁴ in Formula I) indicates that stereochemistry at a chiral center is unspecified and encompasses multiple isomers).

In various aspects, there is disclosed a compound comprising n1 radiolabeling groups (each represented by R^{rad}) and n2 fibroblast activation protein (FAP)-targeting groups (each represented by R^{FAP}), wherein the radiolabeling groups and the FAP-targeting groups are connected through n3 linkers (each represented by R⁸). n1 and n2 are each independently 1-6 (1, 2, 3, 4, 5, or 6). n3 is 1-11 (1, 2, 3, 4, 5, 6, 7, 8, 9, 10, or 11). Each radiolabeling group is the same or different (or a combination where some radiolabeling groups are the same and some are different). Each FAP-targeting group is the same or different (or a combination where some FAP-targeting groups are the same and some are different).

In some embodiments, n1 is 1. In some embodiments, n1 is 2. In some embodiments, n1 is 3.

In some embodiments, n1 is 4. In some embodiments, n1 is 5. In some embodiments, n1 is 6.

In some embodiments, n2 is 1. In some embodiments, n2 is 2. In some embodiments, n2 is 3.

In some embodiments, n2 is 4. In some embodiments, n2 is 5. In some embodiments, n2 is 6.

In some embodiments, n3 is 1. In some embodiments, n3 is 2. In some embodiments, n3 is 3.

In some embodiments, n3 is 4. In some embodiments, n3 is 5. In some embodiments, n3 is 6. In some embodiments, n3 is 7. In some embodiments, n3 is 8. In some embodiments, n3 is 9. In some embodiments, n3 is 10. In some embodiments, n3 is 11.

In some embodiments, each of n1 and n2 is 1, optionally wherein n3 is 1. In some embodiments, n1 is 2 and n2 is 2, wherein each radiolabeling group is the same or different, and each FAP-targeting group is the same or different, optionally wherein n3 is 1. In some embodiments, n1 is 1 (e.g. hexameric isocyanide coordinated to a metal ion), n2 is 6, and n3 is 6.

Each of the FAP-targeting groups (i.e. each R^{FAP}) independently has the structure of Formula (I) or a pharmaceutically acceptable salt of Formula (I):

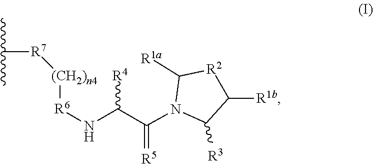

wherein:
R^{1a} is —H, —OH, halogen, C₁₋₆ alkyl, —O—C₁₋₆ alkyl, or —S—C₁₋₆ alkyl;
R^{1b} is —H, —OH, halogen, C₁₋₆ alkyl, —O—C₁₋₆ alkyl, or —S—C₁₋₆ alkyl;
R² is —NH—, —N(CH₃)—, —CH₂—, —CH(OH)—, —CHF—, —CF₂—, —S—, or —O—;
R³ is —H, —CN, —B(OH)₂, —CO₂H, —CONH₂, —SO₃H, —SO₂NH₂, —PO₃H₂, or 5-tetrazolyl;
R⁴ is —H, methyl, or ethyl;
R⁵ is ═O or ═S;
R⁶ is —C(O)—, —C(S)—, —O—C(O)—, —NH—C(O)—, or —NH—C(S)—;
n4 is 0, 1, 2, or 3; and
R⁷ is an 11 to 15-membered aromatic, partially aromatic, or non-aromatic fused tricyclic system, wherein the tricyclic system is optionally contains 1-6 heteroatoms selected from N, O, and/or S, and is optionally substituted with 1-5 —OH, —NO₂, —CN, —NH₂, —NH(CH₃), —N(CH₃)₂, halogen, C₁₋₆ alkyl, —O—C₁₋₆ alkyl, or —S—C₁₋₆ alkyl.

In some embodiments, the compound has the structure of Formula (II) or is a pharmaceutically acceptable salt of Formula (II), wherein n1, n4, R^{1a}, R^{1b}, R², R³, R⁴, R⁵, R⁶, and R⁷ define a FAP-targeting group and are as defined for Formula (I). R³ represents a linker. R^{rad} represents a radiolabeling group.

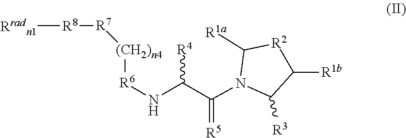

In some embodiments, each FAP-targeting group (i.e. R^{FAP}) has the structure of Formula (Ia) or a pharmaceutically acceptable salt of Formula (Ia), wherein n4, R^{1a}, R^{1b}, R², R³, R⁴, R⁵, R⁶, and R⁷ are as defined for Formula (I).

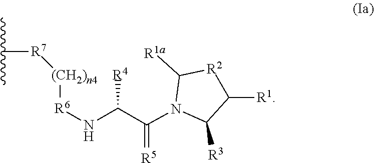

In some embodiments, at least one R^{1a} is —H. In some embodiments, at least one R^{1a} is —OH. In some embodiments, at least one R^{1a} is halogen; optionally the halogen is F, Cl, Br, or I. In some embodiments, at least one R^{1a} is C₁₋₆ alkyl, optionally methyl, ethyl, isopropyl, sec-butyl, isobutyl, or tert-butyl. In some embodiments, at least one R^{1a} is —O—C₁₋₆ alkyl, optionally —O-methyl or —O-ethyl. In some embodiments, at least one R^{1a} is —S—C₁₋₆ alkyl, optionally —S-methyl or —S-ethyl. R^{1a} in each FAP-targeting group may be the same or different.

In some embodiments, at least one R^{1b} is —H. In some embodiments, at least one R^{1b} is —OH. In some embodiments, at least one R^{1b} is halogen; optionally the halogen is F, Cl, Br, or I. In some embodiments, at least one R^{1b} is C₁₋₆ alkyl, optionally methyl, ethyl, isopropyl, sec-butyl, isobutyl, or tert-butyl. In some embodiments, at least one R^{1b} is —O—C₁₋₆ alkyl, optionally —O-methyl or —O-ethyl. In some embodiments, at least one R^{1b} is —S—C₁₋₆ alkyl, optionally —S-methyl or —S-ethyl. R^{1b} in each FAP-targeting group may be the same or different.

In some embodiments, R^{1a} and R^{1b} are both hydrogens in at least one FAP-targeting group.

In some embodiments, at least one R² is —NH—. In some embodiments, at least one R² is —N(CH₃)—. In some embodiments, at least one R² is —CH₂—. In some embodiments, at least one R² is —CH(OH)—. In some embodiments, at least one R² is —CHF—. In some embodiments, at least one R² is —CF₂—. In some embodiments, at least one R² is —S—. In some embodiments, at least one R² is —O—. In some embodiments, at least one R² is CH₂, CHF, CF₂, or S. R² in each FAP-targeting group may be the same or different.

In some embodiments, R^{1a} is —H, R^{1b} is —H, and R² is CH₂, CHF, CF₂, or S in at least one FAP-targeting group. In some embodiments, R^{1a} is —H, R^{1b} is —H, and R² is —CF₂ in at least one FAP-targeting group.

In some embodiments, at least one R³ is —H. In some embodiments, at least one R³ is —CN. In some embodiments, at least one R³ is —B(OH)₂. In some embodiments, at least one R³ is —CO₂H. In some embodiments, at least one R³ is —CONH₂. In some embodiments, at least one R³ is —SO₃H. In some embodiments, at least one R³ is —SO₂NH₂. In some embodiments, at least one R³ is —PO₃H₂. In some embodiments, at least one R³ is 5-tetrazolyl. In some embodiments, at least one R³ is —CN or —B(OH)₂. R³ in each FAP-targeting group may be the same or different.

In some embodiments, R^{1a} is —H, R^{1b} is —H, R² is CH₂, CHF, CF₂, or S, and R³ is —CN or —B(OH)₂ in at least one FAP-targeting group. In some embodiments, R^{1a} is —H, R^{1b} is —H, R² is —CF₂, and R³ is —CN in at least one FAP-targeting group.

In some embodiments, at least one R⁴ is —H. In some embodiments, at least one R⁴ is methyl. In some embodiments, at least one R⁴ is ethyl. In some embodiments, at least one R⁴ is —H or methyl. R⁴ in each FAP-targeting group may be the same or different.

In some embodiments, R^{1a} is —H, R^{1b} is —H, R² is CH₂, CHF, CF₂, or S, R³ is —CN or —B(OH)₂, and R⁴ is —H or methyl in at least one FAP-targeting group. In some embodiments, R^{1a} is —H, R^{1b} is —H, R² is —CF₂, R³ is —CN, and R⁴ is —H or methyl in at least one FAP-targeting group.

In some embodiments, at least one R⁵ is ═O. In some embodiments, at least one R⁵ is ═S. R⁵ in each FAP-targeting group may be the same or different.

In some embodiments, R^{1a} is —H, R^{1b} is —H, R² is CH₂, CHF, CF₂, or S, R³ is —CN or —B(OH)₂, R⁴ is —H or methyl, and R⁵ is ═O in at least one FAP-targeting group. In some embodiments, R^{1a} is —H, Rib is —H, R² is —CF₂, R³ is —CN, R⁴ is —H or methyl, and R⁶ is ═O in at least one FAP-targeting group.

In some embodiments, at least one R⁶ is —C(O)—. In some embodiments, at least one R⁶ is —C(S)—. In some embodiments, at least one R⁶ is —O—C(O)—. In some embodiments, at least one R⁶ is —NH—C(O)—. In some embodiments, at least one R⁶ is —NH—C(S)—. R⁶ in each FAP-targeting group may be the same or different.

In some embodiments, R^{1a} is —H, R^{1b} is —H, R² is CH₂, CHF, CF₂, or S, R³ is —CN or —B(OH)₂, R⁴ is —H or methyl, R⁶ is ═O, and R⁶ is —C(O)— in at least one FAP-targeting group. In some embodiments, R^{1a} is —H, R^{1b} is —H, R² is —CF₂, R³ is —CN, R⁴ is —H or methyl, R⁶ is ═O, and R⁶ is —C(O)— in at least one FAP-targeting group.

In some embodiments, at least one n4 is 0. In some embodiments, at least one n4 is 1. In some embodiments, at least one n4 is 2. In some embodiments, at least one n4 is 3. n4 in each FAP-targeting group may be the same or different.

In some embodiments, R^{1a} is —H, R^{1b} is —H, R² is CH₂, CHF, CF₂, or S, R³ is —CN or —B(OH)₂, R⁴ is —H or methyl, R⁶ is ═O, R⁶ is —C(O)—, and n4 is 0 or 1 in at least one FAP-targeting group. In some embodiments, R^{1a} is —H, R^{1b} is —H, R² is —CF₂, R³ is —CN, R⁴ is —H or methyl, R⁶ is ═O, R⁶ is —C(O)—, and n4 is zero in at least one FAP-targeting group.

In some embodiments, at least one R⁷ is:

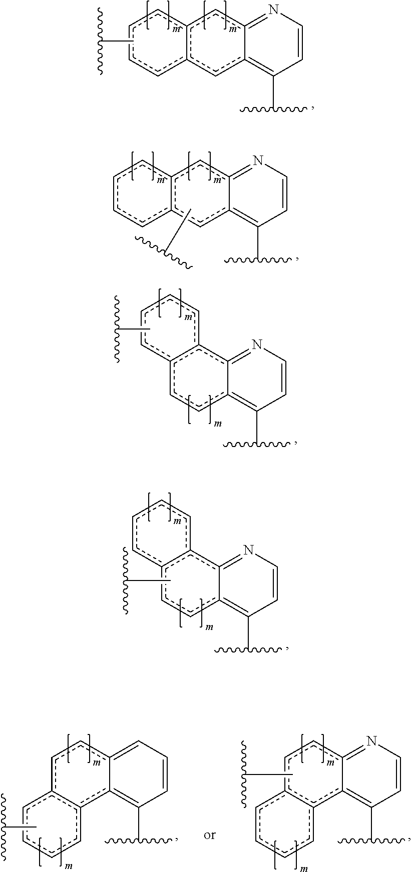

wherein the dashed bond indicates a single or double bond, wherein each m is independently 0 or 1, and the tricyclic system optionally contains 1-5 additional heteroatoms selected from N, O, and/or S, and is optionally substituted with 1-5 —OH, —NO₂, —CN, —NH₂, —NH(CH₃), NH(CH₃)₂, halogen, C₁₋₆ alkyl, —O—C₁₋₆ alkyl, or —S—C₁₋₆ alkyl. In some embodiments, the tricyclic system is fully aromatic. In some embodiments, the tricyclic system is partially aromatic. In some embodiments, one m is 0 and the other m is 1. In some embodiments, both m are 1. In some embodiments, the tricyclic system contains 1-3 additional heteroatoms. In some embodiments, the tricyclic system contains 1 additional heteroatom. In some embodiments, the additional heteroatom(s) are nitrogen. In some embodiments, the tricyclic system is not substituted. R⁷ in each FAP-targeting group may be the same or different.

In some embodiments, at least one R⁷ is:

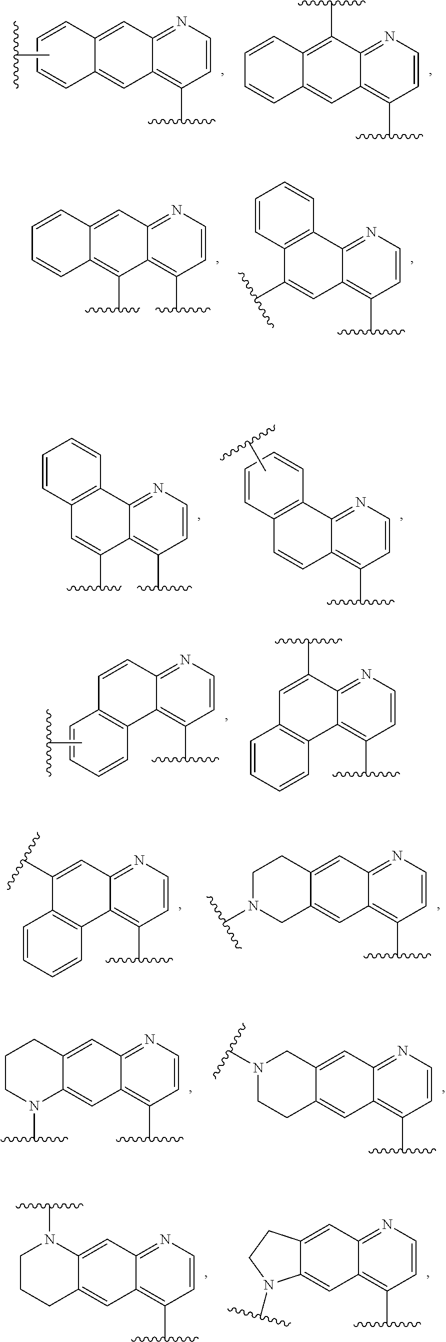

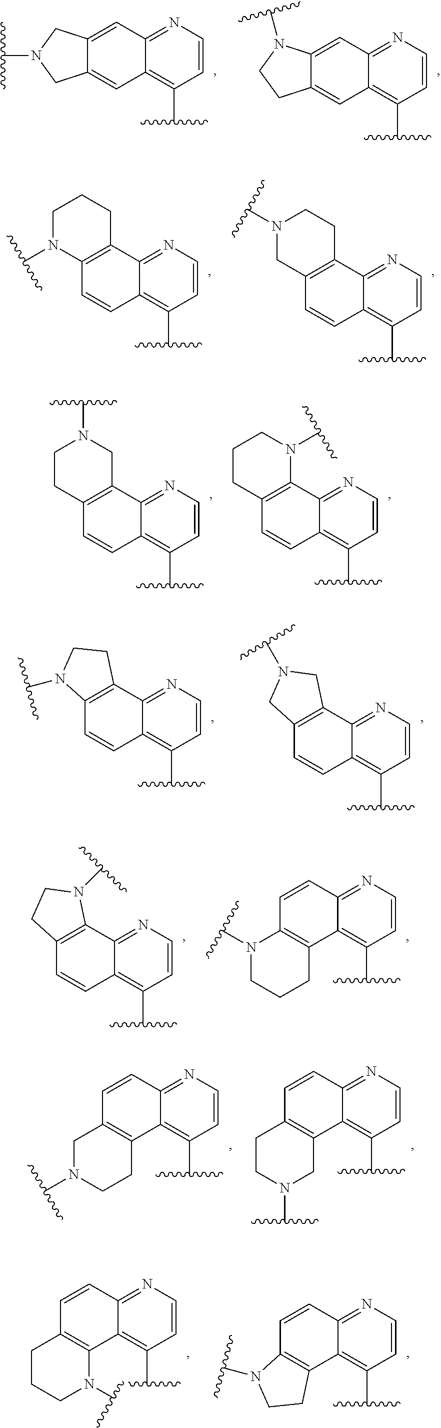

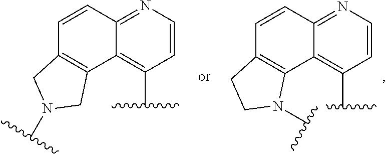 [SMILES: *c1ccnc2ccc3c(c12)CN(*)C3 ; *c1ccnc2ccc3c(c12)N(*)CC3]

wherein the tricyclic system optionally contains 1-3 additional heteroatoms selected from N, O, and/or S, and is optionally substituted with 1-5 —OH, —NO₂, —CN, —NH₂, —NH(CH₃), —N(CH₃)₂, halogen, C₁₋₆ alkyl, —O—C₁₋₆ alkyl, or —S—C₁₋₆ alkyl. In some embodiments, the tricyclic system contains 1 additional heteroatom. In some embodiments, the additional heteroatom(s) are nitrogen. In some embodiments, the tricyclic system is not substituted. R⁷ in each FAP-targeting group may be the same or different.

In some embodiments, at least one R⁷ is:

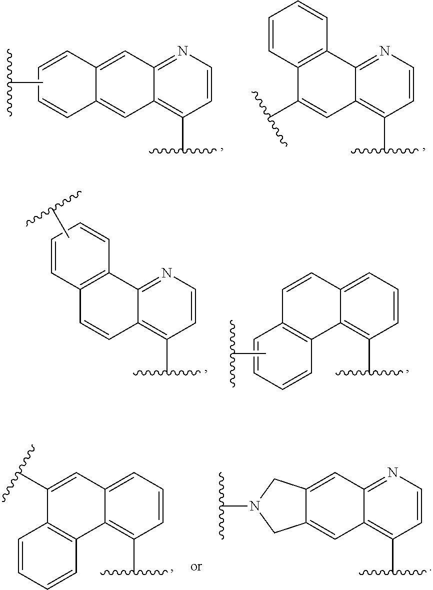

the tricyclic system is optionally contains 1-6 additional heteroatoms selected from N, O, and/or S, and is optionally substituted with 1-5 —OH, —NO₂, —CN, —NH₂, —NH(CH₃), —N(CH₃)₂, halogen, C₁₋₆ alkyl, —O—C₁₋₆ alkyl, or —S—C₁₋₆ alkyl;

Each linker (represented by R⁸) may be any suitable linker. The FAP-targeting group(s) are connected to a single linker or to multiple linkers. The radiolabeling group(s) (i.e. one or more R^{rad}) are connected to the linker or to multiple linkers. A non-limiting example of a linker is a peptide linker or a polyethylene glycol (PEG) linker.

In some embodiments, each R³ is a linear or branched chain of n5 units of R^{L1}, each unit of R^{L1} separated from each other by a unit of L¹, and each unit of R^{L1} optionally connected to an additional unit of L¹ to form a branching point, wherein each FAP-targeting group is connected to R³ through a unit of L³, and each R^{rad} is connected to R⁸ through a unit of L¹ (or may incorporate L¹ or a portion of L¹) wherein:
n5 is 1-20;
each R^{L1} is, independently, a linear, branched, and/or cyclic Cₙ₆ alkylenyl, alkenylenyl and/or alkynylenyl, wherein each n6 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
L¹ bonds to carbon, wherein each L¹ is independently —O—, —S—, —N(R^{L2})—, —N(R^{L2})C(O)—, —N(R^{L2})C(S)—, —C(O)N(R^{L2})—, —C(S)N(R^{L2})—, —NH—C(O)—NH—, —NH—C(S)—NH—,

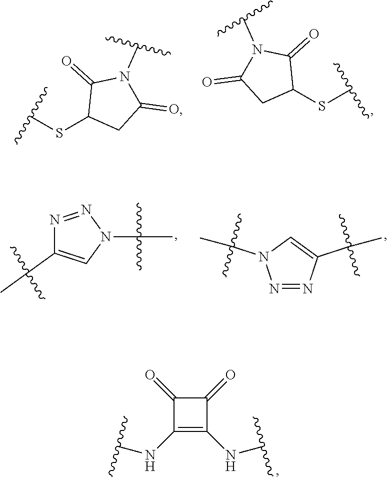

or L²;
each R^{L2} is independently H, methyl, or ethyl;
each L² is an N-containing heterocycle independently selected from the group consisting of:

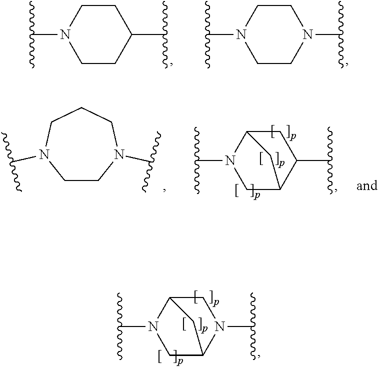

wherein each p is independently 1 or 2;
an albumin binder (R^{alb}) is optionally bonded to an L¹ of the linker (or multiple R^{alb} groups may be attached, each bonded to a separate L¹ of the linker);
each L³ is independently:
   absent, —O—, —S—, —N(R^{L2})—, —N(R^{L2})C(O)—, —N(R^{L2})C(S)—, —C(O)N(R^{L2})—, —C(S)N(R^{L2})—, —NH—C(O)—NH—, —NH—C(S)—NH—,

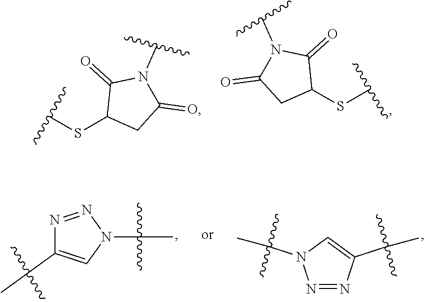

or L³ is absent, —C(O)—, —C(S)—, —N(R^{L2})C(O)—, —N(R^{L2})C(S)—, if bonded to nitrogen of the tricyclic system.

The incorporation of one or more albumin binders in the FAP-targeting compounds functions to increase plasma residence time by enhancing albumin binding (see, e.g.: Kelly, et al. 2021 Mol Imaging Biol 23, 686-696; Meng et al. 2022 J. Med. Chem. 65 (12), 8245-8257; Zhang et al. 2022 Eur J Nucl Med Mol Imaging 49, 1985-1996). In some non-limiting embodiments, each albumin binder is independently:
(CH₂)ₙ₇—CH₃ wherein n7 is 8-20;
(CH₂)ₙ₈—C(O)OH wherein n8 is 8-20;

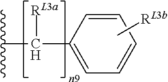

wherein n9 is 1-4 and R^{L3a} is H or methyl, and R^{L3b} is I, Br, F, Cl, H, OH, OCH₃, NH₂, NO₂ or C₁-C₆ alkyl; or

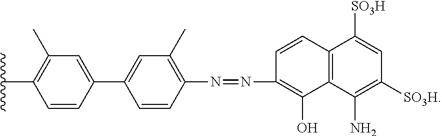 [SMILES: *c1ccc(-c2ccc(N=Nc3ccc4c(S(=O)(=O)O)cc(S(=O)(=O)O)c(N)c4c3O)c(C)c2)cc1C]

In alternative embodiments, each n5 is independently 1, 2, 3, 4, 5, 6, 7, 8, 9, 10, 11, 12, 13, 14, 15, 16, 17, 18, 19, or 20. In some embodiments, at least one n5 is 1-7. In some embodiments, at least one n5 is 1. In some embodiments, at least one n5 is 2. In some embodiments, at least one n5 is 3. In some embodiments, at least one n5 is 4. In some embodiments, at least one n5 is 5. In some embodiments, at least one n5 is 6. In some embodiments, at least one n5 is 7.

In some embodiments, R^{rad}ₙ₁—R⁸— is configured as shown in Formula III:

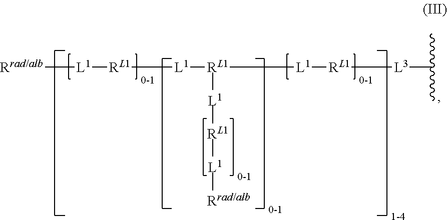

wherein L¹, R^{L1}, and L³ (bonded to R⁷ of the FAP-targeting group) are as defined above (and elsewhere in this disclosure), and R^{rad/alb} is either R^{rad} or R^{alb}, and wherein at least one R^{rad/alb} is R^{rad}, meaning the linker contains at least one R^{rad} and any remaining R^{rad/alb} may be R^{rad}, R^{alb} or a combination of R^{rad} and R^{alb}.

In some embodiments, the compound has the structure of Formula IV, or is a pharmaceutically acceptable salt thereof:

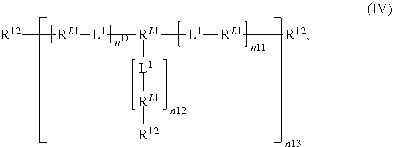

wherein:
n10, n11, and n12 are each independently 0-3;
n13 is 1-4;
each R¹² is independently -L¹-R^{rad}, -L¹-R^{alb}, or -L³-R^{FAP}, wherein at least one R¹² is -L¹-R^{rad} and at least one R¹² is -L³-R^{FAP}, optionally wherein at least one R¹² is -L¹-R^{alb}; and
R^{rad}, R^{alb}, R^{FAP}, L¹, R^{L1}, and L³ are as defined above (and elsewhere in this disclosure).

In some embodiments, n10 is 0. In other embodiments, n10 is 1. In other embodiments, n10 is 2. In other embodiments, n10 is 3.

In some embodiments, n11 is 0. In other embodiments, n11 is 1. In other embodiments, n11 is 2. In other embodiments, n11 is 3.

In some embodiments, n12 is 0. In other embodiments, n12 is 1. In other embodiments, n12 is 2. In other embodiments, n12 is 3.

In other embodiments, n13 is 1. In other embodiments, n13 is 2. In other embodiments, n13 is 3. In other embodiments, n13 is 4.

In some embodiments, the compound has the structure of Formula V, or is a pharmaceutically acceptable salt thereof:

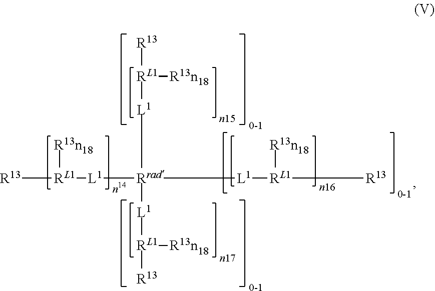

wherein:
R^{rad′} is a radiometal chelator that optionally contains multiple functional groups for attachment to a linker or linkage group (e.g. DOTA, and the like);
each R¹³ is independently -L¹-R^{rad}, -L¹-R^{alb}, or -L³-R^{FAP}, wherein at least one R¹³ is -L³-R^{FAP}, optionally wherein at least one R¹³ is -1-R^{alb}; and
n14, n15, n16, and n17 are each independently is 0-3;
each n18 is 0 or 1;
R^{rad}, R^{alb}, R^{FAP}, L¹, R^{L1}, and L³ are as defined above (and elsewhere in this disclosure).

In some embodiments of the compound of Formula V, R^{rad′} is connected to two linkers. In other embodiments, R^{rad′} is connected to three linkers. In other embodiments R^{rad′} is connected to four linkers.

In some embodiments, n14 is 0. In other embodiments, n14 is 1. In other embodiments, n14 is 2. In other embodiments, n14 is 3.

In some embodiments, n15 is 0. In other embodiments, n15 is 1. In other embodiments, n15 is 2. In other embodiments, n15 is 3.

In some embodiments, n16 is 0. In other embodiments, n16 is 1. In other embodiments, n16 is 2. In other embodiments, n16 is 3.

In some embodiments, n17 is 0. In other embodiments, n17 is 1. In other embodiments, n17 is 2. In other embodiments, n17 is 3.

Each R^{L1} (referring to any embodiment comprising R^{L1}) is, independently, a linear, branched, and/or cyclic Cₙ₆ alkylenyl, alkenylenyl and/or alkynylenyl, wherein each n6 is independently 1-20, wherei12n any carbon bonded to two other carbons is optionally independently replaced by N, S, or O, and carbons are optionally independently substituted. In some embodiments, each n6 is independently 1-15 or 1-10. In alternative embodiments, each n6 is independently 1, 2, 3, 4, 5, 6, 7, 8, 9, 10, 11, 12, 13, 14, 15, 16, 17, 18, 19, or 20. In some embodiments, each R^{L1} is independently a Cₙ₆ alkylenyl wherein any carbon bonded to two other carbons is optionally independently replaced by N, S, or O, and carbons are optionally independently substituted. In some embodiments, each R^{L1} is independently a linear C₁₋₅ alkylenyl or —(CH₂)₂—[O(CH₂)₂]₁₋₆—(CH₂)₀₋₂—; in some of these embodiments, n5 is 1-7. In some embodiments, each R^{L1} is independently-C(R^{aa})H—, wherein each R^{aa} is independently the sidechain of a proteinogenic amino acid or the sidechain of an alpha amino acid from Table 1. In some embodiments, each R¹ is independently a proteinogenic amino acid or an amino acid from Table 1 omitting the backbone amino and carboxylic acid groups of the amino acid.

Each L¹ (referring to any embodiment comprising L¹) is a linkage group. In some embodiments, at least one L¹ is —O—. In some embodiments, at least one L¹ is —S—. In some embodiments, at least one L¹ is —NH—. In some embodiments, at least one L¹ is —N(R^{L2})—; in some of these embodiments, at least one R^{L2} is methyl. In some embodiments, at least one L¹ is —N(R^{L2})C(O)—; in some of these embodiments, at least one R^{L2} is hydrogen. In some embodiments, at least one L¹ is —C(O)N(R^{L2})—; in some of these embodiments, at least one R^{L2} is hydrogen. In some embodiments, at least one L¹ is —N(R^{L2})C(S)—; in some of these embodiments, at least one R^{L2} is hydrogen. In some embodiments, at least one L¹ is —C(S)N(R^{L2})—; in some of these embodiments, at least one R^{L2} is hydrogen. In some embodiments, at least one L¹ is —NH—C(O)—NH—. In some embodiments, at least one L¹ is —NH—C(S)—NH—. In some embodiments, at least one L¹ is

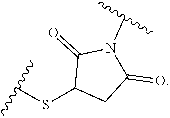 [SMILES: *SC1CC(=O)N(*)C1=O]

In some embodiments, at least one L¹ is

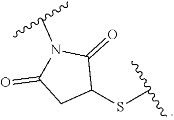 [SMILES: *SC1CC(=O)N(*)C1=O]

In some embodiments, at least one L¹ is

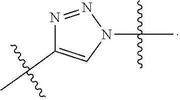 [SMILES: *c1cn(*)nn1]

In some embodiments, at least one L¹ is

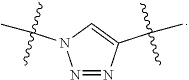 [SMILES: *c1cn(*)nn1]

In some embodiments, at least one L¹ is

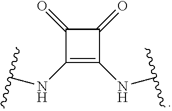 [SMILES: *Nc1c(N*)c(=O)c1=O]

In some embodiments, at least one L¹ is

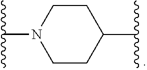 [SMILES: *C1CCN(*)CC1]

In some embodiments, at least one L¹ is

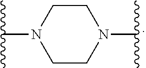 [SMILES: *N1CCN(*)CC1]

In some embodiments, at least one L¹ is

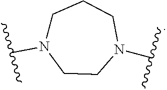 [SMILES: *N1CCCN(*)CC1]

In some embodiments, at least one L¹ is

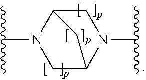

In some embodiments, at least one L¹ is

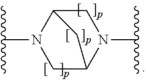

In some embodiments, at least one L¹ is

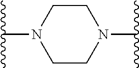 [SMILES: *N1CCN(*)CC1]

### or —C(O)—NH—.

In some embodiments, the linker has the configuration shown in Formula III, and each R^{L1} is independently a linear C₁₋₅ alkylenyl or —(CH₂)₂—[O(CH₂)₂]₁₋₆—(CH₂)₀₋₂—. In some embodiments, the compound has the configuration shown in Formula IV or V, and each R^{L1} is independently a linear C₁₋₅ alkylenyl or —(CH₂)₂—[O(CH₂)₂]₁₋₆—(CH₂)₀₋₂—.

In some embodiments, at least one R^{L1} is a linear C₁₋₅ alkylenyl. In some embodiments, at least one R^{L1} is —(CH₂)₂—[O(CH₂)₂]₁₋₆(CH₂)₀₋₂.

In some embodiments, at least one L³ is absent and R^{L1} is bonded directly to the carbon of the tricyclic system. In some embodiments, at least one L³ is —O—. In some embodiments, at least one L³ is —S—. In some embodiments, at least one L³ is —NH—. In some embodiments, at least one L³ is —N(R^{L2})—. In some embodiments, at least one L³ is —N(R^{L2})C(O)—; optionally wherein R^{L2} is methyl. In some embodiments, at least one L³ is —N(R^{L2})C(S)—. In some embodiments, at least one L³ is —C(O)N(R^{L2})—. In some embodiments, at least one L³ is —C(S)N(R^{L2})—. In some embodiments, at least one L³ is —NH—C(O)—NH—. In some embodiments, at least one L³ is —NH—C(S)—NH—. In some embodiments, at least one L³ is

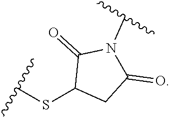 [SMILES: *SC1CC(=O)N(*)C1=O]

In some embodiments, at least one L³ is

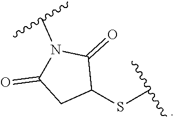 [SMILES: *SC1CC(=O)N(*)C1=O]

In some embodiments, at least one L³ is

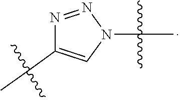 [SMILES: *c1cn(*)nn1]

In some embodiments, at least one L³ is

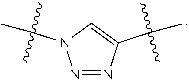 [SMILES: *c1cn(*)nn1]

In some embodiments, at least one L³ is absent and R^{L1} is bonded to nitrogen of the tricyclic system. In some embodiments, at least one L³ is —C(O)— and R^{L1} is bonded to nitrogen of the tricyclic system. In some embodiments, at least one L³ is —C(S)— and R^{L1} is bonded to nitrogen of the tricyclic system. In some embodiments, at least one L³ is —N(R^{L2})C(O)— and R^{L1} is bonded to nitrogen of the tricyclic system. In some embodiments, at least one L³ is —N(R^{L2})C(S)— and R^{L1} is bonded to nitrogen of the tricyclic system.

In some embodiments, at least one R⁸ is defined by -L¹-R^{L1}-L³- (i.e. n5 is 1). In some such embodiments, L³ is —O—. In some such embodiments, R^{L1} is a linear C₁₋₅ alkylenyl or —(CH₂)₂—[O(CH₂)₂]₁₋₆(CH₂)₀₋₂. In some such embodiments, L¹ is

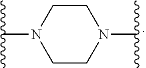 [SMILES: *N1CCN(*)CC1]

In some such embodiments,

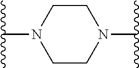 [SMILES: *N1CCN(*)CC1]

forms an amide in bonding R^{rad}

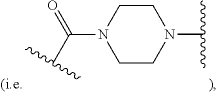 [SMILES: *C(=O)N1CCN(*)CC1]

e.g. by reacting with a carboxylic acid functional group of R^{rad}.

In some embodiments, at least one R⁸ is -L^{2′}-R^{L1′}-L^{3′}- (i.e. n5 is 1) wherein:
L^{2′} is

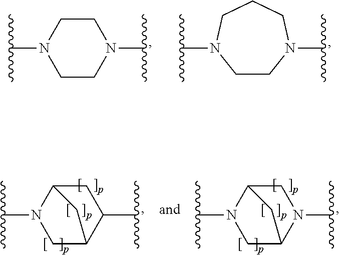

wherein each p is independently 1 or 2, optionally wherein L^{2′} is

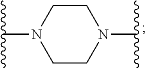 [SMILES: *N1CCN(*)CC1]

R^{L1′} is C₂₋₁₀ alkylenyl; and
L^{3′} is —O—.

In some embodiments, n1 is 1, R⁸ is -L¹-R^{L1}-L³- and forms —C(O)-Xaa¹¹- wherein Xaa¹¹ is a proteinogenic amino acid residue or an amino acid residue selected from Table 1. In some embodiments, Xaa¹¹ is pABzA-DIG. In other embodiments, Xaa¹¹ is Pip. In other embodiments, Xaa¹¹ is dPEG2. In other embodiments, Xaa¹¹ is Acp.

In some embodiments, R⁸ forms a peptide linker, wherein peptide (amide) bonds are independently optionally methylated, optionally replacing one or more amide bonds with 1,2,3-triazole linkages (product of a reaction between an azide and an alkyne). In some embodiments, the peptide linker is a linear peptide linker, optionally replacing one or more amide bonds with 1,2,3-triazole linkages. In some embodiments, the peptide linker is a branched peptide linker, where the amino acid residues may be connected through a combination of main chain amide (peptide) bonds and ‘side chain’-to-‘main chain’ or ‘side chain’-to-‘side chain’ bonds. For example, a branched peptide may be connected by one or more of: backbone (main chain) peptide (amide) bonds, ‘main chain’-to-side chain amide bonds (between an amino group and a carboxylic acid group), optionally replacing one or more amide bonds with 1,2,3-triazole linkages. In some such embodiments, the peptide linker is (Xaa¹⁰)₁₋₂₀, wherein each Xaa¹⁰ is independently a proteinogenic amino acid residue or a non-proteinogenic amino acid residue (e.g. selected from Table 1) linked together as a linear or branched peptide linker. In some embodiments, (Xaa¹⁰)₁₋₂₀ is a linear peptide linker. In some embodiments, (Xaa¹⁰)₁₋₂₀ is a branched peptide linker. R^{rad} is bonded to the peptide linker through an amide bond or another L¹ linkage group; in some embodiments, R^{rad} is bonded to the peptide linker through an amide bond.

In some embodiments, each Xaa¹⁰ is independently —N(R^{a})R^{b}C(O)— wherein: R^{a} may be H or methyl; R^{b} may be a 1-30 atom alkylenyl, heterolakylenyl, alkenylenyl, heteroalkenylenyl, alkynylenyl, or heteroalkynylenyl, including linear, branched, and/or cyclic (whether aromatic or nonaromatic as well as mono-cyclic, multicyclic or fused cyclic) structures; or N, R^{a} and R^{b} together may form a 5- to 7-atom heteroalkylenyl or heteroalkenylenyl.

In some embodiments, (Xaa¹⁰)₁₋₂₀ consists of a single amino acid or residue. In some embodiments, (Xaa¹⁰)₁₋₂₀ is a dipeptide, wherein each Xaa¹⁰ may be the same or different. In some embodiments, (Xaa¹⁰)₁₋₂₀ is a tripeptide, wherein each Xaa¹⁰ may be the same, different or a combination thereof. In some embodiments, (Xaa¹⁰)₁₋₂₀ consists of 4 amino acid residues connected by peptide bonds, wherein each Xaa¹⁰ may be the same, different or a combination thereof. In some embodiments, each Xaa¹⁰ is independently selected from proteinogenic amino acids and the non-proteinogenic amino acids listed in Table 1, wherein each peptide backbone amino group of the peptide linker is independently optionally methylated. In some embodiments, all peptide backbone amino groups of the peptide linker are methylated. In other embodiments, only one peptide backbone amino group of the peptide linker is methylated. In other embodiments, only two peptide backbone amino groups of the peptide linker are methylated. In other embodiments, no peptide backbone amino groups of the peptide linker are methylated.

In some embodiments, the linker does not comprise R^{alb}.

In some embodiments, the linker comprises R^{alb} bonded to an L¹ of the linker. In some embodiments, the linker comprises multiple R^{alb}, each bonded to a separate L¹ of the linker. In some embodiments, R⁸ comprises 1 R^{alb}. In some embodiments, the linker comprises 2 R^{alb}. In some embodiments, the linker comprises 3 R^{alb}. In some embodiments, the linker comprises 4 R^{alb}.

In some embodiments, R^{alb} is —(CH₂)ₙ₇—CH₃ wherein n7 is 8-20. In alternative embodiments, n7 is 8, 9, 10, 11, 12, 13, 14, 15, 16, 17, 18, 19, or 20.

In some embodiments, R^{alb} is —(CH₂)ₙ₈—C(O)OH wherein n8 is 8-20. In alternative embodiments, n8 is 8, 9, 10, 11, 12, 13, 14, 15, 16, 17, 18, 19, or 20.

In some embodiments, R^{alb} is

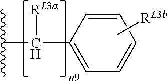

wherein n9 is 1-4 and R^{L3a} is H or methyl, and R^{L3b} is I, Br, F, Cl, H, OH, OCH₃, NH₂, NO₂ or C₁-C₆ alkyl. In alternative embodiments, n9 is 1, 2, 3, or 4. In certain embodiments, R^{L3a} is H. In certain embodiments, R^{L3a} is methyl. In certain embodiments, R^{L3b} is I, Br, F, or Cl, optionally in para position. In certain embodiments, R^{L3b} is H. In certain embodiments, R^{L3b} is OH, optionally in para position. In certain embodiments, R^{L3b} is OCH₃, optionally in para position. In certain embodiments, R^{L3b} is NH₂, optionally in para position. In certain embodiments, R^{L3b} is NO₂, optionally in para position. In certain embodiments, R^{L3b} is C₁-C₆ alkyl, optionally in para position. In certain embodiments, R^{L3a} is H and R^{L3b} is OCH₃ or NO₂. In some embodiments, R^{L3a} is methyl and R^{L3b} is isobutyl, optionally para-isobutyl.

In some embodiments, R^{alb} is

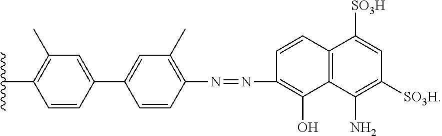 [SMILES: *c1ccc(-c2ccc(N=Nc3ccc4c(S(=O)(=O)O)cc(S(=O)(=O)O)c(N)c4c3O)c(C)c2)cc1C]

Each radiolabeling group (represented by R^{rad}) is independently: a radiometal chelator; an aryl or heteroaryl substituted with a radiohalogen; a prosthetic group containing a trihaloborate; a prosthetic group containing a silicon-halogen-acceptor moiety; or a prosthetic group containing a halophosphate, halosulfate, or sulfonyl halide. Each R^{rad} may be the same or different.

In some embodiments, at least one R^{rad} is or comprises a radiometal chelator. The radiometal chelator may be any chelator suitable for binding a radiometal, a radionuclide-bound metal, or a radionuclide-bound metal-containing prosthetic group, and which is attached to the linker by forming an amide bond (between an amino group and a carboxylic acid group) or a 1,2,3-triazole (reaction between an azide and an alkyne), or by reaction between a maleimide and a thiol group. Many suitable radiometal chelators are known, e.g. as summarized in Price and Orvig, Chem. Soc. Rev., 2014, 43, 260-290. In some embodiments, but without limitation, each radiometal chelator is independently selected from the group consisting of: DOTA and DOTA derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1 NOTAGA; EDTA; Neunpa; AlP; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide (HYNIC); dimercaptosuccinic acid; 1,2-ethylenediylbis -L-cysteine diethyl ester; methylenediphosphonate; hexamethylpropyleneamineoxime; and hexakis(methoxy isobutyl isonitrile). In some embodiments, at least one radiometal chelator is DOTA or a DOTA derivative. Each radiometal chelator may be the same or different.

Exemplary non-limiting examples of radiometal chelators and example radionuclides that may be chelated by these chelators are shown in Table 2. In alternative embodiments, at least one R^{rad} is a radiometal chelator selected from those listed above or in Table 2. It is noted, however, that one skilled in the art could replace any of the chelators listed herein with another chelator.

TABLE 2
Exemplary chelators and exemplary radionuclide which bind said chelators

| Chelator | Radionuclide |
|---|---|
| 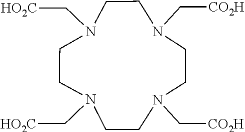 [SMILES: O=C(O)CN1CCN(CC(=O)O)CCN(CC(=O)O)CCN(CC(=O)O)CC1] DOTA, 1,4,7,10-tetraazacyclododecane- 1,4,7,10-tetraacetic acid | Cu-64/67 Ga-67/68 In-111 Lu-177 Y-86/90 Bi-203/212/213 Pb-212 Ac-225 Gd-159 Yb-175 Ho-166 As-211 Sc-44/47 Pm-149 Pr-142 Sn-117m Sm-153 Tb-149/152/155/161 Er-165 Ra-223/224 Th-227 |
| 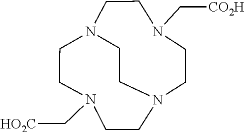 [SMILES: O=C(O)CN1CCN2CCN(CC1)CCN(CC(=O)O)CC2] CB-DO2A, 4, 10-bis(carboxymethyl)-1,4,7,10- tetraazabicyclo[5.5.2]tetradecane | Cu-64/67 |
| 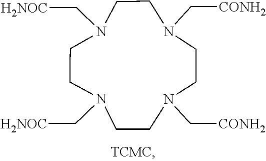 [SMILES: NC(=O)CN1CCN(CC(N)=O)CCN(CC(N)=O)CCN(CC(N)=O)CC1] 1,4,7,10-tetrakis(carbamoylmethyl)- I,4,7,10-tetraazacyclododecane | Pb-212 |
| 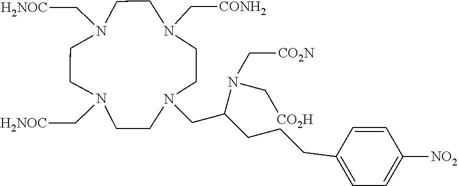 3p-C-DEPA | Bi-212/213 |
| 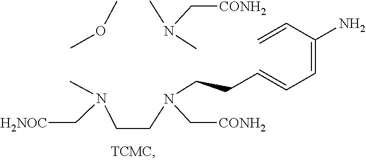 p-NH₂-Bn-Oxo-DO3A | Cu-64/67 |
| 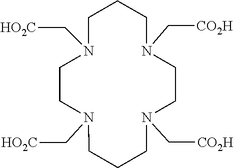 TETA, 1,4,8,11-tetraazacyclotetradecane- 1,4,8,11-tetraacetic acid | Cu-64/67 |
| 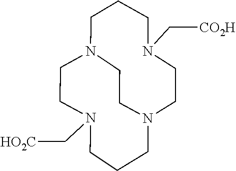 [SMILES: O=C(O)CN1CCCN2CCN(CCCN(CC(=O)O)CC2)CC1] CB-TE2A, 4, 11-bis- (carboxymethyl)-1,4,8,11- tetraazabicyclo[6.6.2]- hexadecane | Cu-64/67 |
| 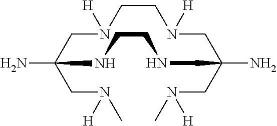 Diamsar | Cu-64/67 |
| 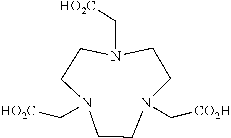 [SMILES: O=C(O)CN1CCN(CC(=O)O)CCN(CC(=O)O)CC1] NOTA, 1,4,7-triazacyclononane-1,4,7- triacetic acid | Cu-64/67 Ga-68 In-111 Sc-44/47 |
| 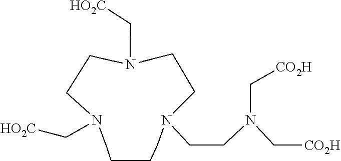 [SMILES: O=C(O)CN1CCCCN(CCN(CC(=O)O)CC(=O)O)CCN(CC(=O)O)CCC1] NETA, {4-[2-(bis-carboxymethylamino)-ethyl]- 7-carboxymethyl-[1,4,7]triazonan-1-yl}-acetic acid | Cu-64/67 Ga-68 Lu-177 Y-86/90 Bi-213 Pb-212 |
| 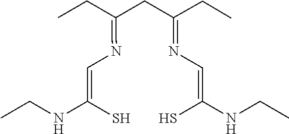 [SMILES: CCN/C(S)=C/N=C(\CC)C/C(CC)=N/C=C(\S)NCC] HxTSE | Au-198/199 |
| 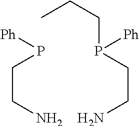 P₂N₂Ph₂ | Rh-105 |
| 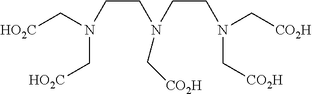 [SMILES: O=C(O)CN(CCN(CC(=O)O)CC(=O)O)CCN(CC(=O)O)CC(=O)O] DTPA, diethylenetriaminepentaacetic acid | In-111 Sc-44/47 Lu-177 Y-86/90 Sn-117m Pd-109 |
| 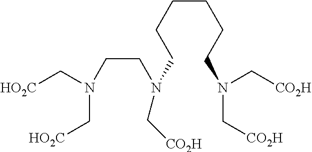 CHX-A00-DTPA, 2-(p-isothiocyanatobenzyl)- cyclohexyldiethylenetriaminepentaacetic acid | In-111 Lu-177 Y-86/90 Bi-212/213 |
| 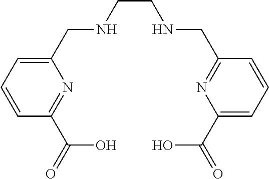 [SMILES: O=C(O)c1cccc(CNCCNCc2cccc(C(=O)O)n2)n1] H₂dedpa, 1,2-[[6-(carboxy)-pyridin-2-yl]- methylamino]ethane | Cu-64/67 |
| 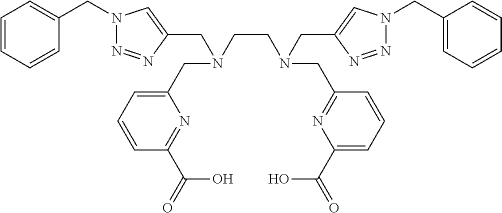 [SMILES: O=C(O)c1cccc(CN(CCN(Cc2cn(Cc3ccccc3)nn2)Cc2cccc(C(=O)O)n2)Cc2cn(Cc3ccccc3)nn2)n1] H₂azapa, N,N0-[1-benzyl-1,2,3-triazole-4-yl]methyl- N,N0-[6-(carboxy)pyridin-2-yl]-1,2-diaminoethane | Cu-64/67 |
| 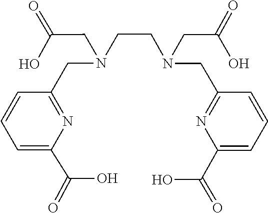 [SMILES: O=C(O)CN(CCN(CC(=O)O)Cc1cccc(C(=O)O)n1)Cc1cccc(C(=O)O)n1] H₄octapa | In-111 Lu-177 Y-86/90 Ac-225 |
| 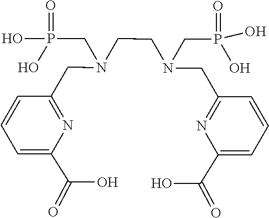 [SMILES: O=C(O)c1cccc(CN(CCN(Cc2cccc(C(=O)O)n2)CP(=O)(O)O)CP(=O)(O)O)n1] H₆phospa | Ac-225 |
| 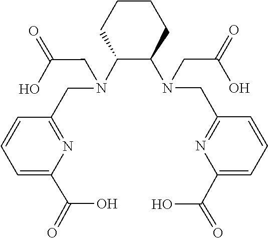 [SMILES: O=C(O)CN(Cc1cccc(C(=O)O)n1)[C@@H]1CCCC[C@H]1N(CC(=O)O)Cc1cccc(C(=O)O)n1] H₄CHXoctapa | In-111 Ac-225 |
| 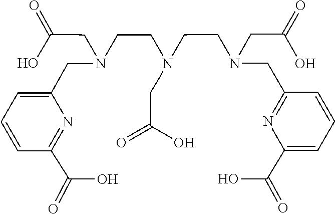 [SMILES: O=C(O)CN(CCN(CC(=O)O)Cc1cccc(C(=O)O)n1)CCN(CC(=O)O)Cc1cccc(C(=O)O)n1] H₅decapa | In-111 Lu-177 Ac-225 |
| 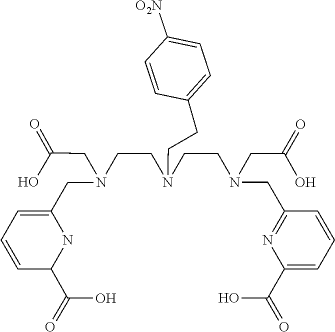 H4neunpa-p-Bn-NO₂ | In-111 Lu-177 Ac-225 |
| 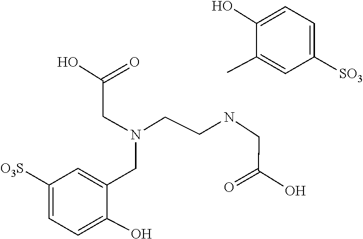 SHBED, N,N0-bis(2-hydroxy-5-sulfobenzyl)- ethylenediamine-N, NO-diacetic acid | In-111 Ga-68 |
| 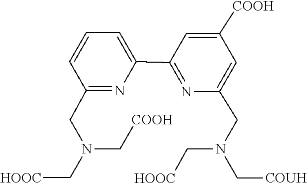 BPCA | In-111 |
| 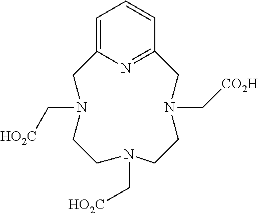 [SMILES: O=C(O)CN1CCN(CC(=O)O)Cc2cccc(n2)CN(CC(=O)O)CC1] PCTA, 3,6,9, 15-tetraazabicyclo[9.3.1]- pentadeca-1(15), 11,13-triene-3,6,9,- triacetic acid | Cu-64/67 |
| 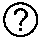 H2-MACROPA (N,N′-bis[(6-carboxy-2-pyridil)methyl]-4,13-diaza-18-crown-6) | Ac-225 |
| 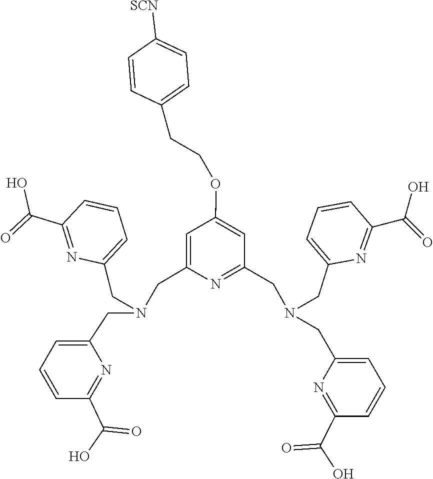 H4py4pa-phenyl-NCS | Ac-225 |
| 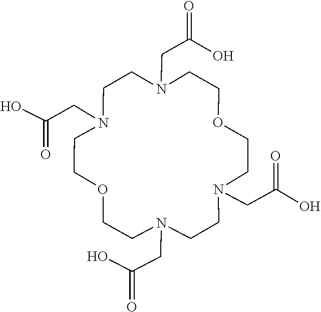 [SMILES: O=C(O)CN1CCOCCN(CC(=O)O)CCN(CC(=O)O)CCOCCN(CC(=O)O)CC1] Crown | Ac-225 |
| HYNIC | Tc-99m |
| | Tc-94m |
| 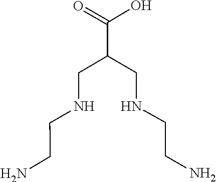 [SMILES: NCCNCC(CNCCN)C(=O)O] N4 (6-carboxy-1,4,7,11-tetraazaundecane) | Tc-99m Tc-94m |
| 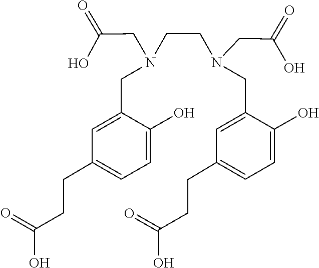 [SMILES: O=C(O)CCc1ccc(O)c(CN(CCN(CC(=O)O)Cc2cc(CCC(=O)O)ccc2O)CC(=O)O)c1] HBED-CC | Ga-68 |
| 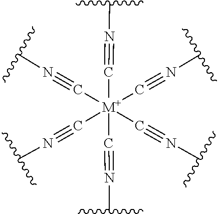 Isocyanide-coordinated metal (M+) | Tc-99m |

[symbol]

[symbol]

indicates data missing or illegible when filed

In some embodiments, each radiometal chelator is independently selected from Table 2, wherein each chelator is optionally bound by a radiometal. In some embodiments, each radiometal chelator is bound by one of the corresponding radionuclides shown in Table 2.

In some embodiments, at least one R^{rad} is DOTA, or a derivative thereof, linked via an amide (e.g. formed from one of the carboxyl groups shown in Table 2). In some embodiments, at least one R^{rad} is CB-DO2A, or a derivative thereof, linked via an amide (e.g. formed from one of the carboxyl groups shown in Table 2). In some embodiments, at least one R^{rad} is TCMC, or a derivative thereof, linked via an amide (e.g. formed from one of the —CONH₂ groups shown in Table 2). In some embodiments, the at least one R^{rad} is 3p-C-DEPA, or a derivative thereof, linked via an amide (e.g. formed from one of the carboxyl groups shown in Table 2). In some embodiments, at least one R^{rad} is p-NH₂—Bn-Oxo-DO3A or a derivative thereof, linked via an amide (e.g. formed from one of the carboxyl groups shown in Table 2). In some embodiments, at least one R^{rad} is TETA, or a derivative thereof, linked via an amide (e.g. formed from one of the carboxyl groups shown in Table 2). In some embodiments, at least one R^{rad} is CB-TE2A, or a derivative thereof, linked via an amide (e.g. formed from one of the carboxyl groups shown in Table 2). In some embodiments, at least one R^{rad} is Diamsar, or a derivative thereof, linked via an amide (e.g. formed from one of the amino groups shown in Table 2). In some embodiments, at least one R^{rad} is NOTA, or a derivative thereof, linked via an amide (e.g. formed from one of the carboxyl groups shown in Table 2). In some embodiments, at least one R^{rad} is NETA, or a derivative thereof, linked via an amide (e.g. formed from one of the carboxyl groups shown in Table 2). In some embodiments, at least one R^{rad} is HxTSE, or a derivative thereof, linked via an amide (e.g. formed from one of the amino groups shown in Table 2). In some embodiments, at least one R^{rad} is P₂N₂Ph₂, or a derivative thereof, linked via an amide (e.g. formed from one of the amino groups shown in Table 2). In some embodiments, at least one R^{rad} is DTPA, or a derivative thereof, linked via an amide (e.g. formed from one of the carboxyl groups shown in Table 2). In some embodiments, at least one R^{rad} is CHX-A00-DTPA, or a derivative thereof, linked via an amide (e.g. formed from one of the carboxyl groups shown in Table 2). In some embodiments, at least one R^{rad} is H₂dedpa, or a derivative thereof, linked via an amide (e.g. formed from one of the carboxyl groups shown in Table 2). In some embodiments, at least one R^{rad} is H₂azapa, or a derivative thereof, linked via an amide (e.g. formed from one of the carboxyl groups shown in Table 2). In some embodiments, at least one R^{rad} is H₄octapa, or a derivative thereof, linked via an amide (e.g. formed from one of the carboxyl groups shown in Table 2). In some embodiments, at least one R^{rad} is Hephospa, or a derivative thereof, linked via an amide (e.g. formed from one of the carboxyl groups shown in Table 2). In some embodiments, at least one R^{rad} is H₄CHXoctapa, or a derivative thereof, linked via an amide (e.g. formed from one of the carboxyl groups shown in Table 2). In some embodiments, at least one R^{rad} is H₅decapa, or a derivative thereof, linked via an amide (e.g. formed from one of the carboxyl groups shown in Table 2). In some embodiments, at least one R^{rad} is H₄neunpa-p-Bn-NO2, or a derivative thereof, linked via an amide (e.g. formed from one of the carboxyl groups shown in Table 2). In some embodiments, at least one R^{rad} is SHBED, or a derivative thereof, linked via an amide (e.g. formed from one of the carboxyl groups shown in Table 2). In some embodiments, at least one R^{rad} is BPCA, or a derivative thereof, linked via an amide (e.g. formed from one of the carboxyl groups shown in Table 2). In some embodiments, at least one R^{rad} is PCTA, or a derivative thereof, linked via an amide (e.g. formed from one of the carboxyl groups shown in Table 2). In some embodiments, at least one R^{rad} is H2-MACROPA, or a derivative thereof, linked via an amide (e.g. formed from one of the carboxyl groups shown in Table 2). In some embodiments, at least one R^{rad} is Crown, or a derivative thereof, linked via an amide (e.g. formed from one of the carboxyl groups shown in Table 2). In some embodiments, at least one R^{rad} is HYNIC, or a derivative thereof, linked via an amide (e.g. formed from the carboxyl group shown in Table 2). In some embodiments, at least one R^{rad} is N4, or a derivative thereof, linked via an amide (e.g. formed from the carboxyl group shown in Table 2). In some embodiments, at least one R^{rad} is HBED-CC, or a derivative thereof, linked via an amide (e.g. formed from one of the carboxyl groups shown in Table 2). In some embodiments, R^{rad} is a hexamer of isocyanate coordinated to a metal ion, optionally ^{99m}Tc; in such compounds, n1 is 1, n2 is 6, and n3 is 6 (e.g. see Ruan et al. 2022 Molecular Pharmaceutics 19(1), 160-171).

In some embodiments, the radiometal chelator (or one of the radiometal chelators) is a derivative of a radiometal chelator shown in Table 2. A derivative may include, e.g. (1) modification of a functional group of the chelator (e.g. a carboxyl group, an amino group, etc.) or (2) attachment of a new functional group (e.g. attachment of an R-group to an ethylene carbon located between two nitrogen atoms, wherein the R-group is a functional group fused to a spacer). In some embodiments, a carboxyl functional group shown in Table 2 is replaced with azidopropyl ethylacetamide (e.g. azido-mono-amide-DOTA), butynylacetamide (e.g. butyne-DOTA), thioethylacetamide (e.g. DO3A-thiol), maleimidoethylacetamide (e.g. maleimido-mono-amide-DOTA), or N-hydroxysuccinimide ester (e.g. DOTA-NHS-ester). When linked, these derivative chelators can be linked either via an amide (formed from a remaining carboxyl group) or via —C(O)—NH—(CH₂)₂₋₃-(triazole) or —C(O)—NH—(CH₂)₂₋₃-(thiomaleimide). In other embodiments, a backbone carbon (e.g. in an ethylene positioned between two backbone nitrogen atoms) in the chelator ring is fused to an R-group containing a functional group, optionally wherein the R-group is —(CH₂)₁₋₃-(phenyl)-N═C═S or —(CH₂)₁₋₃-(phenyl)-N═C═O, optionally 1,4-isothiocyanatobenzyl; e.g. p-SCN-Bn-DOTA (S-2-(4-isothiocyanatobenzyl)-1,4,7,10-tetraazacyclododecane tetraacetic acid), p-SCN-Bn-NOTA (2-S-(4-isothiocyanatobenzyl)-1,4,7-triazacyclononane-1,4,7-triacetic acid), and the like. When linked, these derivatives can form a urea linkage (formed from isocyanate) or a thiourea linkage (formed from isothiocyanate).

Since various radiometal chelators have multiple functional groups for attaching a linker (e.g. DOTA, isocyanide coordinated metal ion, and the like), a single radiometal chelator may be bonded to multiple linkers, each linker bonded to a FAP-targeting group, to produce a dimer, trimer, tetramer, or even hexamer.

In some embodiments, a radiometal chelator is conjugated with a radiometal, a radionuclide-bound metal, or a radionuclide-bound metal-containing prosthetic group, and the radiometal, the radionuclide-bound metal, or the radionuclide-bound metal-containing prosthetic group is chelated to the radionuclide-chelator complex. In some embodiments, the radiometal, the radionuclide-bound metal, or the radionuclide-bound metal-containing prosthetic group is: ⁶⁸Ga, ⁶¹CU, ⁶⁴CU, ⁶⁷CU, ⁶⁷Ga, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, or [¹⁸F]AlF. In other embodiments, the radiometal, the radionuclide-bound metal, or the radionuclide-bound metal-containing prosthetic group is: ⁶⁸Ga, ⁶¹CU, ⁶⁴CU, ⁶⁷CU, ⁶⁷Ga, ¹¹¹In, ⁴⁴Sc, ⁸⁶Y, ¹⁷⁷Lu, ⁹⁰Y, ²²⁵Ac, ²¹³Bi, or ²¹²Bi. In some embodiments, the chelator is a chelator from Table 2 and the chelated radionuclide is a radionuclide indicated in Table 2 as a binder of the chelator.

In some embodiments, the chelator is: DOTA or a derivative thereof, conjugated with ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu or ⁶⁷Cu; H2-MACROPA conjugated with ²²⁵Ac; Me-3,2-HOPO conjugated with ²²⁷Th; H₄py4pa conjugated with ²²⁵Ac, ²²⁷Th or ¹⁷⁷Lu; H₄pypa conjugated with ¹⁷⁷Lu; NODAGA conjugated with ⁶⁸Ga; DTPA conjugated with ¹¹¹In; or DFO conjugated with ⁸⁹Zr.

In some embodiments, the chelator is TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), TRAP (1,4,7-triazacyclononane-1,4,7-tris[methyl(2-carboxyethyl)phosphinic acid), HBED (N,N0-bis(2-hydroxybenzyl)-ethylenediamine-N,N0-diacetic acid), 2,3-HOPO (3-hydroxypyridin-2-one), PCTA (3,6,9,15-tetraazabicyclo[9.3.1]-pentadeca-1(15),11,13-triene-3,6,9,-triacetic acid), DFO (desferrioxamine), DTPA (diethylenetriaminepentaacetic acid), OCTAPA (N,N0-bis(6-carboxy-2-pyridylmethyl)-ethylenediamine-N,N0-diacetic acid) or another picolinic acid derivative.

In some embodiments, an R^{rad} is a chelator for radiolabelling with ^{99m}Tc, ^{94m}Tc, ¹⁸⁶Re, or ¹⁸⁸Re, such as mercaptoacetyl, hydrazinonicotinamide, dimercaptosuccinic acid, 1,2-ethylenediylbis -L-cysteine diethyl ester, methylenediphosphonate, hexamethylpropyleneamineoxime and hexakis(methoxy isobutyl isonitrile), and the like. In some embodiments, an R^{rad} is a chelator, wherein the chelator is mercaptoacetyl, hydrazinonicotinamide, dimercaptosuccinic acid, 1,2-ethylenediylbis -L-cysteine diethyl ester, methylenediphosphonate, hexamethylpropyleneamineoxime or hexakis(methoxy isobutyl isonitrile). In some of these embodiments, the chelator is bound by a radionuclide. In some such embodiments, the radionuclide is ^{99m}Tc, ^{94m}Tc, ¹⁸⁶Re, or ¹⁸⁸Re.

In some embodiments, an R^{rad} is a chelator that can bind ¹⁸F-aluminum fluoride ([¹⁸F]AlF), such as 1,4,7-triazacyclononane-1,4-diacetate (NODA) and the like. In some embodiments, the chelator is NODA. In some embodiments, the chelator is bound by [¹⁸F]AlF.

In some embodiments, an R^{rad} is a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

In certain embodiments, at least one R^{rad} is a prosthetic group containing a trifluoroborate (BF₃), capable of ¹⁸F/¹⁹F exchange radiolabeling. In some of these embodiments, the prosthetic group is R¹¹—R¹⁰—, wherein R¹⁰ is —(CH₂)₁₋₅— (optionally methylene), and wherein R¹¹ is

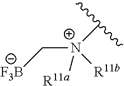

wherein R^{11a} and R^{11b} are each independently a C₁-C₅ linear or branched alkyl group, or R¹¹ is a structure listed in Table 3 (below) or Table 4 (below). For Tables 3 and 4, each R group in each pyridine substituted with —OR, —SR, —NR—, —NHR or —NR₂ is independently a C₁-C₅ linear or branched alkyl. In some embodiments, at least one R¹¹ is

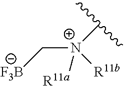

wherein R^{11a} and R^{11b} are each independently a C₁-C₅ linear or branched alkyl group. In some embodiments, at least one of the R¹¹ group(s) is/are selected from those listed in Table 3. In some embodiments, at least one of the R¹¹ group(s) is/are selected from those listed in Table 4. The trifluoroborate-containing prosthetic group(s) may comprise ¹⁸F. In some embodiments, one fluorine in BF₃ forms is ¹⁸F. In some embodiments, all three fluorines in BF₃ are ¹⁸F. In some embodiments, all three fluorines in BF₃ are ¹⁹F. Where multiple BF3-containing prosthetic groups are present in the compound, each R¹¹ may be independently selected from any of those disclosed herein.

TABLE 3
Exemplary R¹¹ groups.

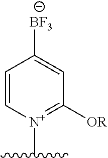

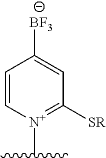

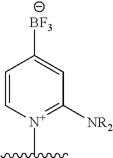

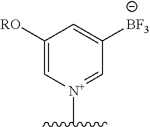

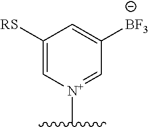

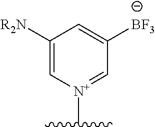

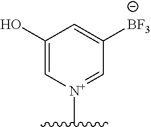

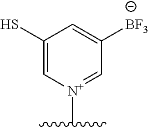

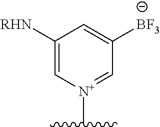

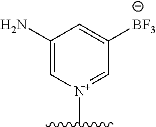

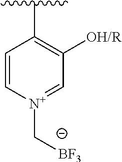

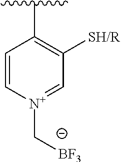

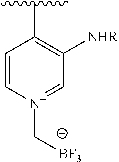

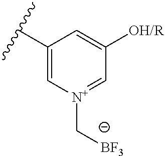

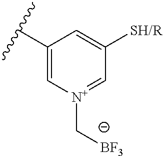

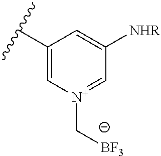

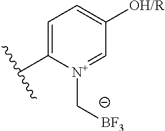

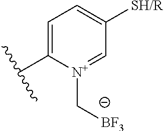

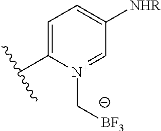

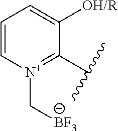

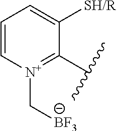

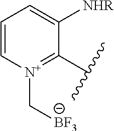

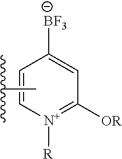

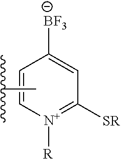

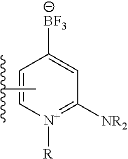

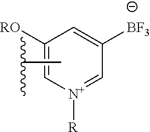

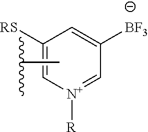

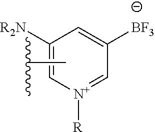

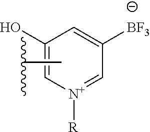

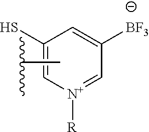

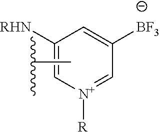

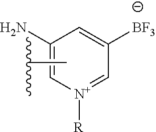

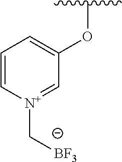

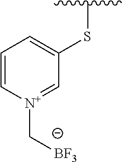

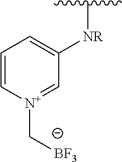

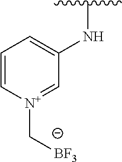

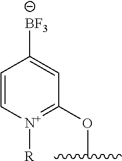

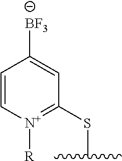

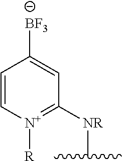

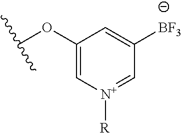

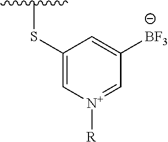

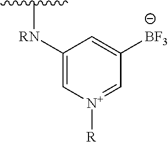

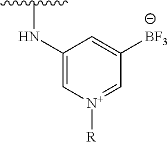

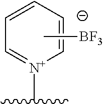

TABLE 4
Exemplary R¹¹ groups.

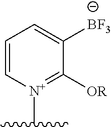

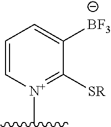

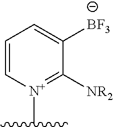

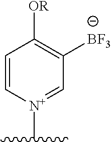

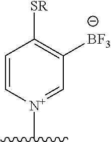

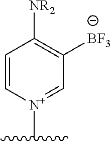

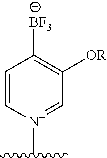

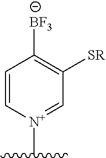

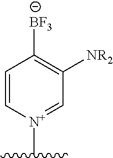

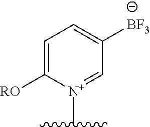

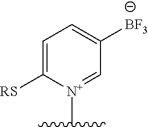

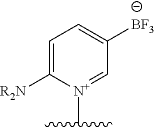

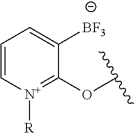

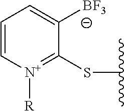

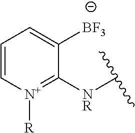

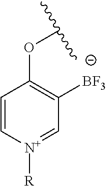

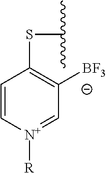

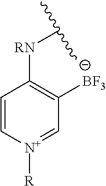

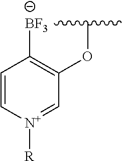

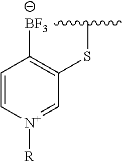

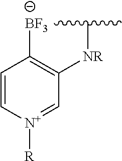

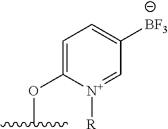

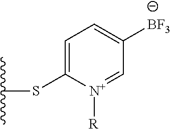

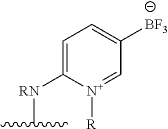

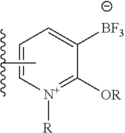

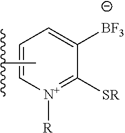

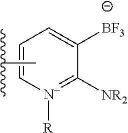

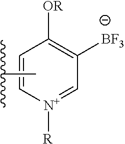

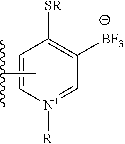

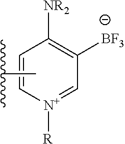

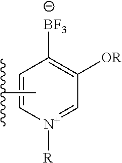

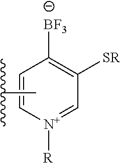

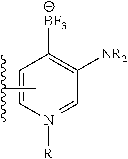

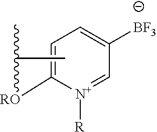

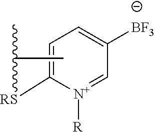

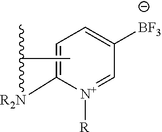

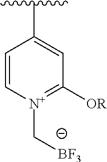

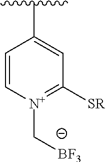

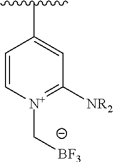

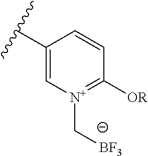

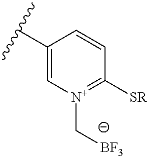

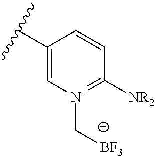

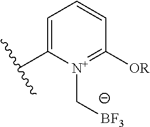

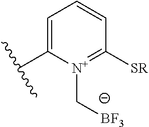

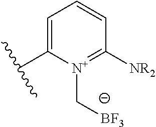

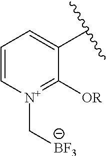

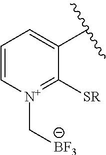

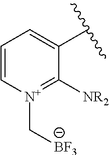

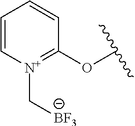

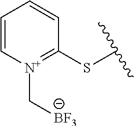

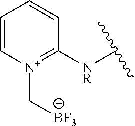

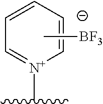

In some embodiments, an R¹¹ may independently form

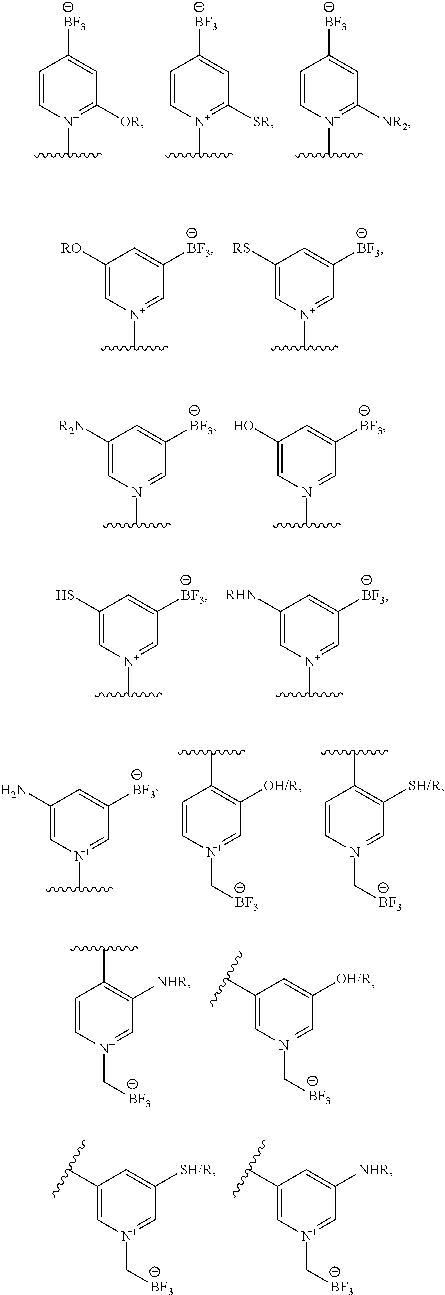

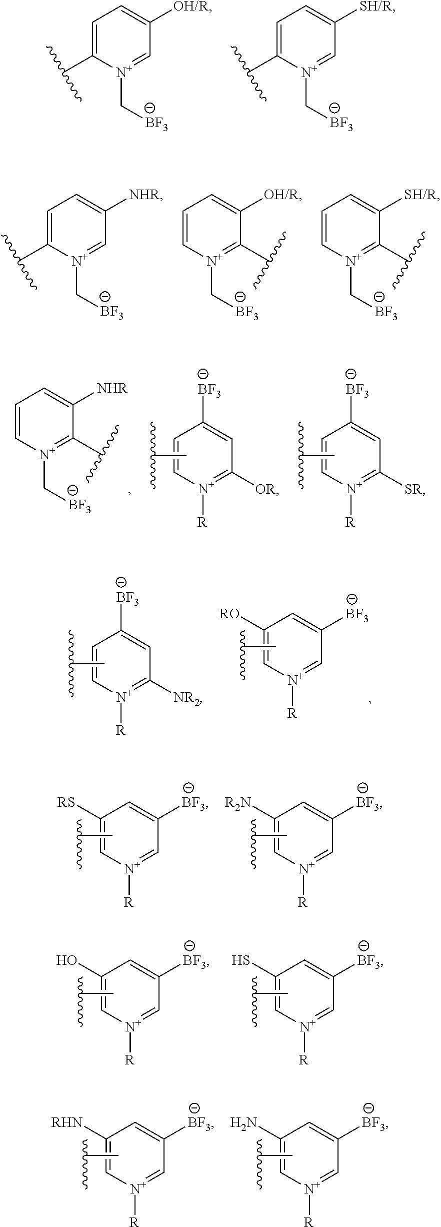

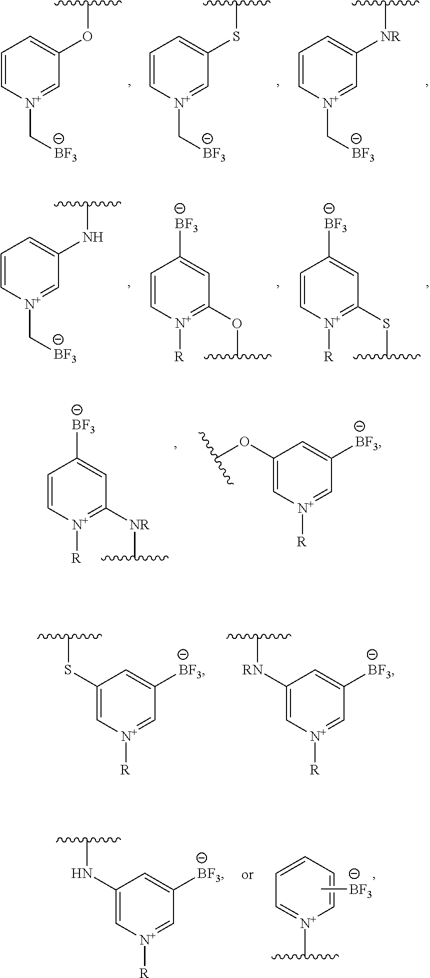

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. The trifluoroborate-containing prosthetic group(s) may comprise ¹⁸F. In some embodiments, one fluorine is an R¹¹ is ¹⁸F. In some embodiments, all three fluorines in an R¹¹ are ¹⁸F. In some embodiments, all three fluorines in an R¹¹ are ¹⁹F.

In some embodiments, an R¹¹ may independently form,

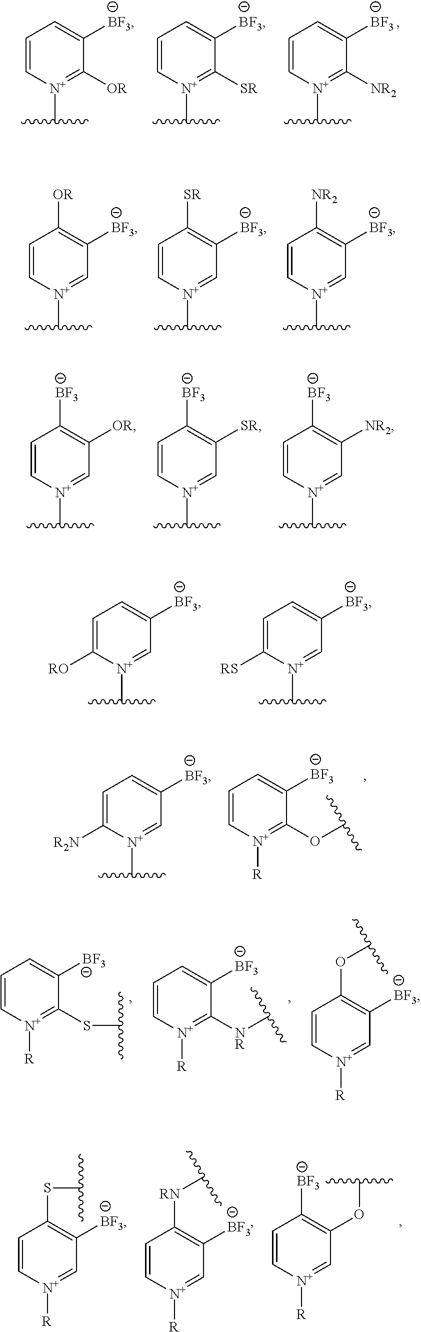

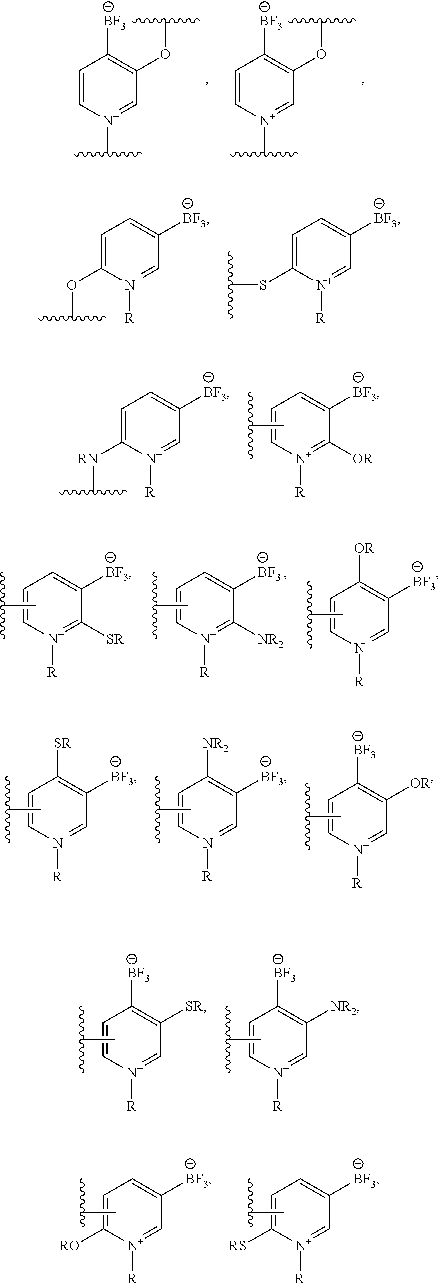

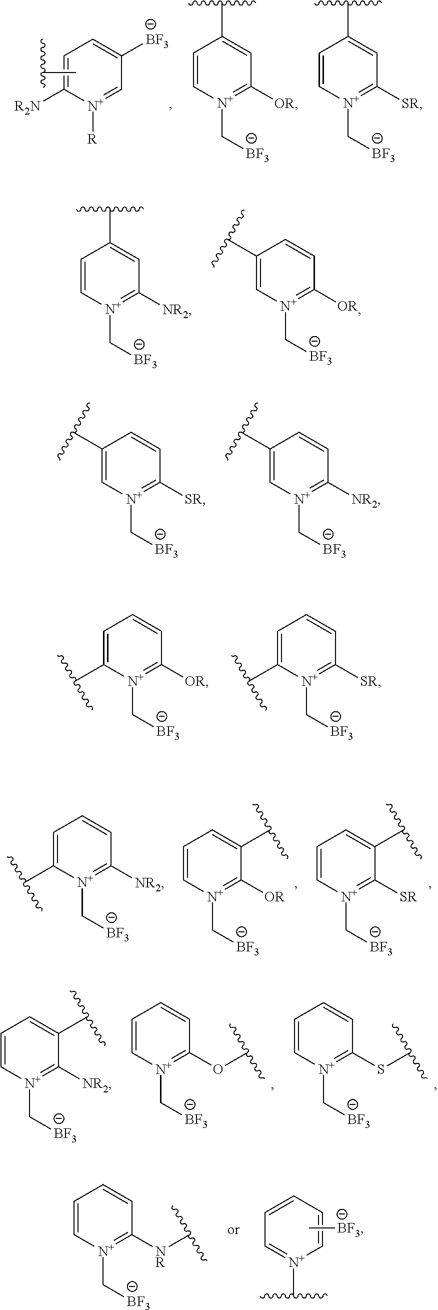

in which each R (when present) in the pyridine substituted —OR, —SR, —NR—, —NHR or —NR₂ is independently a linear or branched C₁-C₅ alkyl. In some embodiments, R is methyl. In some embodiments, R is ethyl. In some embodiments, R is propyl. In some embodiments, R is isopropyl. In some embodiments, R is n-butyl. In some embodiments, an R¹¹ is

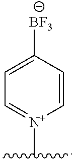

In some embodiments, all three fluorines in an R¹¹ are ¹⁸F. In some embodiments, one fluorine in an R¹¹ is ¹⁸F. In some embodiments, all three fluorines in an R¹¹ are ¹⁹F.

In some embodiments, at least one R¹¹ or optionally each R¹¹ is independently

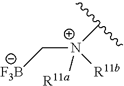

wherein R^{11a} and R^{11b} are each independently a C₁-C₅ linear or branched alkyl group. In some embodiments, R^{11a} is methyl. In some embodiments, R^{11a} is ethyl. In some embodiments, R^{11a} is propyl. In some embodiments, R^{11a} is isopropyl. In some embodiments, R^{11a} is butyl. In some embodiments, R^{11a} is n-butyl. In some embodiments, R^{11a} is pentyl. In some embodiments, R^{11b} is methyl. In some embodiments, R^{11b} is ethyl. In some embodiments, R^{11b} is propyl. In some embodiments, R^{11b} is isopropyl. In some embodiments, R^{11b} is butyl. In some embodiments, R^{11b} is n-butyl. In some embodiments, R^{11b} is pentyl. In some embodiments, R^{11a} and R^{11b} are both methyl. The trifluoroborate-containing prosthetic group may comprise ¹⁸F. In some embodiments, one fluorine in R¹¹ is ¹⁸F. In some embodiments, all three fluorines in R¹¹ are ¹⁸F. In some embodiments, all three fluorines in R¹¹ are ¹⁹F.

In certain embodiments, the compound is conjugated with a radionuclide for positron emission tomography (PET) or single photon emission computed tomography (SPECT) imaging of GRPR expressing tumors, wherein the compound is conjugated with a radionuclide that is a positron emitter or a gamma emitter. In some embodiments, the positron or gamma emitting radionuclide is ⁶⁸Ga, ⁶⁷Ga, ⁶¹Cu, ⁶⁴Cu, ^{94m}Tc, ^{99m}Tc, ¹⁰⁵Rh, ¹¹⁰In, ¹¹¹In, ⁴⁴Sc, ⁸⁶Y, ⁸⁹Zr, ⁹⁰Nb, ¹⁵²Tb, ¹⁵⁵Tb, ²⁰³Pb, ¹⁸F, ¹³¹I, ¹²³I, ¹²⁴I or ⁷²As.

In certain embodiments the compound is conjugated with a radionuclide that is used for therapy. In some embodiments, the therapeutic radionuclide is ¹⁶⁵Er, ²¹²Bi, ²¹¹At, ¹⁶⁶Ho, ¹⁴⁹Pm ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²¹²Pb, ⁴⁷Sc, ⁹⁰Y, ²²⁵Ac, ^{117m}Sn, ¹⁵³Sm, ¹⁴⁹Tb, ¹⁶¹Tb, ²²⁴Ra, ²¹²Bi, ²²⁷Th, ²²³Ra, ¹⁸⁸Re, ¹⁸⁶Re, ²¹¹At, ¹³¹I, ⁶⁴Cu, or ⁶⁷Cu.

In some embodiments, the compound is SB03178, optionally conjugated by a radiometal. In some embodiments, the compound is SB04033, optionally conjugated by a radiometal. In alternative embodiments, the radiometal is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²¹²Pb, ²²⁵Ac, ²²⁷Th, ²²³Ra, ⁴⁷Sc, ⁶⁴Cu, or ⁶⁷Cu. In some embodiments, the radiometal is ⁶⁸Ga. In some embodiments, the radiometal is ⁶⁴Cu. In some embodiments, the radiometal is ⁶⁷Cu. In some embodiments, the radiometal is ⁶⁷Ga. In some embodiments, the radiometal is ¹¹¹In. In some embodiments, the radiometal is ¹⁷⁷Lu. In some embodiments, the radiometal is ⁹⁰Y In some embodiments, the radiometal is ²²⁵Ac.

When a radiolabeling group (i.e. R^{rad}) of the compound comprises or is conjugated to a diagnostic radionuclide, there is disclosed use of certain embodiments of a compound as disclosed herein for preparation of a radiolabelled tracer for imaging FAP-expressing tissues in a subject. There is also disclosed a method of imaging FAP-expressing tissues in a subject, in which the method comprises: administering to the subject a composition comprising certain embodiments of the compound and a pharmaceutically acceptable excipient; and imaging tissue of the subject, e.g. using PET or SPECT. When the tissue is a diseased tissue (e.g. a FAP-expressing cancer), FAP-targeted treatment may then be selected for treating the subject.

When a radiolabeling group (i.e. R^{rad}) of the compound comprises or is conjugated to a therapeutic radionuclide, there is disclosed use of certain embodiments of the compound (or a pharmaceutical composition thereof) for the treatment of FAP-expressing conditions or diseases (e.g. cancer and the like) in a subject. Accordingly, there is provided use of a compound disclosed herein in preparation of a medicament for treating a FAP-expressing condition or disease in a subject. There is also provided a method of treating FAP-expressing disease in a subject, in which the method comprises: administering to the subject a composition comprising the compound and a pharmaceutically acceptable excipient. For example, but without limitation, the disease may be a FAP-expressing cancer.

The compound may comprise both a diagnostic radionuclide and a therapeutic radionuclide.

FAP has been identified as a target for imaging and treating a variety of diseases or conditions. In some imaging/diagnostic embodiments, the FAP-expressing condition or disease is one or any combination of two or more of the following: reparative fibrosis (e.g. following myocardial infarction), arthritis, fibrosis (e.g. liver fibrosis, renal fibrosis), atherosclerosis, cirrhosis, inflammation (e.g. pancreatic lesions, Crohn's disease, gastrointestinal inflammation, eosinophilic enteritis, inflammatory bowel disease), tuberculous lesions, tubercular meningitis, FAP-expressing cancer such as renal cell cancer, insulinoma, neuroendocrine prostate cancer, thyroid cancer, pheochromocytoma, adenoid cystic cancer, gastric cancer, hepatocellular carcinoma, cervical cancer, medullary thyroid cancer, small intestine cancer, neuroendocrine tumor, anal cancer, colorectal cancer, chordoma, desmoid, ovarian cancer, head and neck cancer, thymus cancer, pancreatic cancer, prostate cancer, lung cancer, breast cancer, cholangiocellular carcinoma, esophageal cancer, salivary gland cancer, sarcoma, carcinoma of unknown primary, glioma, epithelial carcinoma, bone sarcoma, soft tissue sarcoma, melanoma, and/or astrocytoma (e.g. IDH-wildtype glioblastomas and grade Ill/IV), large B cell lymphoma, and gastric lymphoma (Qiao, et al. Mol. Pharmaceutics 2022 19 (11), 4171-4178; Langer, et al. Theranostics 2021 11(16): 7755-7766; Xu, et al. Eur J Nucl Med Mol Imaging 2021 48:1254-1255; Pirasteh, et al. Journal of Nuclear Medicine April 2022, jnumed.121.263736; Zhou, et al. [⁶⁸Ga]Ga-DOTA-FAPI-04 PET/CT imaging of cirrhosis with hepatocellular carcinoma Eur J Nucl Med Mol Imaging 12 Dec. 2022 https://doi.org/10.1007/s00259-022-06077-0; Zhou, et al. Eur J Nucl Med Mol Imaging 2021 48:3493-3501; Lou, et al. Eur J Nucl Med Mol Imaging 2019 46:2625-2626; Luo, et al. Eur J Nucl Med Mol Imaging 2021 48:1682-1683; Fu and Zhou Active uptake of [¹⁸F]F-FAPI-42 in eosinophilic gastrointestinal disorder Eur J Nucl Med Mol Imaging 1 Dec. 2022 https://doi.org/10.1007/s00259-022-06055-6; Hao, et al. Eur J Nucl Med Mol Imaging 2021 48: 651-652; Wu, et al. J Nucl Med 2022 63:948-951; Meletta, et al. Molecules 2015, 20, 2081-2099; Kratochwil, et al. J Nucl Med 2019 60:801-805; Röhrich, et al. Eur J Nucl Med Mol Imaging 2019 46:2569-2580; Wang, et al. Eur J Nucl Med Mol Imaging 2021 48:647-648; Shi, et al. Eur J Nucl Med Mol Imaging 2021 48:196-203). In some therapeutic embodiments, the FAP-expressing condition or disease is one or any combination of two or more of the following: renal cell cancer, insulinoma, neuroendocrine prostate cancer, thyroid cancer, pheochromocytoma, adenoid cystic cancer, gastric cancer, hepatocellular carcinoma, cervical cancer, medullary thyroid cancer, small intestine cancer, neuroendocrine tumor, anal cancer, colorectal cancer, chordoma, desmoid, ovarian cancer, head and neck cancer, thymus cancer, pancreatic cancer, prostate cancer, lung cancer, breast cancer, cholangiocellular carcinoma, esophageal cancer, salivary gland cancer, sarcoma, carcinoma of unknown primary, glioma, epithelial carcinoma, bone sarcoma, soft tissue sarcoma, melanoma, and/or astrocytoma (e.g. IDH-wildtype glioblastomas and grade Ill/IV), large B cell lymphoma, and gastric lymphoma (e.g. gastric diffuse large B cell lymphoma), hepatic carcinoma (Kratochwil, et al. J Nucl Med 2019 60:801-805; Röhrich, et al. Eur J Nucl Med Mol Imaging 2019 46:2569-2580; Wang, et al. Eur J Nucl Med Mol Imaging 2021 48:647-648; Shi, et al. Eur J Nucl Med Mol Imaging 2021 48:196-203).

The present invention will be further illustrated in the following examples.

### EXAMPLES

### Synthetic Methods

The compounds presented herein may incorporate peptide linkers, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g. under mild basic conditions, such as piperidine (20-50% v/v) in DMF. The amino acid to be added must also have been activated for coupling (e.g. at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1, 1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like.

Apart from forming typical peptide bonds to elongate a peptide, peptides may be elongated in a branched fashion by attaching to side chain functional groups (e.g. carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF. Deprotected side chains of Asp/Glu (L- or D-forms) and Lys/Orn/Dab/Dap (L- or D-forms) can then be coupled, e.g. by using the coupling reaction conditions described above. The above provides means for including multiple BF₃ groups.

Peptide backbone amides may be N-methylated (i.e. alpha amino methylated) or N-alkylated. This may be achieved by directly using Fmoc-N-methylated (or Fmoc-N-alkylated) amino acids during peptide synthesis. Alternatively, N-methylation under Mitsunobu conditions may be performed. First, a free primary amine group is protected using a solution of 4-nitrobenzenesulfonyl chloride (Ns-Cl) and 2,4,6-trimethylpyridine (collidine) in NMP. N-methylation (or N-alkylation) may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated (or N-alkylated) alpha amino groups, HATU, HOAt and DIEA may be used.

The formation of the thioether (—S—) linkages (e.g. for L¹) can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino)propyl bromide and the like) in an appropriate solvent (such as N,N-dimethylformamide and the like) in the presence of base (such as N,N-diisopropylethylamine and the like). If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

The formation of the linkage (e.g. for L¹) between a thiol group and a maleimide group can be performed using the conditions described above for the formation of the thioether (—S—) linkage simply by replacing the alkyl halide with a maleimide-containing compounds. Similarly, this reaction can be conducted in solid phase or solution phase. If the reactions are carried out in solution phase, the reactants used are preferably in equivalent molar ratio (1 to 1), and the desired products can be purified by flash column chromatography or high performance liquid chromatography (H PLC). If the reactions are carried out on solid phase, meaning one reactant has been attached to a solid phase, then the other reactant is normally used in excess amount (3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

Urea or thiourea linkages can be made from reaction of an amine group with an isocyanate or an isothiocyanate, respectively, which are common functional groups on radiometal chelators. The isothiocyanate functional group may be added to the radiometal chelator by reacting an amino group on the chelator with thiophosgene [i.e. C(S)Cl₂]. Similarly, the isocyanate functional group may be added to the radiometal chelator by reacting an amino group on the chelator with phosgene [i.e. C(O)Cl₂].

Non-peptide moieties (e.g. radiolabeling groups and/or albumin binders) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moeity may be clicked to the azide-containing peptide in the presence of Cu²⁺ and sodium ascorbate in water and organic solvent, such as acetonitrile (ACN) and DMF and the like. Non-peptide moieties may also be added in solution phase, which is routinely performed.

The synthesis of radiometal chelators is well-known and many chelators are commercially available (e.g. from Sigma-Aldrich™/Milipore Sigma™ and others). Protocols for conjugation of radiometals to the chelators is also well known (e.g. see Examples, below).

The synthesis of the BF₃-containing R¹¹ component of the compounds can be achieved following previously reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, 2019 60:1160-1166; each of which is incorporated by reference in its entirety). Generally, the BF₃-containing motif can be coupled to the linker via click chemistry by forming a 1,2,3-triazole ring between a BF₃-containing azido (or alkynyl) group and an alkynyl (or azido) group on the linker, or by forming an amide linkage between a BF₃-containing carboxylate and an amino group on the linker. To make the BF₃-containing azide, alkyne or carboxylate, a boronic acid ester-containing azide, alkyne or carboxylate is first prepared following by the conversion of the boronic acid ester to BF₃ in a mixture of HCl, DMF and KHF₂. For alkyl BF₃, the boronic acid ester-containing azide, alkyne or carboxylate can be prepared by coupling boronic acid ester-containing alkyl halide (such as iodomethylboronic acid pinacol ester) with an amine-containing azide, alkyne or carboxylate (such as N,N-dimethylpropargylamine). For aryl BF₃, the boronic acid ester can be prepared via Suzuki coupling using aryl halide (iodine or bromide) and bis(pinacolato) diboron.

¹⁸F-Fluorination of the BF₃-containing compounds via ¹⁸F-¹⁹F isotope exchange reaction can be achieved following previously published procedures (Liu et al. Nat Protoc 2015 10:1423-1432, incorporated by reference in its entirety). Generally, ˜100 nmol of the BF₃-containing compound is dissolved in a mixture of 15 μl of pyridazine-HCl buffer (pH=2.0-2.5, 1 M), 15 μl of DMF and 1 μl of a 7.5 mM KHF₂ aqueous solution. ¹⁸F-Fluoride solution (in saline, 60 μl) is added to the reaction mixture, and the resulting solution is heated at 80° C. for 20 min. At the end of the reaction, the desired product can be purified by solid phase extraction or by reversed high performance liquid chromatography (HPLC) using a mixture of water and acetonitrile as the mobile phase.

When the peptide has been fully synthesized on the solid support, the desired peptide may be cleaved from the solid support using suitable reagents, such as TFA, tri-isopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e. deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches.

To prepare the FAP-targeting ligands with R³═CN, an intermediate if can be prepared following the synthetic strategy shown in Scheme 1, below. CN-containing 1c can be obtained commercially or it can be prepared from the carboxylic acid 1a in 2 steps. The first step is converting the carboxylic acid 1a to amide 1b with (Boc)₂O and NH₄CO₃, and the second step is treating the amide 1b with trifluoroacetic anhydride to obtain cyanide 1c. Removing the Boc-protecting group in 1c such as treating with toluenesulfonic acid will obtain the free amine 1d. Coupling 1d with Boc-protected amino acid succinimide ester will yield 1e. Removing the Boc-protecting group in 1e such as treating with toluenesulfonic acid will obtain the CN-containing intermediate 1f.

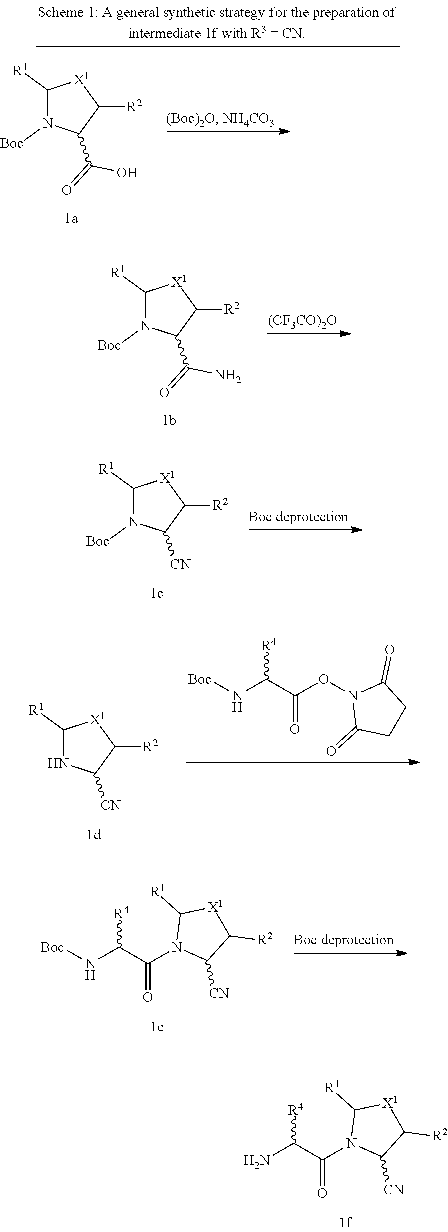

To prepare the FAP-targeting ligands with R³=boronic acid (—B(OH)₂), an intermediate 2e can be prepared following the synthetic strategy shown in Scheme 2, below. The Boc-protected boronic acid 2a is first treated with pinanediol to form 2b. Removing the Boc-protecting group in 2b such as treating with 4N HCl in dioxane will obtain the free amine 2c. Coupling 2c with Boc-protected amino acid succinimide ester will yield 2d. Removing the Boc-protecting group in 2d such as treating with toluenesulfonic acid will obtain the boronic acid-containing intermediate 2e.

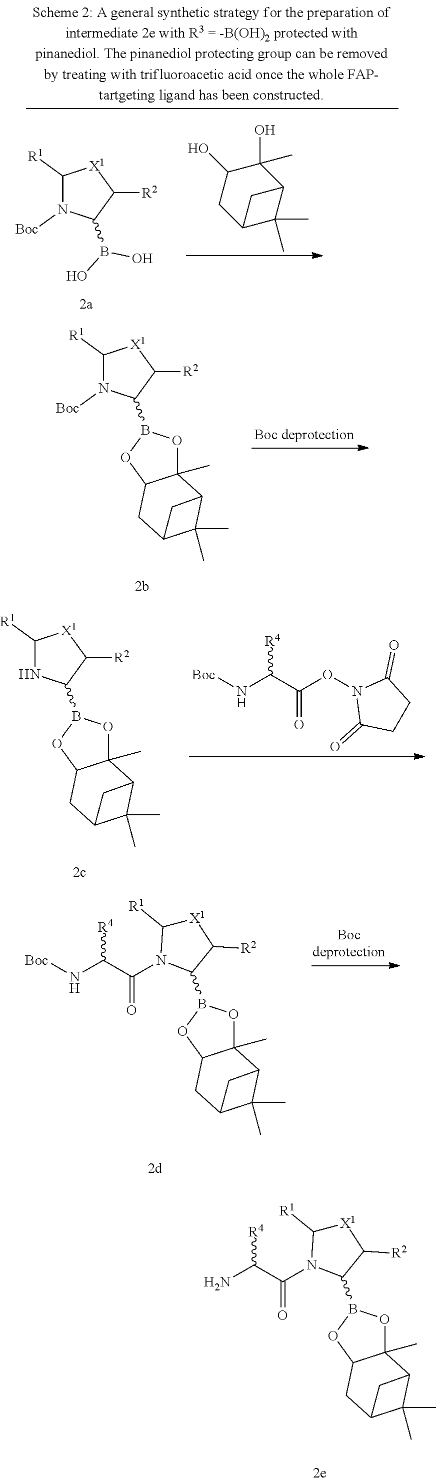

Forming an amide linkage (R⁶═—C(O)—) is shown in Scheme 3, below. A carboxylic group-containing tricyclic system 3a is first activated, for example by treating with N,N′-dicyclohexylcarbodiimide (DCC) and 2,3,5,6-tetrafluorophenol (TFP) to form the activated ester 3b. The activated ester 3b is then coupled with if (for R³═ON) or 2e (for R³═—B(OH)₂) to yield the FAP-targeting ligand 3c with an amide linkage (R⁶═—C(O)—).

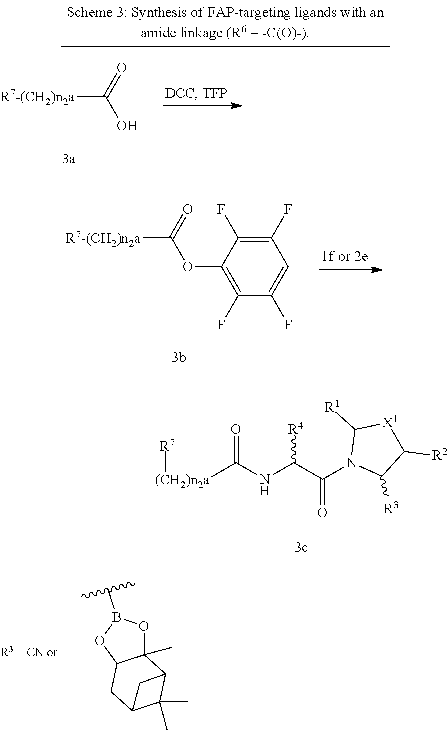

Forming a carbamate linkage (R⁶═—O—C(O)—) is shown in Scheme 4, below. The carboxylic group-containing tricyclic system 3a is first converted to a methyl ester 4b by treating with dry HCl in methanol. Treating the methyl ester 4b with LiAlH₄ will yield alcohol 4c. Treating alcohol 4c with 4-nitrophenyl chloroformate will yield 4d. Coupling 4d with if (for R³═ON) or 2e (for R³═—B(OH)₂) will yield the FAP-targeting ligand 4e with a carbamate linkage (R⁶═—O—C(O)—).

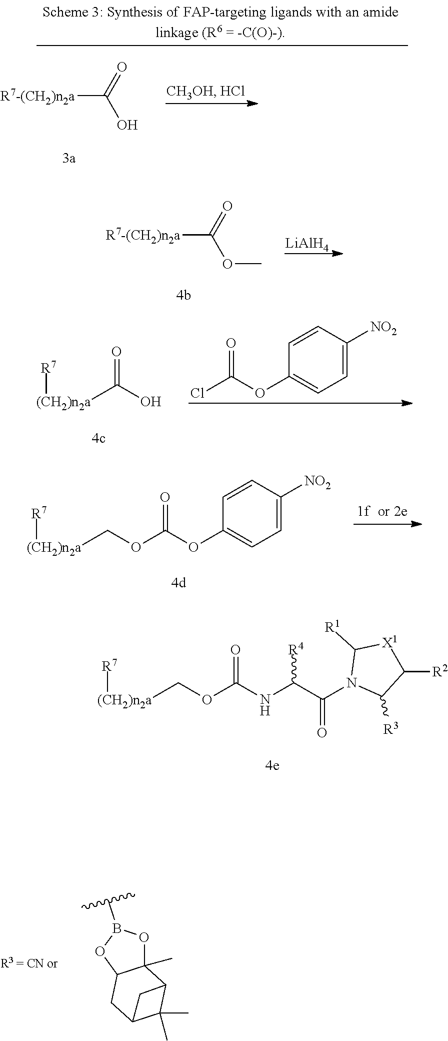

Forming a urea linkage (R⁶═—NH—C(O)—) is shown in Scheme 5, below. The alcohol-containing tricyclic system 4c is first converted to a mesylate 5a by treating with methanesulfonyl chloride (MsCl). Treating the mesylate 5a with excess ammonia will yield amine 5b. Treating amine 5b with 4-nitrophenyl chloroformate will yield 5c. Coupling 5c with if (for R³═CN) or 2e (for R³═—B(OH)₂) will yield the FAP-targeting ligand 5d with a urea linkage (R⁶═—NH—C(O)—).

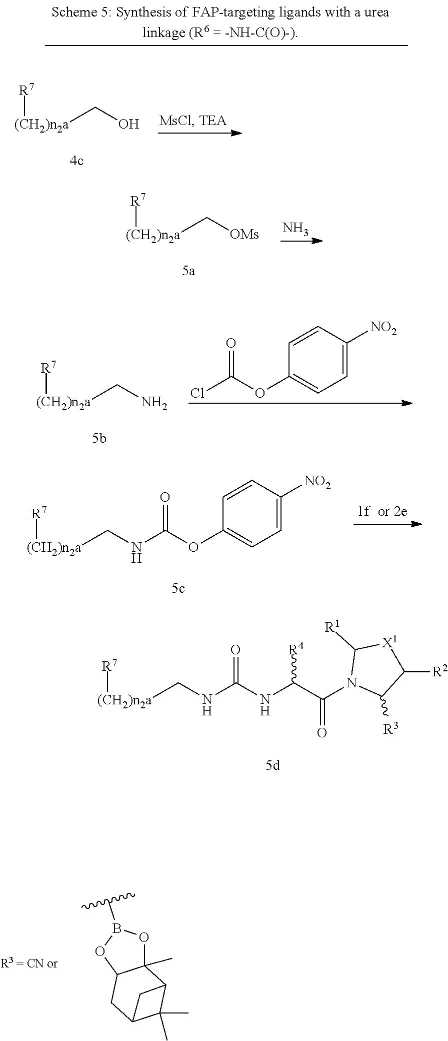

Scheme 6 gives examples for the preparation of carboxylic group-containing tricyclic systems such as compound 3a used in the above schemes. A substituted naphthylamine (6a or 7a) is first treated with methyl vinyl ketone, HCl, ZnCl₂ and FeCl₃ to form a tricyclic system. If a substituted 1-naphthylamine 6a is used, a benzo[h]quinoline derivative 6b is obtained. If a substituted 2-naphthylamine 7a is used, a mixture of benzo[g]quinoline derivative 7b and benzo[f]quinoline derivative 8b are obtained. Compounds 6b, 7b and 8b can be oxidized to form aldehyde 6c, 7c and 8c, respectively, by treating with SeO₂. The aldehyde 6c, 7c and 8c can be further oxidized to form carboxylic acid 6d, 7d and 8d, respectively, by treating with KMnO₄.

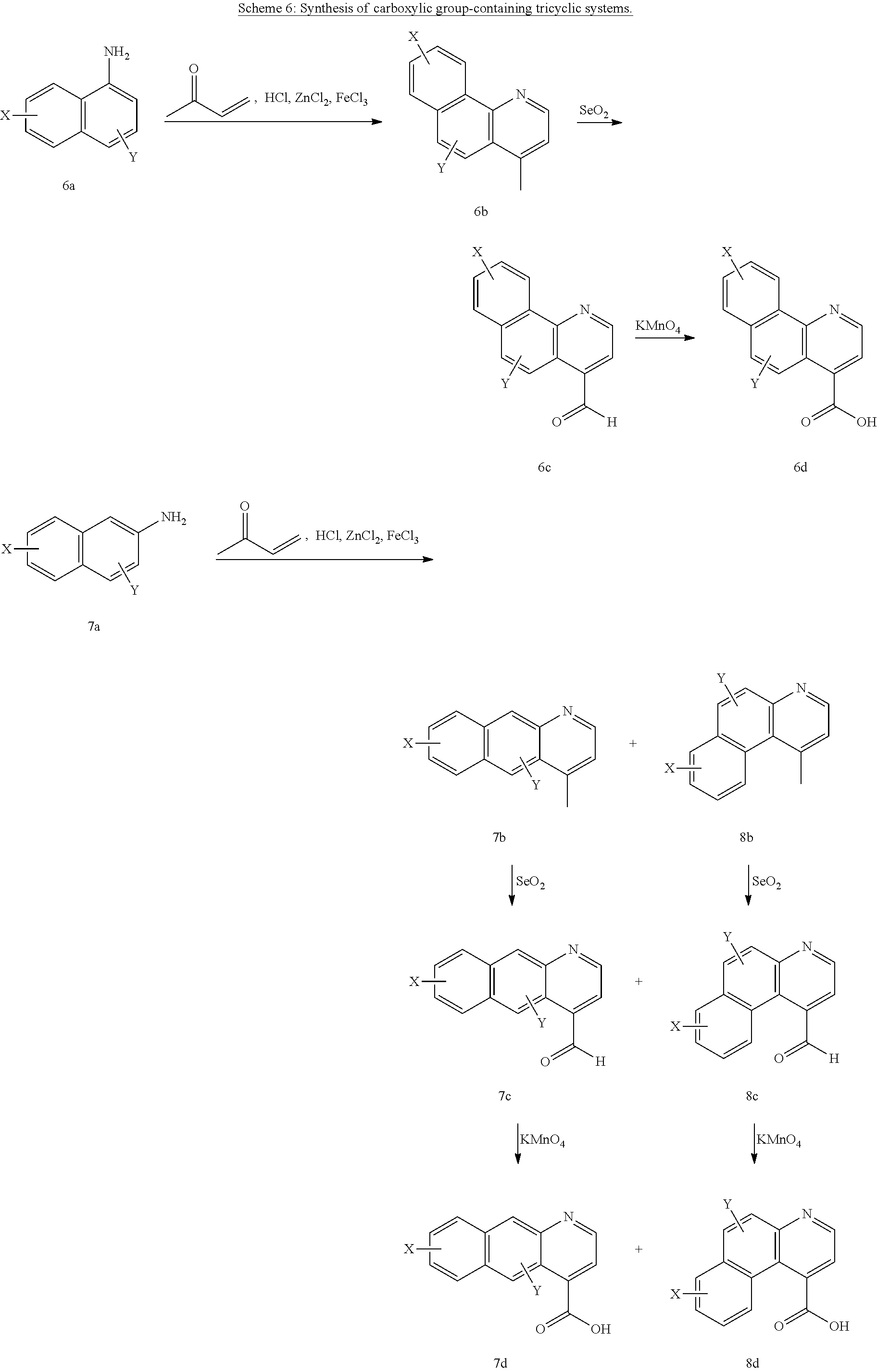

Coupling of the FAP-targeting ligands shown in Schemes 3-5 with a linker can be conducted in either solution or solid phase. The type of linkage formed between the linker and the tricyclic system depends on the substituted functional group (X or Y of compounds 6d, 7d and 8d, Scheme 6) on the tricyclic system. If the substituted functional group is an amino group, the formed linkage could be an amine, amide, urea or thiourea linkage. For the formation of amine, amide, urea or thiourea linkage, the amino group-containing tricyclic system would react with the alkyl halide (such as bromide or chloride)-, carboxylic group-, isocyanate- or thioisocyanate-containing linker, respectively. If the substituted functional group is a carboxylic group, the formed linkage could be an amide linkage. This could be accomplished by coupling the carboxylic group-containing tricyclic system with an amino group-containing linker. If the substituted functional group is a hydroxyl group, the formed linkage could be an ether linkage by coupling the hydroxyl group-containing tricyclic system with an alkyl halide (such as chloride or bromide)-containing linker. If the substituted functional group is a sulfhydryl group, the formed linkage could be a thioether or thiol-maleimide linkage. The thioether or thiol-maleimide linkage could be achieved by treating the sulfhydryl group-containing tricyclic system with an alkyl halide (such as chloride or bromide)- or maleimide-containing linker. If the substituted functional group is an alkyne or an azide, the formed linkage could be a triazole linkage via the copper-mediated click reaction between an alkyne- or an azide-containing tricyclic system with an azide- or an alkyne-containing tricyclic linker, respectively.

Synthetic schemes for preparing exemplary compounds SB03178 and SB04033 are shown below.

### General Methods

4-Methoxy-1-naphthalenamine (1) and (2S)-1-(2-aminoacetyl)-4,4-difluoro-2-pyrrolidinecarbonitrile 4-methylbenzenesulfonate were synthesized according to the literature procedures [26-27]. All other chemicals were procured from commercial sources and used without further purification. Purification and quality control of radiolabeling precursor, nonradioactive Ga-complexed standards and ⁶⁸Ga-labeled tracers were performed on Agilent (Santa Clara, CA) HPLC systems equipped with a model 1200 quaternary pump, a model 1200 UV absorbance detector (set at 220 nm), and a Bioscan (Washington, DC) Nal scintillation detector. The operation of Agilent HPLC systems was controlled using the Agilent ChemStation software. HPLC columns used were a semipreparative column (Luna C18, 5 μm particle size, 100 Å pore size, 250×10 mm) and an analytical column (Luna C18, 5 μm particle size, 100 Å pore size, 250×4.6 mm) from Phenomenex (Torrance, CA). The collected HPLC eluates containing the desired products were lyophilized using a Labconco (Kansas City, MO) FreeZone 4.5 Plus freeze drier. Mass analyses were performed using an AB SCIEX (Framingham, MA) 4000 QTRAP mass spectrometer system with an ESI ion source. C18 Sep-Pak cartridges (1 cm³, 50 mg) were obtained from Waters (Milford, MA). ⁶⁸Ga was eluted from an iThemba Laboratories (Somerset West, South Africa) generator and purified according to the previously published procedures using a DGA resin column from Eichrom Technologies LLC (Lisle, IL) [28]. Radioactivity of radiolabeled ligands was measured using a Capintec (Ramsey, NJ) CRC-25R/W dose calibrator. The radioactivity of mouse tissues collected from biodistribution studies was counted using a PerkinElmer (Waltham, MA) Wizard2 2480 automatic gamma counter.

### Synthesis of SB03178 and SB04033

The chemical structures of SB03178 and SB04033 are shown below.

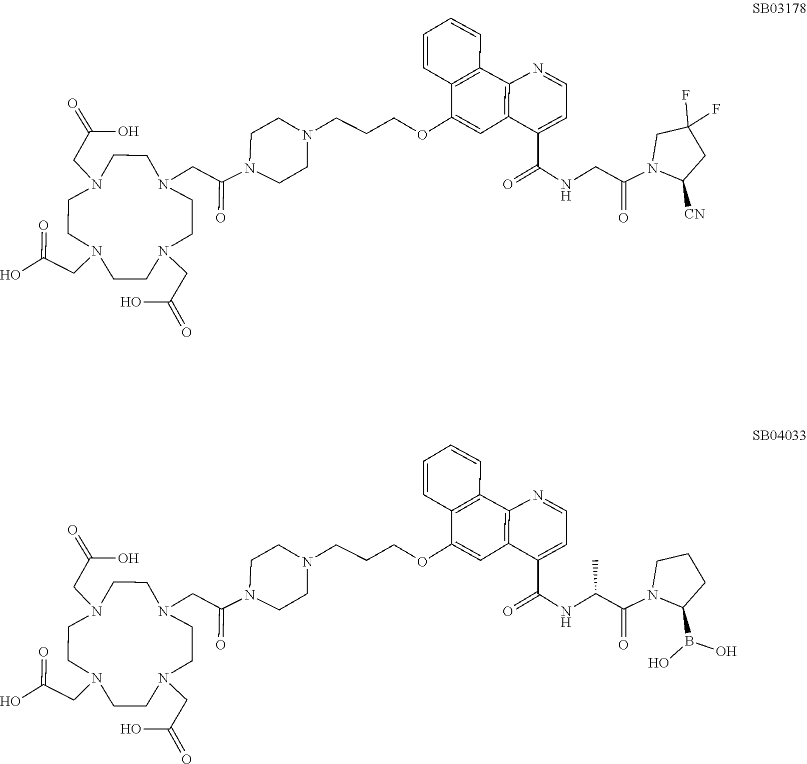 [SMILES: N#C[C@@H]1CC(F)(F)CN1C(=O)CNC(=O)c1ccnc2c1cc(OCCCN1CCN(C(=O)CN3CCN(CC(=O)O)CCN(CC(=O)O)CCN(CC(=O)O)CC3)CC1)c1ccccc12 ; C[C@@H](NC(=O)c1ccnc2c1cc(OCCCN1CCN(C(=O)CN3CCN(CC(=O)O)CCN(CC(=O)O)CCN(CC(=O)O)CC3)CC1)c1ccccc12)C(=O)N1CCC[C@H]1B(O)O]

Synthesis of SB03178. Synthesis of SB03178 was conducted using a multi-step synthetic approach as depicted in Scheme 7, shown below. 20 mg (31.41 umol) of compound 9 was Boc deprotected by treating it with 2 mL of 1:1 TFA/CH₂Cl₂ for 1 h at room temperature. The reaction mixture was evaporated and redissolved in 3 mL 2:1 H₂O/CH₃CN and neutralized by dropwise addition of triethylamine. DOTA-NHS (45 mg, 57 μmol) was added and the reaction was stirred overnight at room temperature. The crude was purified with HPLC (C18 semi-prep column, 4.5 mL/min, 20% CH₃CN (0.1% TFA), retention time: 12 min) and lyophilized to give a yellow powder. Yield: 11%. ESI-MS: calculated [M+H]⁺ for SB03178 C₄₄H₅₆F₂N₁₀O₁₀ 924.0; found 923.3.

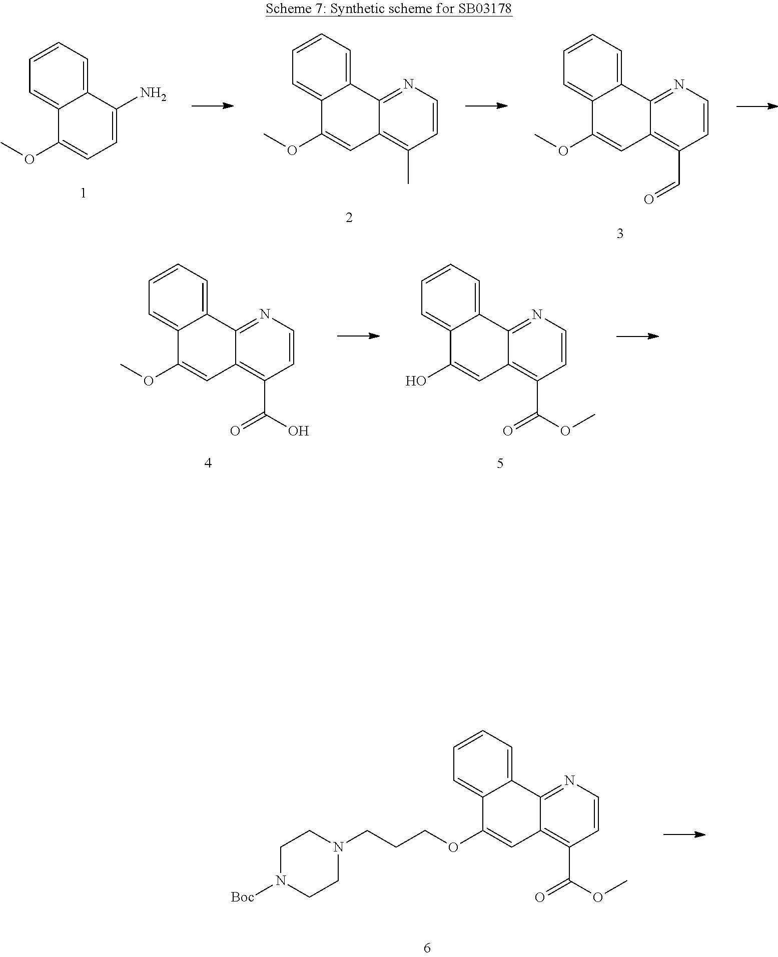 [SMILES: COc1ccc(N)c2ccccc12 ; COc1cc2c(C)ccnc2c2ccccc12 ; COc1cc2c(C=O)ccnc2c2ccccc12 ; COc1cc2c(C(=O)O)ccnc2c2ccccc12 ; COC(=O)c1ccnc2c1cc(O)c1ccccc12 ; COC(=O)c1ccnc2c1cc(OCCCN1CCN(C(=O)OC(C)(C)C)CC1)c1ccccc12]

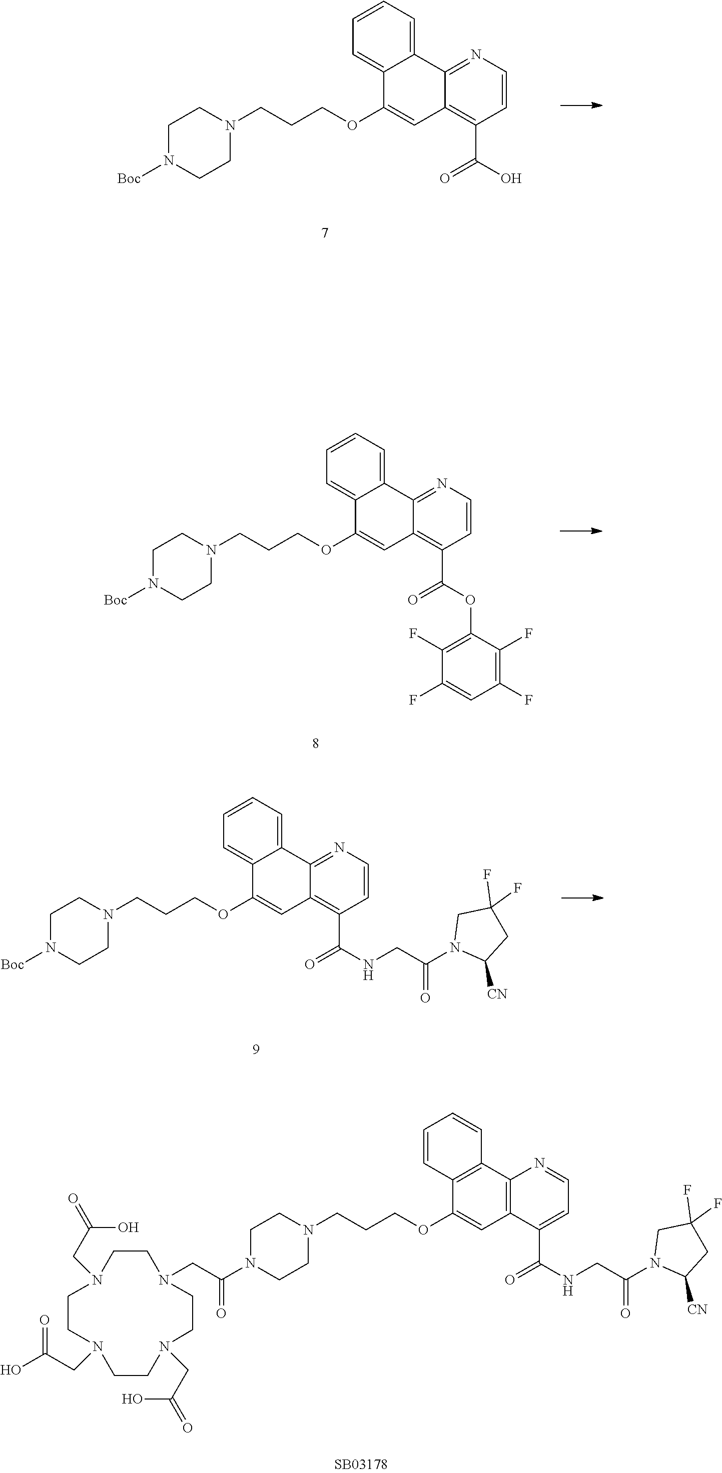 [SMILES: CC(C)(C)OC(=O)N1CCN(CCCOc2cc3c(C(=O)O)ccnc3c3ccccc23)CC1 ; CC(C)(C)OC(=O)N1CCN(CCCOc2cc3c(C(=O)Oc4c(F)c(F)cc(F)c4F)ccnc3c3ccccc23)CC1 ; CC(C)(C)OC(=O)N1CCN(CCCOc2cc3c(C(=O)NCC(=O)N4CC(F)(F)C[C@H]4C#N)ccnc3c3ccccc23)CC1 ; N#C[C@@H]1CC(F)(F)CN1C(=O)CNC(=O)c1ccnc2c1cc(OCCCN1CCN(C(=O)CN3CCN(CC(=O)O)CCN(CC(=O)O)CCN(CC(=O)O)CC3)CC1)c1ccccc12]

Synthesis of compound 2. 4-Methoxy-1-naphthalenamine 1 (1.87 g, 10.8 mmol) in ethanol (40 mL) was added 4N HCl in dioxane (3 mL) and the solution was stirred at room temperature for 15 min. ZnCl₂ (0.15 g, 1.1 mmol) and FeCl₃ (3.15 g, 19.4 mmol) were then added, and the resulting solution was stirred at 65° C. for 30 min. After addition of methyl vinyl ketone (841 mg, 12 mmol), the solution was heated at 80° C. for 24 h. The solution was cooled down, diluted with water (50 mL) and adjusted to pH 9 with 5N NaOH solution. After addition of CH₂Cl₂ (100 mL), the solution was stirred for 10 min, and then filtered through celite. The aquesous phase was extracted with CH₂Cl₂ (100 mL×2) and the CH₂Cl₂ fractions were combined, dried over MgSO₄ and purified by flash column chromatography using 1:9 ethyl acetate/hexanes to 15:85 ethyl acetate/hexanes to obtain 1.52 g (63% yield) of compound 2 as a white solid.

Synthesis of compound 3. Compound 2 (1.52 g, 6.8 mmol) and SeO₂ (755 mg, 6.8 mmol) in a mixture of water (10 mL) and 1,4-dioxane (40 mL) was heated at 105° C. for 46 h. Additional SeO₂ (1.51 g, 13.6 mmol) was added, and the solution was heated at 105° C. for 4 h. The solution was cooled down and extracted with ethyl acetate (100 mL). The ethyl acetate fraction was washed with saturated NaHCO₃ aqueous solution (100 mL), dried over MgSO₄ and evaluated to obtain 1.41 g (87% yield) of compound 3 as an orange solid.

Synthesis of compound 4. KMnO₄ (2.56 g, 16.2 mmol) in water (40 mL) was added dropwise to a solution of compound 3 (1.41 g, 5.9 mmol) in pyridine (40 mL) cooled in a ice/water bath. After completion of the addition, the resulting solution was stirred at room temperature for 24 h. The solution was filtered and the solid was wash with acetone (30 mL×3). The filtrate was evaporated and the residue was dissolved in water (100 mL). The solution was adjusted to pH 3 with concentrated HCl, and the resulting precipitate was collected to obtain 635 mg (42% yield) of compound 4 as a yellow solid.

Synthesis of compound 5. Compound 4 (600 mg, 2.4 mmol) in 48% HBr aqueous solution (50 mL) was heated at 110° C. for 3 days. After evaporation, the residue was dissolved in methanol (50 mL), and SOCl₂ (3 mL) was added dropwise. The solution solution was heated at 60° C. for 22 h and evaporated. Saturated NaHCO₃ aqueous solution (80 mL) was added to the residue, and the mixture was stirred for 10 min. The precipitate was collected by filtration, washed with water (10 mL×3) and dried to obtain 569 mg (94% yield) of compound 5 as a yellow solid.

Synthesis of compound 6. A mixture of compound 5 (569 mg, 2.2 mmol), triphenylphosphine (656 mg, 2.5 mmol) and 1-Boc-4-(3-hydroxypropyl)piperazine (611 mg, 2.5 mmol) in tetrahydrofuran (20 mL) was added dropwise a solution of diisopropyl azodicarboxylate (506 mg, 2.5 mmol) in tetrahydrofuran (5 mL). The resulting solution was stirred at room temperature for 2 days, and purified by flash column chromatography to obtain 726 mg (67% yield) of compound 6 as a light brown solid.

Synthesis of compound 7. Compound 6 (726 mg, 1.5 mmol) and NaOH (1.07 g, 26.7 mmol) in a mixture of water (10 mL), methanol (10 mL) and tetrahydrofuran (10 mL) was stirred at room temperature for 23 h. After evaporation, the residue was dissolved in water (20 mL) and the resulting solution was adjusted to pH 5.5 with 1N HCl. The formed precipitate was collected by filtration and washed with water (5 mL×3) to obtain 625 mg (89% yield) compound 7 as a light brown solid.

Synthesis of compound 8. Dicyclohexylcarbodiimide (145 mg, 0.70 mmol) was added to a solution of compound 7 (310 mg, 0.67 mmol) and 2,3,5,6-tetrafluorophenol (133 mg, 0.8 mmol) in N,N-dimethylformamide. The resulting solution was stirred at room temperature for 3 days and filtered. The filtrate was evaporated and purified by flash column chromatography eluted with 2:8 ethyl acetate/hexanes to 3:7 ethyl acetate/hexanes to obtain 207 mg (50% yield) of compound 8 as a yellow solid.

Synthesis of compound 9. A mixture of compound 8 (207 mg, 0.34 mmol), (2S)-1-(2-aminoacetyl)-4,4-difluoro-2-pyrrolidinecarbonitrile 4-methylbenzenesulfonate (181 mg, 0.5 mmol), triethylamine (101 mg, 1.0 mmol), CH₂Cl₂ (4 mL) and acetonitrile (4 mL) was heated at 50° C. for 23 h. After evaporation, the residue was purified by flash column chromatography using 1:1 ethyl acetate/hexanes to 1:5 methanol/ethyl acetate to obtain 200 mg (92% yield) of compound 9 as a light brown solid.

Synthesis of Ga-SB03178. SB03178 (2 mg) was dissolved in 0.2 mL NaOAc buffer (0.1 N, pH 4.5) and GaCl₃ (5 eq., 42 μL, 0.265 M) was added. The reaction mixture was incubated at 90° C. for 30 min and then purified with HPLC (C18 semi-prep column, 4.5 mL/min, 21% CH₃CN (0.1% TFA), retention time: 10 min) and lyophilized to give a yellow powder. Yield: 32%. ESI-MS: calculated [M+H]²⁺ for Ga-SB03178 C₄₄H₅₄F₂GaN₁₀O₁₀ 495.8; found 495.2.

Synthesis of ⁶⁸Ga-SB03178. To a 4 mL reaction vial preloaded with 10 nmol of SB03178 in 0.65 mL HEPES buffer (2M, pH 5.0) was added purified ⁶⁸GaCl₃ (141.71 MBq) in 0.55 mL water. The reaction mixture was incubated in microwave oven for 1 min at power level 2. After cooling down for 1 min at ambient temperature, the mixture was then purified by HPLC equipped with the C18 semi-prep column, eluted with 20% acetonitrile (containing 0.1% TFA) and 80% deionized water (containing 0.1% TFA) at a flow rate of 4.5 mL/min and the retention time of ⁶⁸Ga-SB03178 is 20.1 min. The eluate fractions containing ⁶⁸Ga-SB03178 were collected, diluted with PBS (50 mL) and passed through a C18 Sep-Pak cartridge. ⁶⁸Ga-SB03178 trapped on the cartridge was eluted off with ethanol (containing 100 ppm ascorbic acid) and formulated with PBS (containing 100 ppm ascorbic acid) for animal studies. QC was performed on HPLC equipped with the C18 analytical column (2 mL/min, 23% acetonitrile (containing 0.1% TFA) and 77% deionized water (containing 0.1% TFA), retention time of ⁶⁸Ga-SB03178: 8.0 min). Decay-corrected radiochemical yield: 75%. Purity: >99%.

Synthesis of SB04033. Synthesis of SB04033 was conducted following a multi-step synthetic approach as depicted in Scheme 8, below. The pinanediol protecting group of 12 was removed by treating with cleavage cocktail containing 95% TFA, 2.5% H₂O/2.5% TIS for 4 h at room temperature. The crude was diluted with ether, evaporated and purified with HPLC (C18 semi-prep column, 4.5 mL/min, 17% CH₃CN (0.1% TFA) in H₂O, retention time: 11.8 min) and lyophilized to give SB04033 as a yellow solid. Yield: 74%.

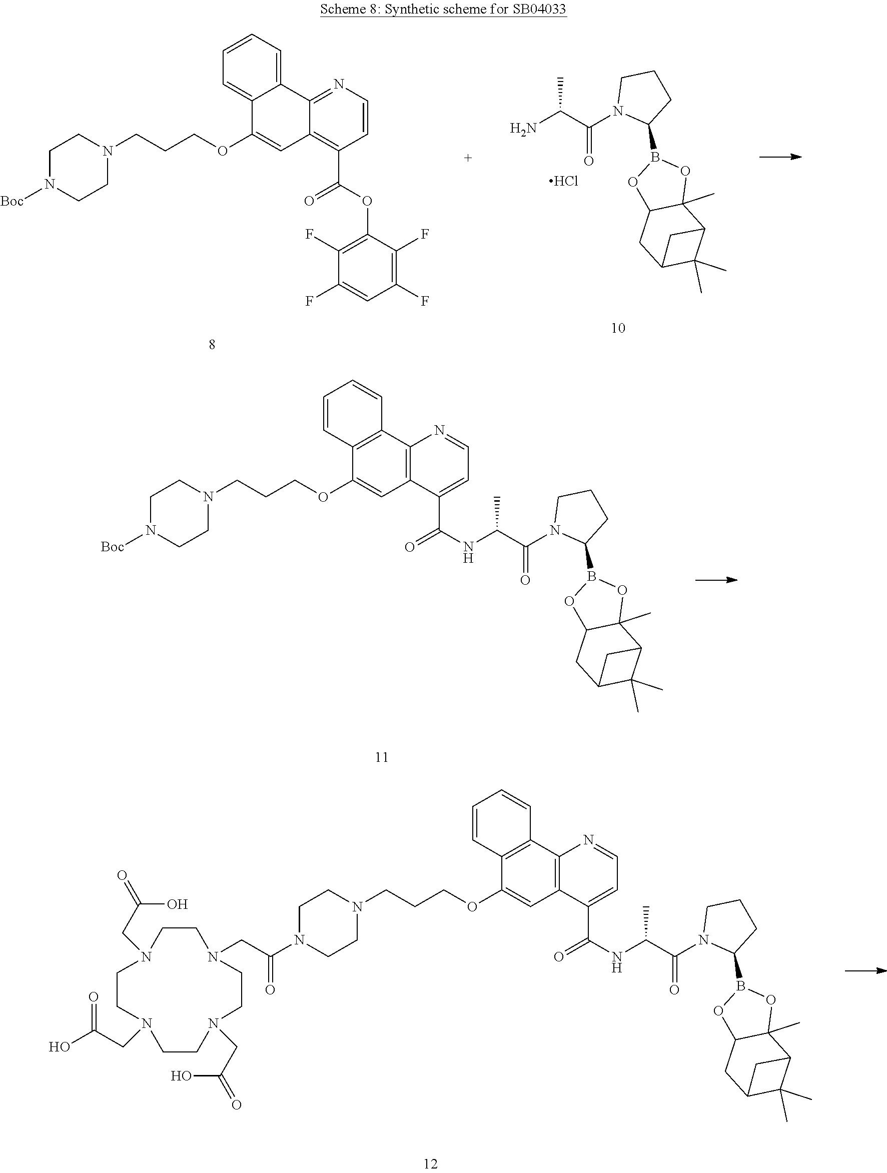 [SMILES: CC(C)(C)OC(=O)N1CCN(CCCOc2cc3c(C(=O)Oc4c(F)c(F)cc(F)c4F)ccnc3c3ccccc23)CC1 ; C[C@@H](N)C(=O)N1CCC[C@H]1B1OC2CC3CC(C3(C)C)C2(C)O1 ; Cl ; C[C@@H](NC(=O)c1ccnc2c1cc(OCCCN1CCN(C(=O)OC(C)(C)C)CC1)c1ccccc12)C(=O)N1CCC[C@H]1B1OC2CC3CC(C3(C)C)C2(C)O1 ; C[C@@H](NC(=O)c1ccnc2c1cc(OCCCN1CCN(C(=O)CN3CCN(CC(=O)O)CCN(CC(=O)O)CCN(CC(=O)O)CC3)CC1)c1ccccc12)C(=O)N1CCC[C@H]1B1OC2CC3CC(C3(C)C)C2(C)O1]

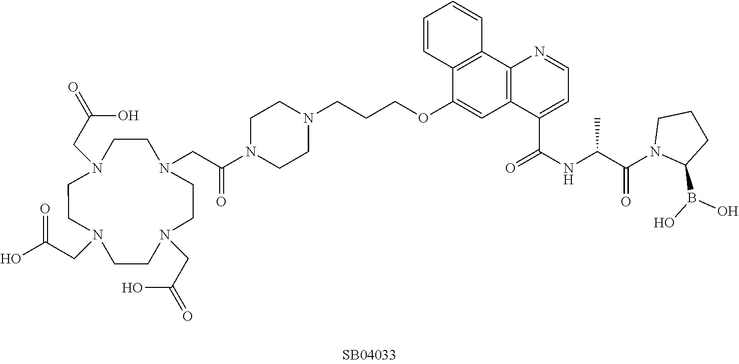 [SMILES: C[C@@H](NC(=O)c1ccnc2c1cc(OCCCN1CCN(C(=O)CN3CCN(CC(=O)O)CCN(CC(=O)O)CCN(CC(=O)O)CC3)CC1)c1ccccc12)C(=O)N1CCC[C@H]1B(O)O]

Synthesis of compound 11. A mixture of compound 10 (101 mg, 0.28 mmol) [29] and triethylamine (86 mg, 0.85 mmol, 118 ul) were dissolved in CH₃CN (5 mL). Compound 8 (133 mg, 0.22 mmol) was dissolved in CH₃CN (10 mL) and added to it. The reaction was stirred at 80° C. for 2 days. After evaporation, the residue was purified by flash column chromatography using 0-5% MeOH/ethyl acetate to obtain 111 mg (67% yield) of compound 11 as a yellow solid.

Synthesis of compound 12. Compound 11 was Boc deprotected using 4N HCl/dioxane:ether (1:1, 20 mL) by stirring overnight at room temperature. The reaction was evaporated and 41 mg (53 umol) was redissolved in 3-4 mL H₂O. DOTA-NHS (61 mg, 80 umol) was added and the reaction was stirred overnight at 55° C. after adjusting its pH to 8 by dropwise addition of triethylamine (˜60 uL). The crude was purified with HPLC (C18 prep column, 30 mL/min, gradient 0-80% CH₃CN (0.1% formic acid) in H₂O, retention time: 6.6 min) and lyophilized to give 12 as a white solid. Yield: 11%. ESI-MS: calculated [M+H]⁺ for C₅₄H₇₆BN₉O₁₂ 1054.06; found 1055.30.

Synthesis of Ga-SB04033. SB04033 (2 mg, 2.2 umol) was dissolved in 0.2 mL NaOAc buffer (0.1 N, pH 4.5) and GaCl₃ (5 eq., 41 μL, 0.27 M) was added. The reaction mixture was incubated at 90° C. for 30 min and then purified with HPLC (C18 semi-prep column, 4.5 mL/min, 17% CH₃CN (0.1% TFA) in H₂O, retention time: 13.1 min) and lyophilized to give a yellow solid. Yield: 77%.

Synthesis of ⁶⁸Ga-SB04033. To a 4 mL reaction vial preloaded with 10 nmol of SB04033 in 0.55 mL HEPES buffer (2M, pH 5.0) was added purified ⁶⁸GaCl₃ (189 MBq) in 0.55 mL water. The reaction mixture was incubated in microwave oven for 1 min at power level 2. After cooling down for 1 min at ambient temperature, the mixture was then purified by HPLC equipped with the C18 semi-prep column, eluted with 17% acetonitrile (containing 0.1% TFA) and 83% deionized water (containing 0.1% TFA) at a flow rate of 4.5 mL/min and the retention time of ⁶⁸Ga-SB04033 is 30.2 min. The eluate fractions containing ⁶⁸Ga-SB04033 were collected, diluted with PBS (50 mL) and passed through a C18 Sep-Pak cartridge. ⁶⁸Ga-SB04033 trapped on the cartridge was eluted off with ethanol (containing 100 ppm ascorbic acid) and formulated with PBS (containing 100 ppm ascorbic acid) for animal studies. QC was performed on HPLC equipped with the C18 analytical column (2 mL/min, 21% acetonitrile (containing 0.1% TFA) and 79% deionized water (containing 0.1% TFA), retention time of ⁶⁸Ga-SB04033: 7.22 min). Decay-corrected radiochemical yield: 28%. Purity: >95%.

### PET Imaging and Biodistribution Studies in Tumor-Bearing Mice

All imaging and biodistribution studies were performed using male NOD-scidIL2Rg^{null} (NRG) mice and conducted according to the guidelines established by the Canadian Council on Animal Care and approved by Animal Ethics Committee of the University of British Columbia. For tumor inoculations, mice were anesthetized by inhalation with 2% isoflurane in oxygen and implanted subcutaneously with 7.5×10⁶ HEK293T:hFAP or U87 cells below the left shoulder. Imaging and biodistribution studies were performed only after tumors grew to 5-8 mm in diameter.

For PET/CT imaging study, ˜6 MBq of the ⁶⁸Ga-labeled tracer was injected through the tail vein. At 45 min post-injection, imaging mouse was sedated again and positioned on the scanner. A 10-min CT scan was conducted for localization and attenuation correction for reconstruction. For 1-h and 3-h PET images, 10 min and 15 min acquisitions were performed on the mouse, respectively.

For ex vivo biodistribution studies, mice were injected with -1-3 MBq of the ⁶⁸Ga-labeled tracer. Mice were allowed to recover and roam freely in the cages after injecting the tracer. At 1 h post-injection, mice were euthanized, blood was drawn from heart, and organs/tissues of interest were collected, rinsed with PBS, blotted dry, weighed, and counted using an automated gamma counter. The uptake in each organ/tissue was normalized to the injected dose and expressed as the percentage of the injected dose per gram of tissue (% ID/g).

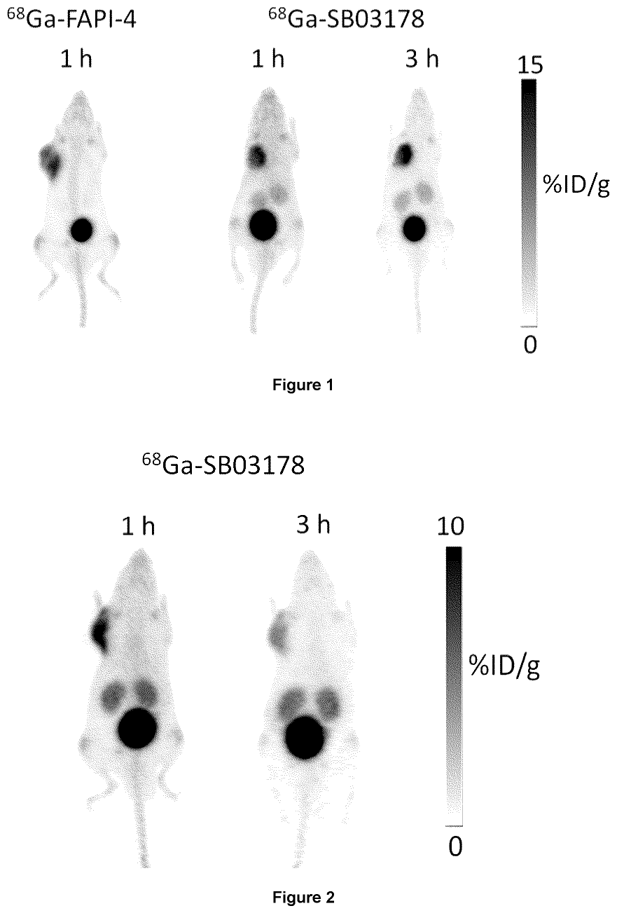

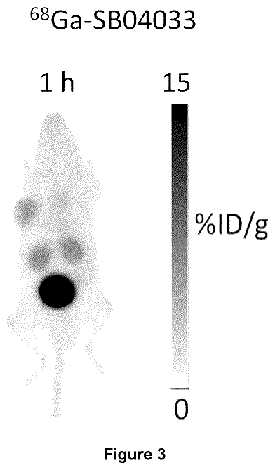

FIG. 1 shows representative maximum-intensity-projection PET images of ⁶⁸Ga-FAPI-4 and ⁶⁸Ga-SB03178 acquired at 1 and 3 h post-injection from HEK293T:hFAP tumor-bearing mice. FIG. 2 shows representative maximum-intensity-projection PET images of ⁶⁸Ga-SB03178 acquired at 1 and 3 h post-injection from U87 tumor-bearing mice. FIG. 3 shows a representative maximum-intensity-projection PET image of ⁶⁸Ga-SB04033 acquired at 1 post-injection from HEK293T:hFAP tumor-bearing mice.

Biodistribution data for ⁶⁸Ga-SB03178, ⁶⁸Ga-SB04033, and ⁶⁸Ga-FAPI-4 in mice bearing FAP tumor xenografts are presented in Tables 5-7. Data are presented as mean±SD % ID/g.

TABLE 5
Biodistribution (at 1 h post-injection) of ⁶⁸Ga-SB03178 and
⁶⁸Ga-FAPI-4 in mice bearing HEK293T:hFAP tumor xenografts.

| | ⁶⁸Ga-SB03178 | ⁶⁸Ga-FAPI-4 |
|---|---|---|
| Blood | 3.00 ± 0.38 | 1.09 ± 0.08 |
| Fat | 0.25 ± 0.04 | 0.11 ± 0.02 |
| Testes | 0.51 ± 0.09 | 0.29 ± 0.04 |
| Spleen | 1.00 ± 0.35 | 0.57 ± 0.13 |
| Pancreas | 0.66 ± 0.06 | 0.38 ± 0.04 |
| Stomach | 0.32 ± 0.22 | 0.07 ± 0.02 |
| Liver | 1.29 ± 0.17 | 0.37 ± 0.02 |
| Adrenal glands | 3.48 ± 3.31 | 1.09 ± 0.31 |
| kidneys | 11.1 ± 1.40 | 1.86 ± 0.10 |
| Heart | 0.82 ± 0.10 | 0.35 ± 0.03 |
| Lungs | 1.92 ± 0.26 | 0.75 ± 0.09 |
| Tumor | 24.3 ± 4.44 | 12.7 ± 2.19 |
| Bone | 2.18 ± 0.35 | 3.39 ± 1.03 |
| Muscle | 0.65 ± 0.09 | 0.68 ± 0.04 |
| Brain | 0.07 ± 0.01 | 0.05 ± 0.01 |

TABLE 6
Biodistribution (at 1 and 3 h post-injection) of ⁶⁸Ga-SB03178
in mice bearing HEK293T:hFAP tumor and U87 xenografts.

| | HEK293T:hFAP | | U87 | |
|---|---|---|---|---|
| | tumor model | | tumor model | |
| | 1 h (n = 4) | 3 h (n = 2) | 1 h (n = 3) | 3 h (n = 3) |
| Blood | 2.59 ± 0.32 | 1.35 ± 1.18 | 3.55 ± 0.22 | 0.98 ± 0.09 |
| Fat | 0.22 ± 0.03 | 0.11 ± 0.10 | 0.28 ± 0.18 | 0.09 ± 0.04 |
| Testes | 0.44 ± 0.08 | 0.12 ± 0.03 | 0.65 ± 0.09 | 0.25 ± 0.03 |
| Small | 1.04 ± 0.14 | 0.34 ± 0.21 | 1.07 ± 0.03 | 0.56 ± 0.16 |
| Intestine | | | | |
| Large | 0.52 ± 0.21 | 0.53 ± 0.13 | 0.44 ± 0.09 | 1.15 ± 0.37 |
| Intestines | | | | |
| Stomach | 0.27 ± 0.19 | 0.06 ± 0.02 | 0.28 ± 0.12 | 0.24 ± 0.02 |
| Pancreas | 0.57 ± 0.05 | 0.17 ± 0.05 | 0.65 ± 0.11 | 0.27 ± 0.06 |
| Spleen | 0.87 ± 0.30 | 0.24 ± 0.07 | 0.48 ± 0.08 | 0.34 ± 0.04 |
| Adrenal Glands | 3.01 ± 2.86 | 0.31 ± 0.06 | 1.41 ± 0.38 | 0.49 ± 0.30 |
| Kidneys | 9.58 ± 1.21 | 3.89 ± 1.35 | 8.92 ± 1.32 | 9.63 ± 1.23 |
| Liver | 1.11 ± 0.15 | 0.33 ± 0.13 | 1.12 ± 0.08 | 0.71 ± 0.05 |
| Heart | 0.71 ± 0.09 | 0.15 ± 0.06 | 0.80 ± 0.06 | 0.28 ± 0.01 |
| Lungs | 1.66 ± 0.23 | 0.39 ± 0.08 | 1.66 ± 0.10 | 0.72 ± 0.11 |
| Tumor | 21.0 ± 3.84 | 6.58 ± 1.98 | 16.3 ± 1.72 | 7.95 ± 0.29 |
| Muscle | 0.56 ± 0.08 | 0.13 ± 0.08 | 0.57 ± 0.21 | 0.34 ± 0.07 |
| Bone | 1.89 ± 0.31 | 0.38 ± 0.07 | 2.51 ± 0.66 | 1.11 ± 0.27 |
| Brain | 0.06 ± 0.01 | 0.02 ± 0.00 | 0.07 ± 0.01 | 0.03 ± 0.00 |

TABLE 7
Biodistribution (at 1 post-injection) of ⁶⁸Ga-SB04033
in mice bearing HEK293T:hFAP tumor xenografts.

| | ⁶⁸Ga-SB04033 |
|---|---|
| | 1 h (n = 4) |
| Blood | 3.62 ± 0.47 |
| Fat | 0.30 ± 0.15 |
| Testes | 0.59 ± 0.07 |
| Small Intestine | 1.53 ± 0.20 |
| Large Intestines | 0.33 ± 0.03 |
| Stomach | 0.30 ± 0.10 |
| Pancreas | 0.62 ± 0.05 |
| Spleen | 0.98 ± 0.27 |
| Adrenal Glands | 0.65 ± 0.46 |
| Kidneys | 10.3 ± 0.55 |
| Liver | 1.06 ± 0.11 |
| Heart | 0.95 ± 0.12 |
| Lungs | 2.13 ± 0.33 |
| HEK293T:hFAP Tumor | 5.95 ± 0.16 |
| Muscle | 0.54 ± 0.11 |
| Bone | 1.52 ± 0.22 |
| Brain | 0.08 ± 0.01 |

Conclusions. ⁶⁸Ga-SB03178 outperformed ⁶⁸Ga-FAPI-4 (current gold standard) in biodistribution and PET-imaging studies, demonstrating higher uptake in HEK293T:hFAP tumor xenografts (e.g. see Table 5: 24.3±4.44% ID/g for ⁶⁸Ga-SB03178 vs 12.7±2.19% ID/g for ⁶⁸Ga-FAPI-4 at 1 h post-injection). Similar results are seen for ⁶⁸Ga-SB03178 in U87 xenografts (e.g. see Table 6). Modifying the pyrrolidine group by removal of both fluorines and/or replacement of cyano with boronic acid leads to weaker uptake (e.g. see Table 7 data for ⁶⁸Ga-SB04033), but the tricyclic ring structure nevertheless represents an improvement for all FAP-targeting tracers that have monocyclic or bicyclic fused ring structures at position R⁷.

### NUMBERED REFERENCES

1) Šedo, A.; Malik, R. Dipeptidyl peptidase IV-like molecules: homologous proteins or homologous activities? Biochimca et Biophysica Acta—Protein Structure and Molecular Enzymology 2001; 1550: 107-116.
2) Bušek P, Malik R, Šedo A. Dipeptidyl peptidase IV activity and/or structure homologues (DASH) and their substrates in cancer. International Journal of Biochemistry & Cell Biology 2004; 36: 408-421.
3) Scanlan M J, Raj B K M, Calvo B, Garin-Chesa P, Sanz-Moncasi M P, Healey J H, Old L J, Rettig W J. Molecular cloning of fibroblast activation protein α, a member of the serine protease family selectively expressed in stromal fibroblasts of epithelial cancers. Proceedings of the National Academy of Science of the United States of America 1994; 91: 5657-5661.
4) Cohen J, Chesa P G, Beresford H R, Feickert H J, Jennings M T, Oettgen H F, Old L J. Differential expression of cell surface antigens and glial fibrillary acidic protein in human astrocytoma subsets. Cancer Research 1986; 46: 6406-6412.
5) Rettig W J, Garin-Chesa P, Healey J H, Su S L, Ozer H L, Schwab M, Albino A P, Old L J. Regulation and heteromeric structure of the fibroblast activation protein in normal and transformed cells of mesenchymal and neuroectodermal origin. Cancer Reserach 1993; 53: 3327-3335.
6) Rettig W J, Garin-Chesa P, Beresford H R, Oettgen H F, Melamed M R, Old L J. Cell-surface glycoproteins of human sarcomas: Differential expression in normal and malignant tissues and cultured cells. Proceedings of the National Academy of Science of the United States of America 1988; 85: 3110-3114.
7) Huang Y, Wang S, Kelly T. Seprase promotes rapid tumor growth and increased microvessel density in a mouse model of human breast cancer. Cancer Research 2004; 64: 2712-2716.
8) Kraman M, Bambrough P J, Arnold J N, Roberts E W, Magiera L, Jones J O, Gopinathan A, Tuveson D A, Fearon D T. Suppression of antitumor immunity by stromal cells expressing fibroblast activation protein-α. Science 2010; 330: 827-830.
9) Monsky W L, Lin C Y, Aoyama A, Kelly T, Akiyama S K, Mueller S C, Chen W T. A potential marker protease of invasiveness, seprase, is localized on invadopodia of human malignant melanoma cells. Cancer Res 1994; 54: 5702-5710.
10) Kelly T, Mueller S C, Yeh Y, Chen W T. Invadopodia promote proteolysis of a wide variety of extracellular matrix proteins. Journal of Cellular Physiology 1994; 158: 299-308.
11) Kawase T, Yasui Y, Nishina S, Hara Y, Yanatori I, Tomiyama Y, Nakashima Y, Yoshida K, Kishi F, Nakamura M, Hino K. Fibroblast activation protein-α-expressing fibroblasts promote the progression of pancreatic ductal adenocarcinoma. BMC Gastroenterology 2015; 15: 109.
12) Shi M, Yu D-H, Chen Y, Zhao C-Y, Zhang J, Liu Q-H, Ni C-R, Zhu M-H. Expression of fibroblast activation protein in human pancreatic adenocarcinoma and its clinicopathological significance. World Journal of Gastroenterology 2012; 18: 840-846.
13) Lee H-O, Mullins S R, Franco-Barraza J, Valianou M, Cukierman E, Cheng J D. FAP-overexpressing fibroblasts produce an extracellular matrix that enhances invasive velocity and directionality of pancreatic cancer cells. BMC Cancer 2011; 11: 245.
14) Liu R, Li H, Liu L, Yu J, Ren X. Fibroblast activation protein. Cancer Biology & Therapy 2012; 13: 123-129.
15) Pure E. The road to integrative cancer therapies: emergence of a tumor-associated fibroblast protease as a potential therapeutic target in cancer. Expert Opinion on Therapeutic Targets 2009; 13: 967-973.
16) Santos A M, Jung J, Aziz N, Kissil J L, Puré E. Targeting fibroblast activation protein inhibits tumor stromagenesis and growth in mice. Journal of Clinical Investigation 2009; 119: 3613-3625.
17) Brennen W N, Rosen D M, Chaux A, Netto G J, Isaacs J T, Denmeade S R. Pharmacokinetics and toxicology of a fibroblast activation protein (FAP)-activated prodrug in murine xenograft models of human cancer. The Prostate 2014; 74: 1308-1319.
18) LeBeau A M, Brennen W N, Aggarwal S, Denmeade S R. Targeting the cancer stroma with a fibroblast activation protein-activated promelittin protoxin. Molecular Cancer Therapeutics 2009; 8; 1378-1386.
19) Welt S, Divgi C R, Scott A M, Garin-Chesa P, Finn R D, Graham M, Carswell E A, Cohen A, Larson S M, Old L J, Rettig W J. Antibody targeting in metastatic colon cancer: a phase I study of monoclonal antibody F19 against a cell-surface protein of reactive tumor stromal fibroblasts. Journal of Clinical Oncology 1994; 12: 1193-1203.
20) Fischer E, Chaitanya K, Wuest T, Wadle A, Scott A M, van den Broek M, Schibli R, Bauer S, Renner C. Clinical Cancer Research 2012; 18: 6208-6218.
21) Giesel F L, Kratochwil C, Lindner T, Marschalek M M, Loktev A, Lehnert W, Debus J, Jäger D, Flechsig P, Altmann A, Mier W, Haberkorn U. FAPI-PET/C T: biodistribution and preliminary dosimetry estimate of two DOTA-containing FAP-targeting agents in patients with various cancers. Journal of Nuclear Medicine 2019; 60: 386-392.
22) Lindner T, Loktev A, Altmann A, Giesel F, Kratochwil C, Debus J, Jäger D, Mier W, Haberkorn U. Development of quinoline based theranostic ligands for the targeting of fibroblast activation protein. Journal of Nuclear Medicine 2018; 59: 1415-1422.
23) Loktev A, Lindner T, Mier W, Debus J, Altmann A, Jäger D, Giesel F, Kratochwil C, Barthe P, Roumestand C, Haberkorn U. A new method for tumor imaging by targeting cancer associated fibroblasts. Journal of Nuclear Medicine 2018; 59: 1423-1429.
24) Kratochwil C, Flechsig P, Lindner T, Abderrahim L, Altmann A, Mier W, Adeberg S, Rathke H, Röhrich M, Winter H, Plinkert P K, Marme F, Lang M, Kauczor H U, Jager D, Debus J, Haberkorn U, Giesel F L. FAPI-PET/C T: Mean intensity of tracer-uptake (SUV) in 28 different kinds of cancer. Journal of Nuclear Medicine 2019; 60: 801-805.
25) Loktev A, Lindner T, Burger E M, Altmann A, Giesel F, Kratochwil C, Debus J, Marmé F, Jäger D, Mier W, Haberkorn U. Development of novel FAP-targeted radiotracers with improved tumor retention. Journal of Nuclear Medicine 2019; 60: 1421-1429.
26) Knoelker H-J, Bauermeister M, Pannek J-B, Wolpert M. Transition metal-diene complexes in organic synthesis. Part 22. The iron-mediated quinine imine cyclization: a general route to 3-hydroxyca rbazoles. Synthesis 1995; 4: 397-408.
27) Jasen K, Heirbaut L, Verkerk R, Cheng J D, Joossens J, Cos P, Maes L, Lambeir A-M, De Meester I, Augustyns K, Van der Veken P. Extended structure-activity relationship and pharmacokinetic investigation of (4-quinolinoyl)glycyl-2-cyanopyrrolidine inhibitors of fibroblast activation protein (FAP). Journal of Medicinal Chemistry 2014; 57: 3053-3074.
28) Lin K S, Pan J, Amouroux G, Turashvili G, Mesak F, Hundal-Jabal N, Pourghiasian M, Lau J, Jenni S, Aparicio S, Benard F. In vivo radioimaging of bradykinin receptor B1, a widely overexpressed molecule in human cancer. Cancer Research 2015; 75: 387-393.
29) Bachovchin W W, Lai H S, Wu W. FAP-targeted radiopharmaceuticals and imaging agents, and uses related thereto. WO 2021/195198 A1.

The present invention has been described with regard to one or more embodiments. However, it will be apparent to persons skilled in the art that a number of variations and modifications can be made without departing from the scope of the invention as defined in the following claims. The scope of the invention should therefore not be limited by the preferred embodiments set forth in the above Examples, but should be given the broadest interpretation consistent with the description as a whole.

All documents cited in this document are incorporated by reference in their entirety. All priority documents are incorporated by reference in its entirety.

1. A compound comprising n1 radiolabeling groups and n2 fibroblast activation protein (FAP)-targeting groups, wherein the radiolabeling groups and the FAP-targeting groups are connected through n3 linkers;
n1 and n2 are each independently 1-6;
n3 is 1-11;
each of the FAP-targeting groups is represented by R^{FAP} and independently has the structure of Formula (I) or a pharmaceutically acceptable salt of Formula (I): 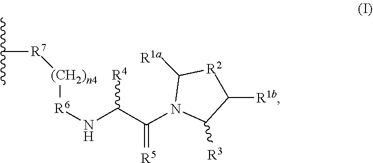
wherein:
R^{1a} is —H, —OH, halogen, C₁₋₆ alkyl, —O—C₁₋₆ alkyl, or —S—C₁₋₆ alkyl;
R^{1b} is —H, —OH, halogen, C₁₋₆ alkyl, —O—C₁₋₆ alkyl, or —S—C₁₋₆ alkyl;
R² is —NH—, —N(CH₃)—, —CH₂—, —CH(OH)—, —CHF—, —CF₂—, —S—, or —O—;
R³ is —CO₂H, —C(O)NH₂, —CN or —B(OH)₂;
R⁴ is —H, methyl, or ethyl;
R⁵ is ═O;
R⁶ is —C(O)—, —O—C(O)—, or —NH—C(O)—;
n4 is 0, 1, 2, or 3; and
R⁷ is an 11 to 15-membered aromatic, partially aromatic, or non-aromatic fused tricyclic system, wherein the tricyclic system is optionally contains 1-6 heteroatoms selected from N, O, and/or S, and is optionally substituted with 1-5 —OH, —NO₂, —CN, —NH₂, —NH(CH₃), —N(CH₃)₂, halogen, C₁₋₆ alkyl, —O—C₁₋₆ alkyl, or —S—C₁₋₆ alkyl;
each of the radiolabeling groups is represented by R^{rad}, wherein each R^{rad} is independently: a radiometal chelator; an aryl or heteroaryl substituted with a radiohalogen; a prosthetic group containing a trihaloborate; a prosthetic group containing a silicon-halogen-acceptor moiety; or a prosthetic group containing a halophosphate, halosulfate, or sulfonyl halide;
each of the linkers is represented by R⁸, optionally wherein each R⁸ is a linear or branched chain of n5 units of R^{L1}, each unit of R^{L1} separated from each other by a unit of L¹, and each unit of R^{L1} optionally connected to an additional unit of L¹ to form a branching point, wherein each FAP-targeting group is connected to R⁸ through a unit of L³, and each R^{rad} is connected to R⁸ through a unit of L¹ or incorporates L¹ or a portion of L¹, wherein:
n5 is 1-20;
each R^{L1} is, independently, a linear, branched, and/or cyclic Cₙ₆ alkylenyl, alkenylenyl and/or alkynylenyl, wherein each n6 is independently 1-20, wherein any carbon bonded to two other carbons is optionally independently replaced by N, S, or O, and carbons are optionally independently substituted with oxo, hydroxyl, sulfhydryl, —SeH, halogen, guanidino, amine, amide, urea, carboxylic acid, sulfonic acid, sulfinic acid, or phosphoric acid;
L¹ bonds to carbon, wherein each L¹ is independently —O—, —S—, —N(R^{L2})—, —N(R^{L2})C(O)—, —N(R^{L2})C(S)—, —C(O)N(R^{L2})—, —C(S)N(R^{L2})—, —NH—C(O)—NH—, —NH—C(S)—NH—, 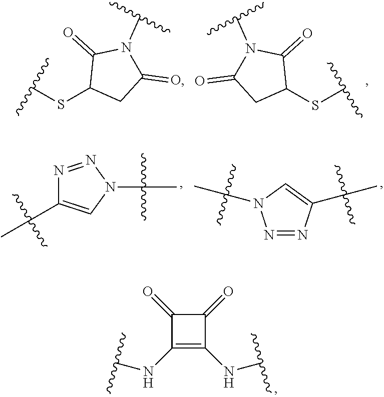
or L²;
each R^{L2} is independently H, methyl, or ethyl;
each L² is an N-containing heterocycle independently selected from the group consisting of: 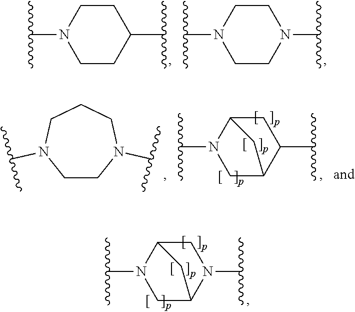
wherein each p is independently 1 or 2; and
an albumin binder (R^{alb}) is optionally bonded to an L¹ of the linker, wherein the albumin binder is:
(CH₂)ₙ₇—CH₃ wherein n7 is 8-20;
(CH₂)ₙ₈—C(O)OH wherein n8 is 8-20; 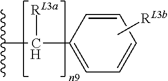
wherein n9 is 1-4 and R^{L3a} is H or methyl, and R^{L3b} is I, Br, F, Cl, H, OH, OCH₃, NH₂, NO₂ or C₁-C₆ alkyl; or 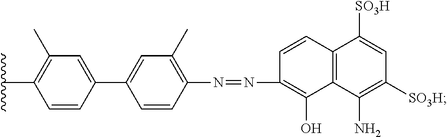 [SMILES: *c1ccc(-c2ccc(N=Nc3ccc4c(S(=O)(=O)O)cc(S(=O)(=O)O)c(N)c4c3O)c(C)c2)cc1C]
each L³ is independently:
absent, —O—, —S—, —N(R^{L2})—, —N(R^{L2})C(O)—, —N(R^{L2})C(S)—, —C(O)N(R^{L2})—, —C(S)N(R^{L2})—, —NH—C(O)—NH—, —NH—C(S)—NH—, 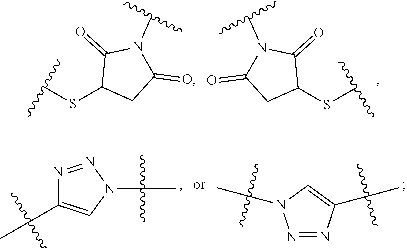
or L³ is absent, —C(O)—, —C(S)—, —N(R^{L2})C(O)—, —N(R^{L2})C(S)—, if bonded to nitrogen of the tricyclic system.

2. The compound of claim 1, wherein:
n1 is 2 and each radiolabeling group is the same or different;
n2 is 2 and each FAP-targeting group is the same or different; and
n3 is 1.

3. The compound of claim 1, wherein the compound has the structure of Formula (II) or is a pharmaceutically acceptable salt of Formula (II): 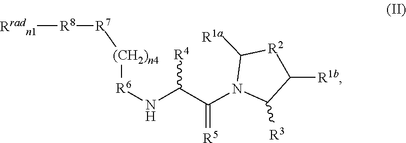
wherein n1, n4, R^{1a}, R^{1b}, R², R³, R⁴, R⁵, R⁶, R⁷, R⁸, and R^{rad} are as defined in claim 1.

4. The compound of claim 1, wherein each FAP-targeting group has the structure of Formula (Ia) or a pharmaceutically acceptable salt of Formula (Ia): 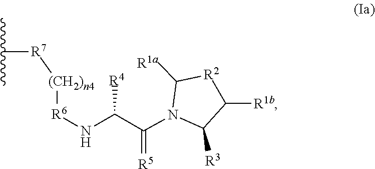
wherein n4, R^{1a}, R^{1b}, R², R³, R⁴, R⁵, R⁶, and R⁷ are as defined in claim 1.

5. The compound of claim 1, wherein at least one n4 is 0.

6. The compound of claim 1, wherein at least one R^{1a} and R^{1b} are both hydrogen.

7. The compound of claim 1, wherein at least one R² is CH₂, CHF, CF₂, or S.

8. The compound of claim 1, wherein at least one R³ is —CN.

9. The compound of claim 1, wherein at least one R⁴ is —H or methyl.

10. The compound of claim 1, wherein at least one R⁶ is —C(O)—.

11. The compound of claim 1, wherein:
at least one R⁷ is: 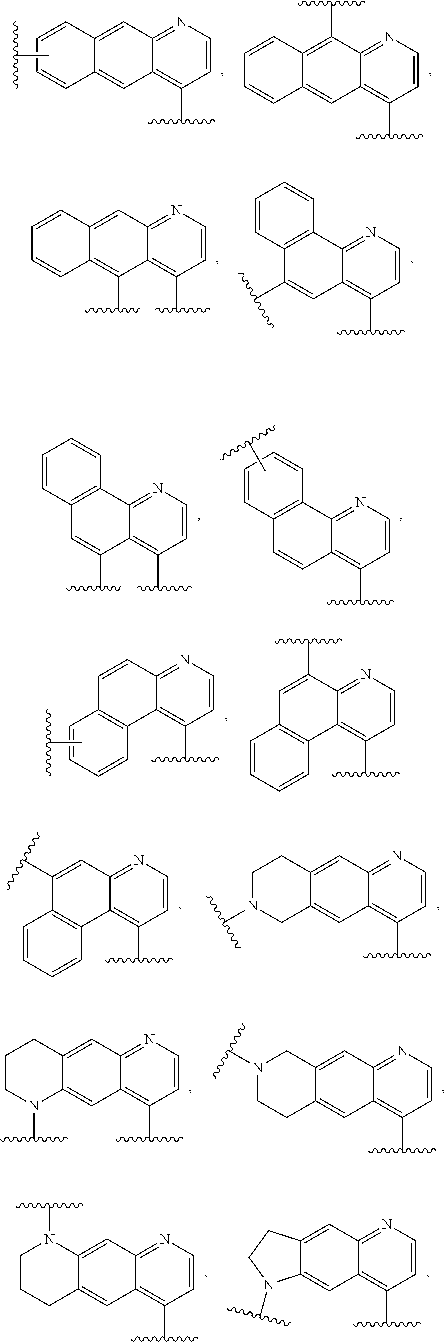 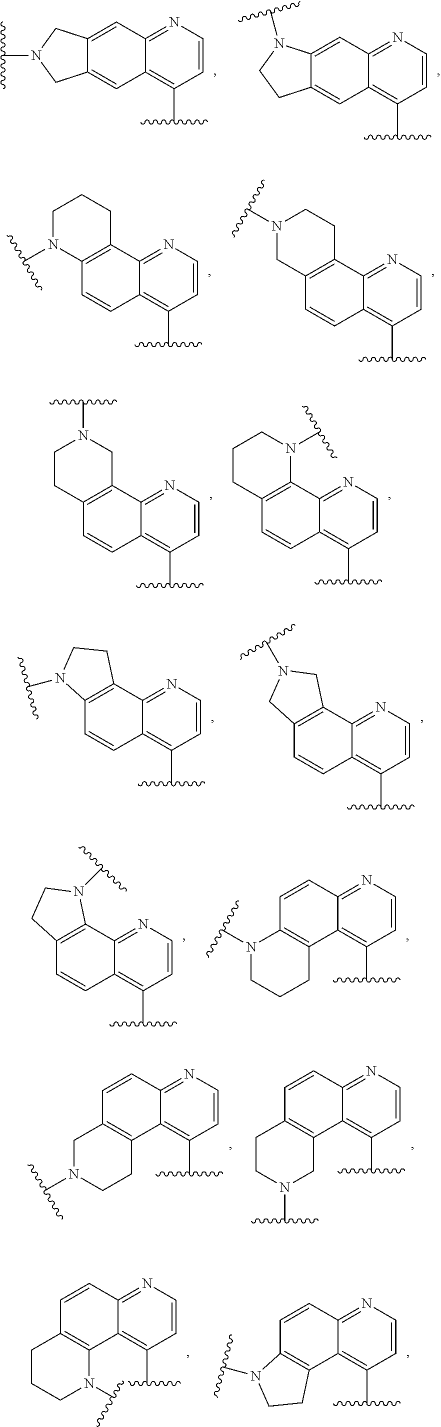 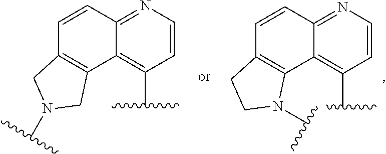 [SMILES: *c1ccnc2ccc3c(c12)CN(*)C3 ; *c1ccnc2ccc3c(c12)N(*)CC3]
wherein the tricyclic system optionally contains 1-3 additional heteroatoms selected from N, O, and/or S, and is optionally substituted with 1-5 —OH, —NO₂, —CN, —NH₂, —NH(CH₃), NH(CH₃)₂, halogen, C₁₋₆ alkyl, —O—C₁₋₆ alkyl, or —S—C₁₋₆ alkyl; or
at least one R⁷ is:

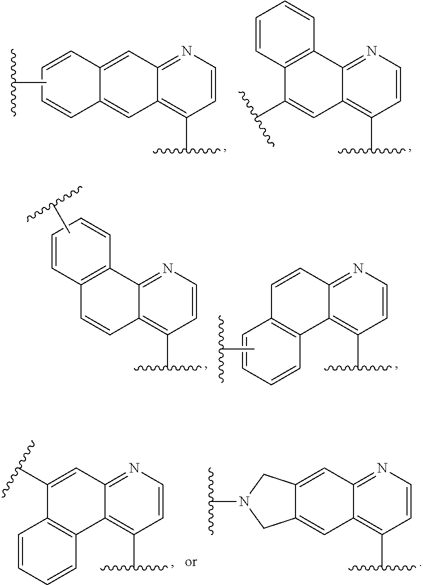

12. The compound of claim 1, at least one R⁸ is -L^{2′}-R^{L1′}-L^{3′}- wherein:
L^{2′} is 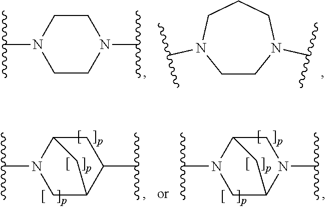
wherein each p is independently 1 or 2, optionally wherein L^{2′} is; 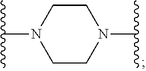 [SMILES: *N1CCN(*)CC1]
R^{L1′} is C₂₋₁₀ alkylenyl; and
L^{3′} is —O—.

13. The compound of claim 1, wherein:
(a) R^{rad}ₙ₁—R⁸— is configured as shown in Formula III: 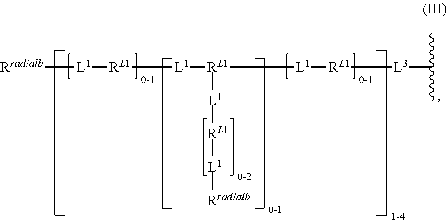
wherein L¹, R^{L1}, and L³ are as defined in claim 1, and R^{rad/alb} is either R^{rad} or R^{alb}, and
wherein 0-1 R^{rad/alb} is R^{alb}; or
(b) the compound has the structure of Formula IV, or is a pharmaceutically acceptable salt thereof: 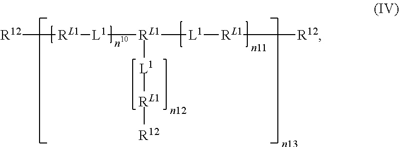
wherein:
n10, n11, and n12 are each independently 0-3;
n13 is 1-4;
each R¹² is independently -L¹-R^{rad}, -L¹-R^{alb}, or -L³-R^{FAP}, wherein at least one R¹² is -L¹-R^{rad} and at least one R¹² is -L³-R^{FAP}, optionally wherein at least one R¹² is -L¹-R^{alb}; and
R^{rad}, R^{alb}, R^{FAP}, L¹, R^{L1}, and L³ are as defined in claim 1; or
(c) the compound has the structure of Formula V, or is a pharmaceutically acceptable salt thereof: 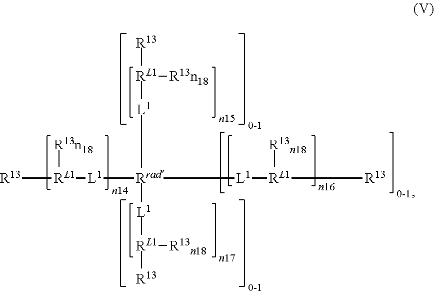
wherein:
R^{rad′} is a radiometal chelator that optionally contains multiple functional groups for attachment to a linker or linkage group (e.g. DOTA, and the like);
each R¹³ is independently -L¹-R^{rad}, -L¹-R^{alb}, or -L³-R^{FAP}, wherein at least one R¹³ is -L³-R^{FAP}, optionally wherein at least one R¹³ is -L¹-R^{alb}; and
n14, n15, n16, and n17 are each independently is 0-3;
each n18 is 0 or 1;
R^{rad}, R^{alb}, R^{FAP}, L¹, R^{L1}, and L³ are as defined in claim 1.

14. The compound of claim 1, wherein at least one R⁸ forms a linear or branched peptide linker: (Xaa¹⁰)₁₋₂₀, wherein each Xaa¹⁰ is independently a proteinogenic or non-proteinogenic amino acid residue, wherein each peptide backbone amino or amide group is independently optionally methylated, and wherein each non-proteinogenic amino acid residue is independently selected from Table 1.

15. The compound of claim 1, wherein at least one R^{rad} is a radiometal chelator, optionally selected from the group consisting of: DOTA and derivatives; DOTAGA; NOTA; NOTAGA; EDTA; Neunpa; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof, H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; H₆phospa; a trithiol chelate; mercaptoacetyl; hydrazinonicotinamide (HYNIC); dimercaptosuccinic acid; 1,2-ethylenediylbis -L-cysteine diethyl ester; methylenediphosphonate; hexamethylpropyleneamineoxime; hexakis(methoxy isobutyl isonitrile), H4py4pa-phenyl-NCS, and Crown.

16. The compound of claim 15, wherein the radiometal chelator is bound by a radiometal, a radionuclide-bound metal, or a radionuclide-bound metal-containing prosthetic group, optionally selected from the group consisting of: ⁶⁸Ga, ⁶¹Cu, ⁶⁴Cu, ⁶⁷Cu, ⁶⁷Ga, ^{110m}In, ¹¹¹In, ⁴⁴Sc, ⁸⁶Y, ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ⁷²As, ⁷⁷As, ²¹¹At, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{114m}In, ^{94m}Tc, ^{99m}Tc, ¹⁴⁹Tb, ¹⁵²Tb, ¹⁵⁵Tb, ¹⁶¹Tb, ¹⁵³Sm, ²²³Ra, ²²⁴Ra, and [¹⁸F]AlF.

17. The compound of claim 1, wherein at least one R^{rad} is a trifluoroborate containing prosthetic group R¹¹—R¹⁰—, wherein R¹⁰ is —(CH₂)₁₋₅— and optionally methylene, and wherein R¹¹ is: 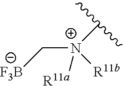
wherein R^{11a} and R^{11b} are each independently a C₁-C₅ linear or branched alkyl group, 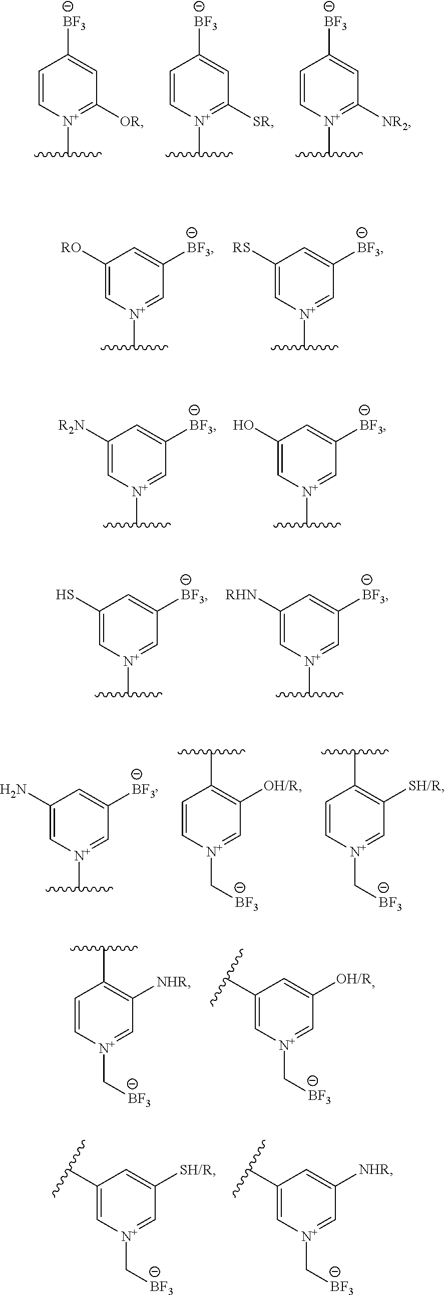 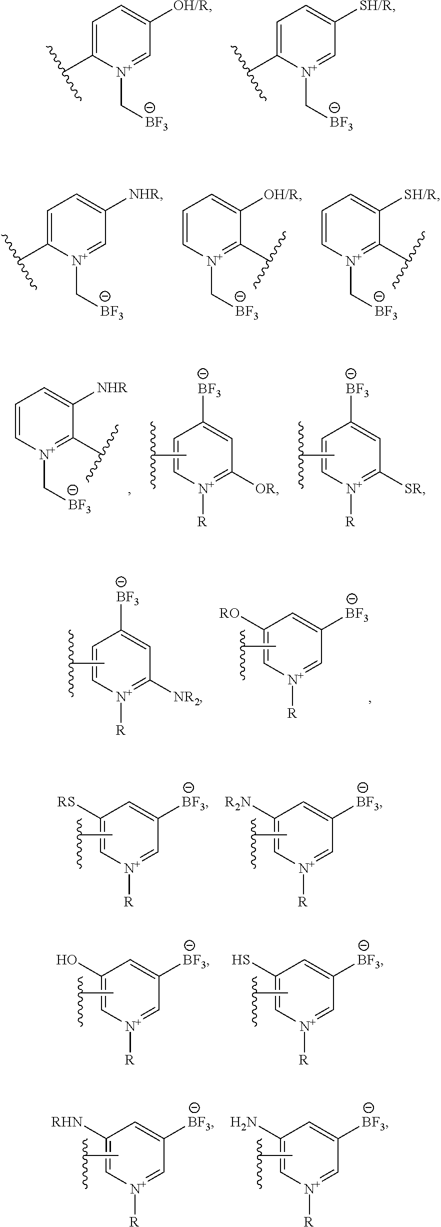 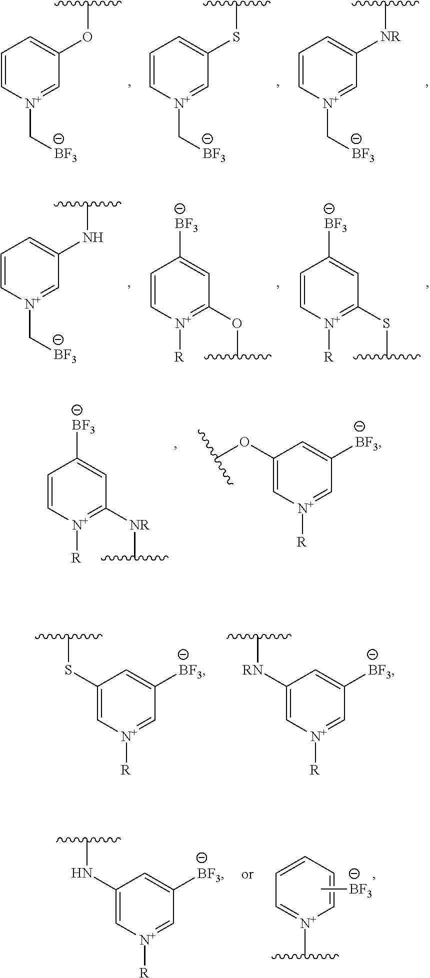
in which the R in each pyridine substituted —OR, —SR, —NR—, —NHR or —NR₂ is independently a branched or linear C₁-C₅ alkyl, optionally wherein the fluorines in R¹¹ comprise ¹⁸F.

18. The compound of claim 1, which is SB03178 or SB04033: 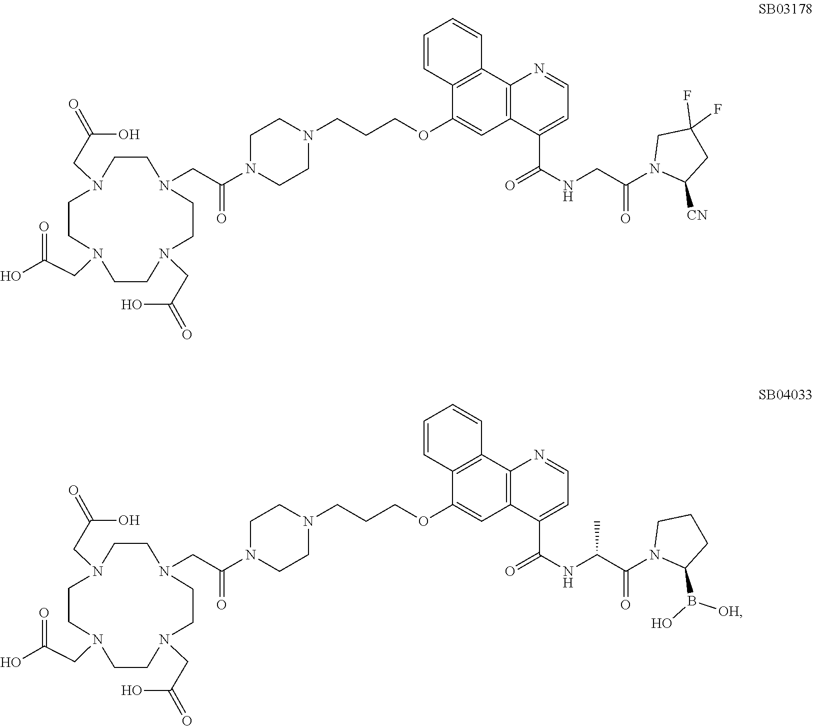 [SMILES: N#C[C@@H]1CC(F)(F)CN1C(=O)CNC(=O)c1ccnc2c1cc(OCCCN1CCN(C(=O)CN3CCN(CC(=O)O)CCN(CC(=O)O)CCN(CC(=O)O)CC3)CC1)c1ccccc12 ; C[C@@H](NC(=O)c1ccnc2c1cc(OCCCN1CCN(C(=O)CN3CCN(CC(=O)O)CCN(CC(=O)O)CCN(CC(=O)O)CC3)CC1)c1ccccc12)C(=O)N1CCC[C@H]1B(O)O]
or a pharmaceutically acceptable salt thereof.

19. The compound of claim 1, for use in treatment of a FAP-expressing tumor in a subject, wherein at least one R^{rad} is bound by a therapeutic radiometal, optionally wherein the therapeutic radiometal is ¹⁶⁵Er, ²¹²Bi, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹²Bi, ²¹³Bi, ²¹²Pb, ⁴⁷SC, ⁹⁰Y, ²²⁵Ac, ^{117m}Sn, ¹⁵³Sm, ¹⁴⁹Tb, ¹⁶¹Tb, ²²⁴Ra, ²²⁷Th, ²²³Ra, ¹⁸⁸Re, ¹⁸⁶Re, ²¹¹At, ¹³¹I, ⁶⁴Cu, or ⁶⁷Cu.

20. The compound of claim 1, for use in imaging FAP-expressing tissues in a subject, wherein at least one R^{rad} comprising a positron or gamma emitting radionuclide, optionally wherein the positron or gamma emitting radionuclide is ⁶⁸Ga, ⁶⁷Ga, ⁶¹Cu, ⁶⁴Cu, ^{94m}Tc, ^{99m}Tc, ¹⁰⁵Rh, ^{110m}In, ¹¹¹In, ⁴⁴Sc, ⁸⁶Y, ⁸⁹Zr, ⁹⁰Nb, ¹⁵²Tb, ¹⁵⁵Tb, ²⁰³Pb, ¹⁸F, ¹³¹I, ¹²³I, ¹²⁴I or ⁷²As.
